(19) United States
(12) Patent Application Publication
(10) Pub. No.: US 2009/0069300 A1
(43) Pub. Date: Mar. 12, 2009

(54) ISOQUINOLINYL AND ISOINDOLINYL DERIVATIVES AS HISTAMINE-3 ANTAGONISTS

(75) Inventors: Dahui Zhou, East Brunswick, NJ (US); Jonathan Laird Gross, Cranbury, NJ (US); Albert Jean Robichaud, Ringoes, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/208,794

(22) Filed: Sep. 11, 2008

Related U.S. Application Data
(60) Provisional application No. 60/993,636, filed on Sep. 12, 2007.

(51) Int. Cl. A61K 31/55; C07D 401/06; A61K 31/4725; C07D 401/14; C07D 403/06; A61K 31/4164

(52) U.S. Cl. 514/217.07; 546/141; 514/309; 514/308; 546/140; 548/305.1; 514/394; 540/597

(57) ABSTRACT

The present invention provides a compound of formula I and the use thereof for the treatment of a central nervous system disorder related to or affected by the histamine-3 receptor.

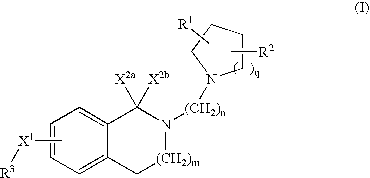

### CROSS-REFERENCE TO RELATED APPLICATIONS

This application claims the benefit under 35 U.S.C. §119(e) to co-pending U.S. provisional application No. 60/993,636, filed Sep. 12, 2007, which is hereby incorporated by reference in its entirety.

### FIELD OF THE INVENTION

The current invention relates to isoquinolinyl and isoindolinyl compounds, their use in modulation of the histamine-3 (H₃) receptor and treatment of a variety of central nervous system disorders related to or affected by the H₃ receptor. The invention also provides methods of synthesis and pharmaceutical compositions comprising the aminoalkylazole compounds.

### BACKGROUND OF THE INVENTION

The histamine-3 (H₃) receptor is one of four histamine receptor subtypes (H₁-H₄), all of which are members of the G-protein-coupled receptor (GPCR) superfamily. The H₃ receptor is predominantly expressed in the central nervous system. In the brain, it is located in regions associated with learning and memory such as the cerebral cortex, hippocampus and striatum.

The H₃ receptor acts as both an auto- and hetero-receptor to regulate the release of histamine and other neurotransmitters. Within the cortex, the H₃ receptor appears to directly modify GABA release from cortical interneurons. Antagonism of the H₃ receptor produces a decrease in GABA release and disinhibition of the cortical cholinergic system, resulting in increased acetylcholine levels (Bacciottini, L. et al, Behavioral Brain Research, 124, 2001, 183-194). In addition to direct regulation of cholinergic neurotransmission, the H₃ receptor has been shown to modulate the release of dopamine, serotonin and norepinephrine (Leurs, R., et al, Trends in Pharmacological Sciences, 19,1998,177-183). Thus, H₃ receptor blockade is able to elevate concentrations of a number of neurotransmitters, including: histamine, acetylcholine, dopamine, serotonin, norepinephrine, and glutamate, and thus offers a means for targeting cognitive processes, which often rely on the integration of multiple neurotransmitter systems.

H₃ agonists have been reported to impair memory in various tasks, such as object recognition, passive avoidance (Blandina, P., et al, British Journal of Pharmacology, 119(8), 1996, 1656-1664) and social olfactory memory (Prast, H., et al, 734, 1996, 316-318), whereas H₃ antagonists have been reported to rescue impairments produced pharmacologically or genetically. Miyazaki, S., et al, Life Sciences, 61, 1997, 355-361; Meguro, K., et al, Pharmacology, Biochemistry and Behavior, 50,1995, 321-325; Fox, G. B., et. al, Beharioral Brain Research, 131, 2002,151-161; and Komater, V. A., et al, Psychopharmacology, 167, 2003, 363-372.

H₃ receptors are targets for the control of arousal and vigilance as well as for the treatment of sleep disorders because they colocalize with histaminergic neurons in brain regions that regulate the sleep-wake cycle and they modulate histamine release and levels in the CNS. Passani et al. Trends Pharmacol. Sci. 25, 618-25, 2004. The administration of selective H₃ receptor agonists, such as R-α-methylhistamine, increases sleep time and slow wave sleep in cats and rodents and produces sedation in the guinea pig, whereas H₃ antagonists such as thioperamide increase wakefulness in cats and rats and decrease slow wave sleep and REM sleep in rats. Monti et al. Eur. J. Pharmacol. 205, 283-287,1991 and Esbenshade et al. Molecular Interventions 6:77-88, 2006.

Studies on memory consolidation and spatial memory impairments, which are particularly prevelant in AD and dementia, have revealed that the H₃ antagonist thioperamide improves recall in a mouse model of premature senescence as well as in spontaneously hypertensive rat pups, and also prevents scopolamine-induced amnesia. Meguro et al. Pharmacol. Biochem. Behav. 50, 321-325, 1995 and Hancock et al. Expert Opin. Investig. Drugs 13,1237-1248, 2004. Further, H₃ receptor knockout mice are insensitive to the effects of scopolamine in an inhibitory avoidance paradigm, supporting a role for H₃ receptor modulation of cholinergic function in memory acquisition. Toyota et al. Mol. Pharmacol. 62, 389-397, 2002.

Impairments in social recognition memory are apparent in AD, but may also be relevant to social cognitive impairment in schizophrenia and ADHD. Esbenshade et al. Molecular Interventions 6:77-88, 2006. Social recognition tests have been used to show that the administration of selective histaminergic agonists enhances social memory, whereas recall is disrupted by the inhibition of histamine synthesis. Prast et al. Brain Res. 734, 316-318, 1996. In particular, thioperamide as well as several other H₃ receptor antagonists have been attributed with pro-cognitive effects. Id. In working memory impairments, prevalent in AD, ADHD, and schizophrenia, thioperamide reverses scopolamine-induced deficits. Barbier et al. Br. J. Pharmacol. 143, 649-661, 2004 and Fox et al. J. Pharmacol. Exp. Ther. 305, 897-908, 2003. Thioperamide, ciproxifan, and GT-2331, all H₃ antagonists, are also efficacious in treating impulsivity associated with ADHD in spontaneous hypertensive rat pups. Fox et al. Behav. Brain Res. 131, 151-161, 2002.

The H₃ receptor is also involved in pathological processes in the 6-OHDA (6-hydroxydopamine) lesioned rat brain, a well-characterized model of Parkinson's disease. Increased H₃ receptor mRNA expression and binding may, for example, modulate GABAergic neuronal activity in dopamine-depleted striatum. Anichtchik et al., European Journal of Neuroscience, 12 (11), 3823-3832 2000.

Methamphetamine-induced hyperlocomotor activity, a behaviorally relevant model for psychosis, can be attenuated by ciproxifan in mice (Morisset et al. J. Pharmacol. Exp. Ther. 300, 621-628, 2002), as well as by the antipsychotic drug risperidone and the H₃ receptor antagonist ABT-239. Fox et al. J. Pharmacol. Exp. Ther. 313,176-190 (2005). H₃ antagonists, such as thioperamide, have also been shown to reduce cumulative food consumption, weight gain and are suggested to have antidepressant activity. Esbenshade et al. supra and Perez-Garcia et al. Psychopharmacologia, 142(2) 215-220.1999.

Accordingly, there is significant neuroanatomical, neurochemical, pharmacological and behavioral data to support the use of H₃ receptor antagonists for improving cognitive performance in disease states such as neurodegeneration, cognitive impairment, Alzheimer's disease, Parkinson's disease, dementia, psychosis, depression, attention deficit disorder (ADD)/attention deficit hyperactivity disorder (ADHD), schizophrenia, obesity and sleep disorders.

Accordingly, compounds which are inhibitors of the H₃ receptor find use as potential therapeutic agents in the treatment of a variety of central nervous system disorders related to or affected by the H₃ receptor.

### SUMMARY OF THE INVENTION

The present invention provides an isoquinolinyl or isoindolinyl compound of formula I

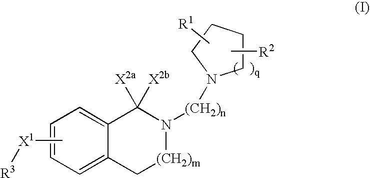

wherein
X¹ is (CR⁴R⁵)ₚ, CO or O;
X^{2a} and X^{2b} are each H or are taken together to form ═O;
m is 0,1 or 2;
n is 2, 3 or 4;
p is 0,1 or 2;
q is 1, 2 or 3;
R¹ and R² are each independently H, halogen or an alkyl or haloalkyl group each group optionally substituted;
R³ is NR⁶R⁷ or an alkyl, cycloalkyl, cycloheteroalkyl, aryl or heteroaryl group each group optionally substituted with the proviso that when X¹ is O then R³ must be other than NR⁶R⁷; when X^{2a} and X^{2b} are taken together to form ═O and p is 0, then R³ is not quinoxalinyl-2(1H)-one or an optionally substituted 1,3,4-oxadiazole; and when X^{2a} and X^{2b} are H and p is 0, then R³ is not an optionally substituted 1,2,4-triazol-5(4H)-one
R⁴ and R⁵ are each independently H or an optionally substituted alkyl or cycloalkyl group; and
R⁶ and R⁷ each independently H or an alkyl, alkenyl, alkoxy, cycloalkyl, cycloheteroalkyl, aryl or heteroaryl group each group optionally substituted or R⁶ and R⁷ may be taken together with the atom to which they are attached to form an optionally substituted 4- to 7-membered ring optionally containing one or two additional heteroatoms selected from N, O or S or an optionally substituted fused bicyclic or tricyclic 9- to 15-membered aromatic ring system optionally containing one to three additional heteroatoms selected from N, O or S; or
   a stereoisomer thereof or a pharmaceutically acceptable salt thereof.

The present invention also provides methods and compositions useful for the therapeutic treatment of central nervous system disorders related to or affected by the Histamine-3 receptor.

### DETAILED DESCRIPTION OF THE INVENTION

Alzheimer's disease (AD) is characterized by a progressive loss of memory and cognitive function and is the most common cause of dementia in the elderly. AD is believed to affect approximately 15-20 million people worldwide. The goal of treatment in AD, in addition to reversing the disease process, is to improve or at least slow the loss of memory and cognition and to maintain independent function in patients with mild to moderate disease. AD is characterized by numerous deficits in neurotransmitter function (Möller, H-J., European Neuropsychopharmacology, 9, 1999, S53-S59), further a postmortem study in humans suggests that a decrease in brain histamine levels may contribute to the cognitive decline associated with AD, directly or through the cholinergic system (Panula, P., et al, Neuroscience, 82, 1998, 993-997). Histamine-3 (H₃) receptor antagonists have been reported to rescue impairments produced pharmacologically or genetically (Miyazaki, S., et al, Life Sciences, 61, 1997, 355-361; Meguro, K., et al, Pharmacology, Biochemistry and Behavior, 50, 1995, 321-325; Fox, G. B., et. al, Beharioral Brain Research, 131, 2002, 151-161; and Komater, V. A., et al, Psychopharmacology, 167, 2003, 363-372). Neuroanatomical, neurochemical, pharmacological and behavioral data support the belief that H₃ receptor antagonists may improve cognitive performance in disease states such as mild cognitive impairment and Alzheimer's disease and may have therapeutic value in the treatment of attention deficit disorder (ADD)/attention deficit hyperactivity disorder (ADHD), schizophrenia, particularly cognitive dysfunction in schizophrenia, dementia, psychosis, depression, Parkinson's disease, obesity, eating disorders, sleep disorders and neuropathic pain. To that end, compounds which inhibit the H₃ receptor and act as H₃ antagonists are earnestly sought.

Surprisingly it has now been found that isoquinolinone and isoquinolinone compounds of formula I demonstrate H₃ affinity along with significant sub-type selectivity and function as H₃ antagonists. Advantageously, said formula I compounds are effective therapeutic agents for the treatment of central nervous system (CNS) disorders associated with or affected by the H₃ receptor. Accordingly, the present invention provides a isoquinolinone or isoquinolinone compound of formula I

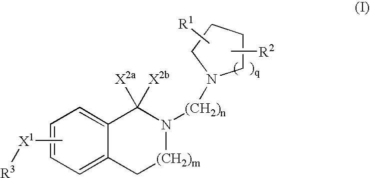

wherein
X¹ is (CR⁴R⁵)ₚ, CO or O;
X^{2a} and X^{2b} are each H or are taken together to form ═O;
m is 0, 1 or 2;
n is 2, 3 or 4;
p is 0, 1 or 2;
q is 1, 2 or 3;
R¹ and R² are each independently H, halogen or an alkyl or haloalkyl group each group optionally substituted;
R³ is NR⁶R⁷ or an alkyl, cycloalkyl, cycloheteroalkyl, aryl or heteroaryl group each group optionally substituted with the proviso that when X¹ is O then R³ must be other than NR⁶R⁷; when X^{2a} and X^{2b} are taken together to form ═O and p is 0, then R³ is not quinoxalinyl-2(1H)-one or an optionally substituted 1,3,4-oxadiazole; and when X^{2a} and X^{2b} are H and p is 0, then R³ is not an optionally substituted 1,2,4-triazol-5(4H)-one;
R⁴ and R⁵ are each independently H or an optionally substituted alkyl or cycloalkyl group; and

R⁶ and R⁷ each independently H or an alkyl, alkenyl, alkoxy, cycloalkyl, cycloheteroalkyl, aryl or heteroaryl group each group optionally substituted or R⁶ and R⁷ may be taken together with the atom to which they are attached to form an optionally substituted 4- to 7-membered ring optionally containing one or two additional heteroatoms selected from N, O or S or an optionally substituted fused bicyclic or tricyclic 9- to 15-membered aromatic ring system optionally containing one to three additional heteroatoms selected from N, O or S; or

a stereoisomer thereof or a pharmaceutically acceptable salt thereof.

It is understood that the claims encompass all possible stereoisomers and prodrugs.

Another aspect of the invention provides a method for the treatment of a cognitive disorder related to or affected by the Histamine-3 (H₃) receptor in a patient in need thereof which comprises providing to said patient a therapeutically effective amount of a compound of formula I or any other embodiment thereof described herein. In a more particular embodiment, said disorder is a neurodegenerative disorder. More particular still, said disorder is mild cognitive impairment (MCI), dementia, delirium, amnestic disorder, Alzheimer's disease (AD), Parkinson's disease (PD), Huntington's disease (HD), memory disorder, memory deficits associated with depression, schizophrenia, a psychotic disorder, paranoia, mano-depressive illness, attention deficit disorder (ADD), attention deficit hyperactivity disorder (ADHD), dyslexia, developmental disorders, Down's syndrome, Fragile X syndrome, loss of executive function, loss of learned information, vascular dementia, cognitive decline, neurodegenerative disorder, HIV-induced dimentia, head trauma, Pick's disease, Creutzfeldt-Jakob disease, Body dementia, vascular dementia, surgical procedure-induced cognitive dysfunction, traumatic brain injury or stroke. In another more particular embodiment, said disorder is selected from the group consisting of: Alzheimer's disease, attention deficit disorder, schizophrenia; Parkinsons' disease, frontal temporal dementia or depression.

Another aspect of the invention provides a method for the inhibition of an H₃ receptor comprising contacting said receptor with an effective amount of a compound of formula I or any other embodiment thereof described herein.

An additional aspect of the invention provides a pharmaceutical composition which comprises a pharmaceutically acceptable carrier and an effective amount of a compound of formula I or any other embodiment thereof described herein.

“Treating” or “treatment” of a disease in a subject refers to inhibiting the disease or arresting its development; ameliorating symptoms of the disease; or causing regression of the disease.

Additionally, the compound of the invention may be used in the prevention of a disease described herein.

A “cognitive disease,” “cognitive dysfunction,” or “cognition-related disorder” is a disease or disorder affecting mental processes such as memory, attention, perception, action, problem solving and mental imagery. Cognitive dysfunction generally originates in the central nervous system and can be influenced or derived from neurodegeneration. Particular cognition-related disorders (e.g., cognitive dysfunction) include, without limitation, mild cognitive impairment (MCI), dementia, delirium, amnestic disorder, Alzheimer's disease, Parkinson's disease, Huntington's disease, memory disorders including memory deficits associated with depression, senile dementia, dementia of Alzheimer's disease, cognitive deficits or cognitive dysfunction associated with neurological conditions including, for example, Parkinson's disease (PD), Huntington's disease (HD), Alzheimer's disease, depression and schizophrenia (and other psychotic disorders such as paranoia and mano-depressive illness); cognitive dysfunction in schizophrenia, disorders of attention and learning such as attention deficit disorder (ADD), attention deficit hyperactivity disorder (ADHD), and dyslexia, cognitive dysfunction associated with developmental disorders such as Down's syndrome and Fragile X syndrome, loss of executive function, loss of learned information, vascular dementia, schizophrenia, cognitive decline, neurodegenerative disorder, and other dementias, for example, due to HIV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the embodiments described herein.

The term “H₃ antagonist” or “H₃ inhibitor” as used herein refers to a composition that reduces activity of the H₃ receptor. H₃ antagonists described herein can either reduce constitutive H₃ activity independent of agonist interaction (i.e. function as an inverse agonist) or reduce H₃ agonist-mediated activity.

An optionally substituted moiety may be substituted with one or more substituents, which may be the same or different. The substituent groups, which are optionally present, may be one or more of those customarily employed in the development of pharmaceutical compounds or the modification of such compounds to influence their structure/activity, persistence, absorption, stability or other beneficial property. Specific examples of such substituents include halogen atoms, nitro, cyano, thiocyanato, cyanato, hydroxyl, alkyl, haloalkyl, alkoxy, haloalkoxy, amino, alkylamino, dialkylamino, formyl, alkoxycarbonyl, carboxyl, alkanoyl, alkylthio, alkylsuphinyl, alkylsulphonyl, carbamoyl, alkylamido, phenyl, phenoxy, benzyl, benzyloxy, heterocyclyl or cycloalkyl groups, preferably halogen atoms or lower alkyl or lower alkoxy groups. Additional examples of optionally substituted groups include (3-phenylpropylthio)methyl and 2-(2-phenoxyethylamino)ethyl. Unless otherwise specified, typically, 0 to 4, 0 to 3, 0 to 2 or 0 to 1 substituents may be present. Optionally substituted groups may themselves be substituted with up to three levels of substitution.

Preferably, optionally substituted refers to the replacement of 0 to 4, 0 to 3, 0 to 2 or 0 to 1 hydrogen atoms with 0 to 4, 0 to 3, 0 to 2 or 0 to 1 groups selected from C₁-C₆ alkyl, C₃-C₆ cycloakyl, C₂-C₆ alkenyl, C₂-C₆ alkynyl, halo, nitro, cyano, hydroxy, C₆-C₁₀ aryl, a 3-10 membered heterocyclyl ring, a 5-10 membered heteroaryl ring, —N(R^{a})₂, —C(O)R^{b}, —OR^{c} and —S(O)ₚR_{d}; wherein each R^{a} is independently H, C₁-C₄ alkyl, 'CHO, —C(O)(C₁-C₄ alkyl), or —CO₂(C₁-C₄ alkyl); each R^{b} is independently H, —OH, —O(C₁-C₄), C₁-C₄ alkyl, —NH₂, —NH(C₁-C₄ alkyl), or —N(C₁-C₄ alkyl)₂; each R^{c} is independently H, C₁-C₄ alkyl optionally substituted with halo, —CHO or —C(O)(C₁-C₄ alkyl); each R^{d} is independently C₁-C₄ alkyl, or —OH; and p is 0, 1 or 2. A suitable group of substituents is CN, OH, —NH₂, —NH(C₁-C₄ alkyl), or —N(C₁-C₄ alkyl)₂;, halogen, phenyl, carbamoyl, carbonyl, alkoxy or aryloxy.

As used herein, the term alkyl refers to a linear or branched alkyl moiety containing up to 12 carbon atoms, e.g. up to 10 carbon atoms, preferably up to 6 carbon atoms, more preferably up to 4 carbon atoms Examples of saturated hydrocarbon alkyl moieties include, but are not limited to, chemical groups such as methyl, ethyl, n-propyl, isopropyl, n-butyl, tert-butyl, isobutyl, sec-butyl; higher homologs such as n-pentyl, n-hexyl, and the like.

As used herein, the term haloalkyl designates a CₙH₂ₙ₊₁ group having from one to 2n+1 halogen atoms which may be the same or different. Examples of haloalkyl groups include CF₃, CH₂Cl, C₂H₃BrCl, C₃H₅F₂, or the like.

The term halogen, as used herein, designates fluorine, chlorine, bromine, and iodine.

The term alkenyl, as used herein, refers to either a (C₂-C₁₀) straight chain or (C₃-C₁₀) branched-chain monovalent hydrocarbon moiety containing at least one double bond. The alkenyl is suitably a (C₂-C₈), (C₂-C₆), (C₂-C₄) or (C₂-C₃) moiety. Such hydrocarbon alkenyl moieties may be mono or polyunsaturated, and may exist in the E or Z configurations. The compounds of this invention are meant to include all possible E and Z configurations. Examples of mono or polyunsaturated hydrocarbon alkenyl moieties include, but are not limited to, chemical groups such as vinyl, 2-propenyl, isopropenyl, crotyl, 2-isopentenyl, butadienyl, 2-(butadienyl), 2,4-pentadienyl, 3-(1,4-pentadienyl), and higher homologs, isomers, or the like.

The term alkynyl, as used in the specification and claims, designates either a (C₂-C₁₀) straight chain or (C₃-C₁₀) branched chain monovalent hydrocarbon moiety having at least one triple bond. The alkynyl is suitably a (C₂-C₈), (C₂-C₆), (C₂-C₄) or (C₂-C₃) moiety. Such hydrocarbon alkynyl moieties may be mono or polyunsaturated, and may exist in the E or Z configurations. The compounds of this invention are meant to include all possible E and Z configurations. Examples of mono or polyunsaturated hydrocarbon alkynyl moieties include, but are not limited to, propynyl, butynyl, 1,3-butadiynyl, pentynyl, hexynyl, or the like.

The term cycloalkyl, as used herein, refers to a monocyclic, bicyclic, tricyclic, fused, bridged, or spiro monovalent saturated hydrocarbon moiety of 3-10 carbon atoms. The cycloalkyl is suitably a (C₃-C₈)or a (C₃-C₆) moiety. Examples of cycloalkyl moieties include, but are not limited to, chemical groups such as cyclopropyl, cyclobutyl, cyclopentyl, cyclohexyl, cycloheptyl, norbornyl, adamantyl, spiro[4.5]decanyl, or the like.

The term cycloheteroalkyl, as used herein, designates one or more (fused if more than one) 5-7 membered ring systems containing 1, 2 or 3 heteroatoms, which may be the same or different, selected from N, O or S and optionally containing at least one double bond. Exemplary of the cycloheteroalkyl ring systems included in the term as designated herein are the following rings wherein X₁ is NR′, O or S and R′ is H or an optional substituent as defined hereinabove (when there are two X₁ groups they may be the same or different).

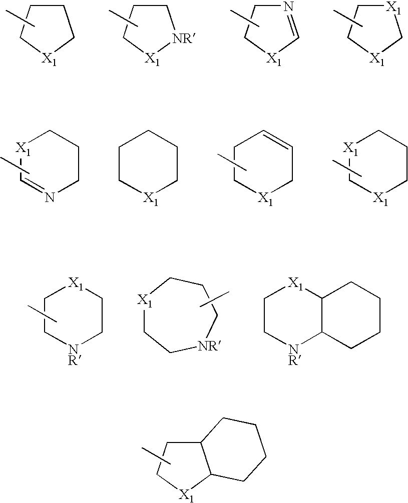

The term aryl, as used herein, refers to an aromatic carbocyclic moiety of up to 20 carbon atoms, which may be a single ring (monocyclic) or multiple rings (up to three rings) fused together. Examples of aryl moieties include, but are not limited to, chemical groups such as phenyl, 1-naphthyl, 2-naphthyl, anthryl, or the like. Aryl also includes polycyclic rings containing heterocyclic rings that are appended through the aromatic carbocyclic ring (e.g. 1,3-benzodioxol-5-yl).

The term heteroaryl as used herein designates an aromatic heterocyclic ring system, which may be a single ring (monocyclic) or multiple rings (up to three rings) fused together. The rings may contain from one to four hetero atoms selected from nitrogen, oxygen, or sulfur, which may be the same or different, wherein the nitrogen or sulfur atoms are optionally oxidized, or the nitrogen atom is optionally quarternized. Examples of heteroaryl moieties include, but are not limited to, heterocycles such as furan, thiophene, pyrrole, pyrazole, imidazole, oxazole, isoxazole, thiazole, isothiazole, oxadiazole, triazole, pyridine, pyrimidine, pyrazine, pyridazine, benzimidazole, benzoxazole, benzisoxazole, benzothiazole, benzofuran, benzothiophene, thianthrene, dibenzofuran, dibenzothiophene, indole, indazole, azaindole, azaindazole, quinoline, isoquinoline, quinazoline, quinoxaline, purine, or the like.

As used herein: EDC designates 1-(3-dimethylaminopropyl)-3-ethylcarbo-diimide hydrochloride; HOBt designates 1-hydroxybenzotriazole; DIPEA designates diisopropylethylamine; Burgess Reagent designates (methoxycarbonylsulfamoyl)-triethylammonium hydroxide, inner salt; and DBU designates 1,8-diazabicyclo[5.4.0]-undec-7-ene.

Unless otherwise stated, structures depicted herein are also meant to include all stereochemical forms of the structure; i.e., the R and S configurations for each asymmetric center and geometric isomers around a double bond (E and Z). Therefore, single stereochemical isomers as well as enantiomeric and diastereomeric mixtures of the present compounds are within the scope of the invention. Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are within the scope of this invention.

Unless indicated otherwise, the nomenclature of substituents that are not explicitly defined herein are arrived at by naming the terminal portion of the functionality followed by the adjacent functionality toward the point of attachment.

The compounds of the present invention may be converted to salts, in particular pharmaceutically acceptable salts using art recognized procedures. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Salts which are unsuitable for pharmaceutical uses but which can be employed, for example, for the isolation or purification of free compounds or their pharmaceutically acceptable salts, are also included. The term “pharmaceutically acceptable salt”, as used herein, refers to salts derived from organic and inorganic acids such as, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids when a compound of this invention contains a basic moiety. Salts may also be formed from organic and inorganic bases, preferably alkali metal salts, for example, sodium, lithium, or potassium, when a compound of this invention contains a carboxylate or phenolic moiety, or similar moiety capable of forming base addition salts.

Compounds of the invention include esters, carbamates or other conventional prodrug forms, which in general, are functional derivatives of the compounds of the invention and which are readily converted to the inventive active moiety in vivo. Correspondingly, the method of the invention embraces the treatment of the various conditions described hereinabove with a compound of formula I or with a compound which is not specifically disclosed but which, upon administration, converts to a compound of formula I in vivo. Also included are metabolites of the compounds of the present invention defined as active species produced upon introduction of these compounds into a biological system.

Preferred compounds of the invention are those compounds of formula I wherein X is (CR⁴R⁵)p or O. Another group of preferred compounds is those formula I compounds wherein R¹ and R² are each independently H or methyl.

Another group of preferred compounds is those formula I compounds wherein X¹ is (CR⁴R⁵)ₚ and p is 0. Another group of preferred compounds is those formula I compounds wherein X^{2a} and X^{2b} are each H. Another group of preferred compounds is those formula I compounds wherein X^{2a} and X^{2b} are taken together to form ═O; or a stereoisomer, tautomer or pharmaceutically acceptable salt thereof.

In a more particular embodiment of the compound of formula 1, R³is not 2-((3-phenylpropylthio)methyl)-1,3,4-oxadiazole or 1-(2-(2-phenoxyethylamino)ethyl)-1H-1,2,4-triazol-5(4H)-one. In another embodiment, R³is not an optionally substituted group having the following structure:

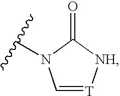

wherein T is N or CH.

Another group of preferred compounds is those formula I compounds wherein R³ is an optionally substituted aminocarbonylphenyl or cycloheteroalkylcarbonylphenyl group. In a particular embodiment, when R³ is an aminocarbonylphenyl group, the optional substitution at the amino group is alkyl or cycloalkyl and the optional substitution at the phenyl group is halo.

Another group of preferred compounds is those formula I compounds wherein R³ is selected from the group consisting of phenyl, halophenyl, dihalophenyl, perhaloalkoxyphenyl, cyanophenyl, perhaloalkylphenyl, alkoxyphenyl, alkoxycarbonylphenyl, heteroaryl, cycloheteroalkylcarbonyl, cycloheteroalkylcarbonylphenyl, cyanoheteroaryl, carboxyphenyl, cycloalkylaminocarbonylphenyl, N,N-dialkylaminocarbonylphenyl, alkylaminocarbonylphenyl, alkycycloheteroalkylcarbonylphenyl, aminocarbonylphenyl, alkylaminocarbonylheteroaryl, cycloalkylcarbonylphenyl, cyanophenylalkoxy and dihydroisoquinolinone; or a stereoisomer, tautomer or pharmaceutically acceptable salt thereof.

Another group of preferred compounds is those formula I compounds wherein q is 1 or 2.

In one embodiment of the invention, preferred compounds of formula I are those compounds having the structure of formula Ia

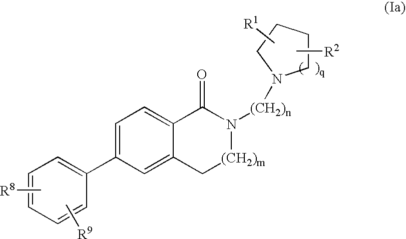

wherein
R¹, R², m, n and q are as described for formula I;
R⁸ and R⁹ are each independently H, halogen, CN, CONR¹⁰R¹¹, OR¹², CO₂R¹², COR¹², or an alkyl, haloalkyl or cycloalkyl group each group optionally substituted;
R¹⁰ and R¹¹ are each independently H or an alkyl, haloalkyl, cycloalkyl, aryl or heteroaryl group each group optionally substituted or R¹⁰ and R¹¹ may be taken together with the atom to which they are attached to form an optionally substituted 4- to 7-membered ring optionally containing one or two additional heteroatoms selected from N, O or S; and
R¹² is H or an alkyl, haloalkyl, cycloalkyl, alkenyl, alkynyl, aryl or heteroaryl group each group optionally substituted; or
   a stereoisomer thereof or a pharmaceutically acceptable salt thereof.

More preferred compounds of the invention are those compounds of formula I wherein m is 0 or 1, q is 1 or 2 and R¹ and R² each independently H or methyl. Another group of more preferred compounds is those compounds of formula la wherein n is 2 or 3; q is 1 or 2 and m is 0 or 1. A further group of more preferred compounds are those compounds of formula la wherein R⁸ is H or halogen and R⁹ is CONR¹⁰R¹¹.

Among the preferred compounds of the invention are:
6-(4-fluorophenyl)-2-(2-pyrrolidin-1-ylethyl)-3,4-dihydroisoquinolin-1(2H)-one;
6-(3,5-difluorophenyl)-2-(2-pyrrolidin-1-ylethyl)-3,4-dihydroisoquinolin-1(2H)-one;
6-(2,4-difluorophenyl)-2-(2-pyrrolidin-1-ylethyl)-3,4-dihydroisoquinolin-1(2H)-one;
6-(2-fluorophenyl)-2-(2-pyrrolidin-1-ylethyl)-3,4-dihydroisoquinolin-1(2H)-one;
2-(2-pyrrolidin-1-ylethyl)-6-[3-(trifluoromethoxy)phenyl]-3,4-dihydroisoquinolin-1(2H)-one;
2-(2-pyrrolidin-1-ylethyl)-6-[4-(trifluoromethoxy)phenyl]-3,4-dihydroisoquinolin-1(2H)-one;
3-[1-oxo-2-(2-pyrrolidin-1-ylethyl)-1,2,3,4-tetrahydroisoquinolin-6-yl]benzonitrile;
6-phenyl-2-(2-pyrrolidin-1-ylethyl)-3,4-dihydroisoquinolin-1(2H)-one;
6-(3,4-difluorophenyl)-2-(2-pyrrolidin-1-ylethyl)-3,4-dihydroisoquinolin-1(2H)-one;
6-(3-fluorophenyl)-2-(2-pyrrolidin-1-ylethyl)-3,4-dihydroisoquinolin-1(2H)-one;
4-[1-oxo-2-(2-pyrrolidin-1-ylethyl)-1,2,3,4-tetrahydroisoquinolin-6-yl]benzonitrile;
2-(2-pyrrolidin-1-ylethyl)-6-[3-(trifluoromethyl)phenyl]-3,4-dihydroisoquinolin-1(2H)-one;
6-(1,3-benzodioxol-5-yl)-2-(2-pyrrolidin-1-ylethyl)-3,4-dihydroisoquinolin-1(2H)-one;
2-(2-pyrrolidin-1-ylethyl)-6-[4-(trifluoromethyl)phenyl]-3,4-dihydroisoquinolin-1(2H)-one;
6-(4-methoxyphenyl)-2-(2-pyrrolidin-1-ylethyl)-3,4-dihydroisoquinolin-1(2H)-one;
methyl 4-(1-oxo-2-(2-(pyrrolidin-1-yl)ethyl)-1,2,3,4-tetrahydroisoquinolin-6-yl)benzoate;
methyl 4-(2-{2-[(2R)-2-methylpyrrolidin-1-yl]ethyl}-1-oxo-1,2,3,4-tetrahydroiso-quinolin-6-yl)benzoate;
4-(2-{2-[(2R)-2-methylpyrrolidin-1-yl]ethyl}-1-oxo-1,2,3,4-tetrahydroisoquinolin-7-yl)benzonitrile;
3-(2-{2-[(2R)-2-methylpyrrolidin-1-yl]ethyl}-1-oxo-1,2,3,4-tetrahydroisoquinolin-5-yl)benzonitrile;
4-(2-{2-[(2R)-2-methylpyrrolidin-1-yl]ethyl}-1-oxo-1,2,3,4-tetrahydroisoquinolin-5-yl)benzonitrile;
4-[1-oxo-2-(3-pyrrolidin-1-ylpropyl)-1,2,3,4-tetrahydroisoquinolin-6-yl]benzonitrile;
3-[1-oxo-2-(3-pyrrolidin-1-ylpropyl)-1,2,3,4-tetrahydroisoquinolin-6-yl]benzonitrile;
6-pyridin-4-yl-2-(2-pyrrolidin-1-ylethyl)-3,4-dihydroisoquinolin-1(2H)-one;
1-[1-oxo-2-(2-pyrrolidin-1-ylethyl)-1,2,3,4-tetrahydroisoquinolin-6-yl]-1H-indole-5-carbonitrile;
6-(pyrrolidin-1-ylcarbonyl)-2-(2-pyrrolidin-1-ylethyl)-3,4-dihydroisoquinolin-1(2H)-one;
6-(4-fluorophenyl)-2-{2-[(2S)-2-methylpyrrolidin-1-yl]ethyl}-3,4-dihydroisoquinolin-1(2H)-one;
6-(4-fluorophenyl)-2-{2-[(2R)-2-methylpyrrolidin-1-yl]ethyl}-3,4-dihydroisoquinolin-1(2H)-one;
4-(2-{2-[(2S)-2-methylpyrrolidin-1-yl]ethyl}-1-oxo-1,2,3,4-tetrahydroisoquinolin-6-yl)benzonitrile;
4-(2-{2-[(2R)-2-methylpyrrolidin-1-yl]ethyl}-1-oxo-1,2,3,4-tetrahydroisoquinolin-6-yl)benzonitrile;
6-{[1-oxo-2-(2-pyrrolidin-1-ylethyl)-1,2,3,4-tetrahydroisoquinolin-6-yl]oxy}nicotinonitrile;
6-[(2-{2-[(2R)-2-methylpyrrolidin-1-yl]ethyl}-1-oxo-1,2,3,4-tetrahydroisoquinolin-6-yl)oxy]nicotinonitrile;
4-[(2-{2-[(2R)-2-methylpyrrolidin-1-yl]ethyl}-1-oxo-1,2,3,4-tetrahydroisoquinolin-6-yl)oxy]benzonitrile;
4-{[1-oxo-2-(2-pyrrolidin-1-ylethyl)-1,2,3,4-tetrahydroisoquinolin-6-yl]oxy}benzonitrile;
5-[(2-{2-[(2R)-2-methylpyrrolidin-1-yl]ethyl}-1-oxo-1,2,3,4-tetrahydroisoquinolin-6-yl)oxy]pyridine-2-carbonitrile;
5-{[1-oxo-2-(2-pyrrolidin-1-ylethyl)-1,2,3,4-tetrahydroisoquinolin-6-yl]oxy}pyridine-2-carbonitrile;
6-[4-(pyrrolidin-1-ylcarbonyl)phenyl]-2-(2-pyrrolidin-1-ylethyl)-3,4-dihydroisoquinolin-1(2H)-one;
N-cyclopentyl-4-[1-oxo-2-(2-pyrrolidin-1-ylethyl)-1,2,3,4-tetrahydroisoquinolin-6-yl]benzamide;
N,N-dimethyl-4-[1-oxo-2-(2-pyrrolidin-1-ylethyl)-1,2,3,4-tetrahydroisoquinolin-6-yl]benzamide;
N-cyclopropyl-4-[1-oxo-2-(2-pyrrolidin-1-ylethyl)-1,2,3,4-tetrahydroisoquinolin-6-yl]benzamide;
N-ethyl-4-[1-oxo-2-(2-pyrrolidin-1-ylethyl)-1,2,3,4-tetrahydroisoquinolin-6-yl]benzamide;
N-methyl-4-[1-oxo-2-(2-pyrrolidin-1-ylethyl)-1,2,3,4-tetrahydroisoquinolin-6-yl]benzamide;
N-(cyclopropylmethyl)-4-[1-oxo-2-(2-pyrrolidin-1-ylethyl)-1,2,3,4-tetrahydroiso-quinolin-6-yl]benzamide;
N-isopropyl-4-[1-oxo-2-(2-pyrrolidin-1-ylethyl)-1,2,3,4-tetrahydroisoquinolin-6-yl]benzamide;
N,N-diethyl-4-[1-oxo-2-(2-pyrrolidin-1-ylethyl)-1,2,3,4-tetrahydroisoquinolin-6-yl]benzamide;
N-cyclobutyl-4-[1-oxo-2-(2-pyrrolidin-1-ylethyl)-1,2,3,4-tetrahydroisoquinolin-6-yl ]benzamide;
6-[4-(azetidin-1-ylcarbonyl)phenyl]-2-(2-pyrrolidin-1-ylethyl)-3,4-dihydroisoquinolin-1(2H)-one;
N,N-diethyl-4-(2-{2-[(2R)-2-methylpyrrolidin-1-yl]ethyl}-1-oxo-1,2,3,4-tetrahydroisoquinolin-6-yl)benzamide;
2-{2-[(2R)-2-methylpyrrolidin-1-yl]ethyl}-6-[4-(pyrrolidin-1-ylcarbonyl)phenyl]-3,4-dihydroisoquinolin-1(2H)-one;
4-[1-oxo-2-(2-pyrrolidin-1-ylethyl)-1,2,3,4-tetrahydroisoquinolin-6-yl]benzamide;
N-(2-fluoroethyl)-4-[1-oxo-2-(2-pyrrolidin-1-ylethyl)-1,2,3,4-tetrahydroisoquinolin-6-yl]benzamide;
N-(2-methoxyethyl)-4-[1-oxo-2-(2-pyrrolidin-1-ylethyl)-1,2,3,4-tetrahydroisoquinolin-6-yl]benzamide;
N-(2-isopropoxyethyl)-4-[1-oxo-2-(2-pyrrolidin-1-ylethyl)-1,2,3,4-tetrahydroisoquinolin-6-yl]benzamide;
4-[1-oxo-2-(2-pyrrolidin-1-ylethyl)-1,2,3,4-tetrahydroisoquinolin-6-yl]-N-(2-phenoxyethyl)-benzamide;
N-(2-ethoxyethyl)-4-[1-oxo-2-(2-pyrrolidin-1-ylethyl)-1,2,3,4-tetrahydroisoquinolin-6-yl]benzamide;
N-(cyclopropylmethyl)-4-(2-{2-[(2R)-2-methylpyrrolidin-1-yl]ethyl}-1-oxo-1,2,3,4-tetrahydroisoquinolin-6-yl)benzamide;
N-cyclobutyl-4-(2-{2-[(2R)-2-methylpyrrolidin-1-yl]ethyl}-1-oxo-1,2,3,4-tetrahydroisoquinolin-6-yl)benzamide;
N-ethyl-4-(2-{2-[(2R)-2-methylpyrrolidin-1-yl]ethyl}-1-oxo-1,2,3,4-tetrahydroisoquinolin-6-yl)benzamide;
N-cyclopropyl-4-(2-{2-[(2R)-2-methylpyrrolidin-1-yl]ethyl}-1-oxo-1,2,3,4-tetrahydroisoquinolin-6-yl)benzamide;
N-isopropyl-4-(2-{2-[(2R)-2-methylpyrrolidin-1-yl]ethyl}-1-oxo-1,2,3,4-tetrahydroisoquinolin-6-yl)benzamide;
N-methyl-4-(2-{2-[(2R)-2-methylpyrrolidin-1-yl]ethyl}-1-oxo-1,2,3,4-tetrahydroisoquinolin-6-yl)benzamide;
6-[4-(piperidin-1-ylcarbonyl)phenyl]-2-(2-pyrrolidin-1-ylethyl)-3,4-dihydroisoquinolin-1(2H)-one;
N-cyclopentyl-4-(2-{2-[(2R)-2-methylpyrrolidin-1-yl]ethyl}-1-oxo-1,2,3,4-tetrahydroisoquinolin-6-yl)benzamide;
6-(4-{[(2S)-2-methylpyrrolidin-1-yl]carbonyl}phenyl)-2-(2-pyrrolidin-1-ylethyl)-3,4-dihydroisoquinolin-1(2H)-one;
6-(4-{[(2R)-2-methylpyrrolidin-1-yl]carbonyl}phenyl)-2-(2-pyrrolidin-1-ylethyl)-3,4-dihydroisoquinolin-1(2H)-one;
N-methyl-4-(2-{2-[(2R)-2-methylpyrrolidin-1-yl]ethyl}-1-oxo-1,2,3,4-tetrahydroiso-quinolin-7-yl)benzamide;
N-ethyl-4-(2-{2-[(2R)-2-methylpyrrolidin-1-yl]ethyl}-1-oxo-1,2,3,4-tetrahydroiso-quinolin-7-yl)benzamide;
N-isopropyl-4-(2-{2-[(2R)-2-methylpyrrolidin-1-yl]ethyl}-1-oxo-1,2,3,4-tetrahydroiso-quinolin-7-yl)benzamide;
2-{2-[(2R)-2-methylpyrrolidin-1-yl]ethyl}-7-[4-(pyrrolidin-1-ylcarbonyl)phenyl]-3,4-dihydroisoquinolin-1(2H)-one;
4-{[1-oxo-2-(2-pyrrolidin-1-ylethyl)-1,2,3,4-tetrahydroisoquinolin-6-yl]oxy}benzamide
N-methyl-4-{[1-oxo-2-(2-pyrrolidin-1-ylethyl)-1,2,3,4-tetrahydroisoquinolin-6-yl]oxy}benzamide;
N-ethyl-4-{[1-oxo-2-(2-pyrrolidin-1-ylethyl)-1,2,3,4-tetrahydroisoquinolin-6-yl]oxy}benzamide;
N-isopropyl-4-{[1-oxo-2-(2-pyrrolidin-1-ylethyl)-1,2,3,4-tetrahydroisoquinolin-6-yl]oxy}benzamide;
N,N-dimethyl-4-{[1-oxo-2-(2-pyrrolidin-1-ylethyl)-1,2,3,4-tetrahydroisoquinolin-6-yl]oxy}benzamide;
N,N-diethyl-4-{[1-oxo-2-(2-pyrrolidin-1-ylethyl)-1,2,3,4-tetrahydroisoquinolin-6-yl]oxy}benzamide;
N-cyclobutyl-4-{[1-oxo-2-(2-pyrrolidin-1-ylethyl)-1,2,3,4-tetrahydroisoquinolin-6-yl]oxy}benzamide;
6-[4-(pyrrolidin-1-ylcarbonyl)phenoxy]-2-(2-pyrrolidin-1-ylethyl)-3,4-dihydroiso-quinolin-1(2H)-one;
N-cyclopropyl-4-{[1-oxo-2-(2-pyrrolidin-1-ylethyl)-1,2,3,4-tetrahydroisoquinolin-6-yl]oxy}benzamid;e
N-methyl-6-{[1-oxo-2-(2-pyrrolidin-1-ylethyl)-1,2,3,4-tetrahydroisoquinolin-6-yl]oxy}nicotinamide;
N-methoxy-N-methyl-4-(1-oxo-2-(2-(pyrrolidin-1-yl)ethyl)-1,2,3,4-tetrahydroisoquinolin-6-yl)benzamide;
6-[4-(cyclopropylcarbonyl)phenyl]-2-(2-pyrrolidin-1-ylethyl)-3,4-dihydroisoquinolin-1(2H)-one;
6-(1H-benzimidazol-1-yl)-2-(2-pyrrolidin-1-ylethyl)-3,4-dihydroisoquinolin-1(2H)-one;
5-(1H-benzimidazol-1-ylmethyl)-2-(2-pyrrolidin-1-ylethyl)isoindolin-1-one;
6-(4-fluorophenyl)-2-(2-piperidin-1-ylethyl)-3,4-dihydroiso-quinolin-1(2H)-one;
4-[1-oxo-2-(3-piperidin-1-ylpropyl)-1,2,3,4-tetrahydroisoquinolin-6-yl]benzonitrile;
2-(2-azepan-1-ylethyl)-6-(4-fluorophenyl)-3,4-dihydroisoquinolin-1(2H)-one;
4-(2-{2-[(2R)-2-methylpyrrolidin-1-yl]ethyl}-1-oxo-2,3-dihydro-1H-isoindol-5-yl)benzonitrile;
4-{[1-oxo-2-(2-pyrrolidin-1-ylethyl)-1,2,3,4-tetrahydroisoquinolin-6-yl]oxy}benzamide;
4-[1-oxo-2-(2-pyrrolidin-1-ylethyl)-1,2,3,4-tetrahydroisoquinolin-6-yl]benzoic acid;
4-{[1-oxo-2-(2-pyrrolidin-1-ylethyl)-1,2,3,4-tetrahydroisoquinolin-6-yl]oxy}benzoic acid;
4-{[(2-{2-[(2R)-2-methylpyrrolidin-1-yl]ethyl}-1-oxo-1,2,3,4-tetrahydroisoquinolin-6-yl)oxy]methyl}benzonitrile;
4-[1-oxo-2-(2-pyrrolidin-1-ylethyl)-1,2,3,4-tetrahydroisoquinolin-6-yl]-N-(2-thienylmethyl)benzamide;
6-[4-(morpholin-4-ylcarbonyl)phenyl]-2-(2-pyrrolidin-1-ylethyl)-3,4-dihydroisoquinolin-1(2H)-one;
N-(2-chloroethyl)-4-[1-oxo-2-(2-pyrrolidin-1-ylethyl)-1,2,3,4-tetrahydroisoquinolin-6-yl]benzamide;
N-ethyl-N-methyl-4-[1-oxo-2-(2-pyrrolidin-1-ylethyl)-1,2,3,4-tetrahydroisoquinolin-6-yl]benzamide;
N-(2-furylmethyl)-4-[1-oxo-2-(2-pyrrolidin-1-ylethyl)-1,2,3,4-tetrahydroisoquinolin-6-yl]benzamide;
N-[(1S)-2-methoxy-1-methylethyl]-4-[1-oxo-2-(2-pyrrolidin-1-ylethyl)-1,2,3,4-tetrahydroisoquinolin-6-yl]benzamide;
6-{4-[(3-methoxypyrrolidin-1-yl)carbonyl]phenyl}-2-(2-pyrrolidin-1-ylethyl)-3,4-dihydroisoquinolin-1(2H)-one;
6-(4-{[(2S)-2-(methoxymethyl)pyrrolidin-1-yl]carbonyl}phenyl)-2-(2-pyrrolidin-1-ylethyl)-3,4-dihydroisoquinolin-1(2H)-one;
4-[1-oxo-2-(2-pyrrolidin-1-ylethyl)-1,2,3,4-tetrahydroisoquinolin-6-yl]-N-propylbenzamide;
4-[1-oxo-2-(2-pyrrolidin-1-ylethyl)-1,2,3,4-tetrahydroisoquinolin-6-yl]-N-1,3-thiazol-2-ylbenzamide;
6-[4-fluoro-3-(pyrrolidin-1-ylcarbonyl)phenyl]-2-(2-pyrrolidin-1-ylethyl)-3,4-dihydroisoquinolin-1-(2H)-one;
2-fluoro-N,N-dimethyl-5-[1-oxo-2-(2-pyrrolidin-1-ylethyl)-1,2,3,4-tetrahydroisoquinolin-6-yl]benzamide;
3-fluoro-N,N-dimethyl-4-[1-oxo-2-(2-pyrrolidin-1-ylethyl)-1,2,3,4-tetrahydroisoquinolin-6-yl]benzamide;
6-[2-fluoro-4-(pyrrolidin-1-ylcarbonyl)phenyl]-2-(2-pyrrolidin-1-ylethyl)-3,4-dihydroisoquinolin-1(2H)-one;
6-[3-fluoro-4-(pyrrolidin-1-ylcarbonyl)phenyl]-2-(2-pyrrolidin-1-ylethyl)-3,4-dihydroisoquinolin-1(2H)-one;
6-[3-chloro-4-(pyrrolidin-1-ylcarbonyl)phenyl]-2-(2-pyrrolidin-1-ylethyl)-3,4-dihydroisoquinolin-1(2H)-one;
2-{2-[(2R)-2-methylpyrrolidin-1-yl]ethyl}-6-[4-(pyrrolidin-1-ylcarbonyl)phenoxy]-3,4-dihydroisoquinolin-1(2H)-one;
N-ethyl-4-[2-(2-pyrrolidin-1-ylethyl)-1,2,3,4-tetrahydroisoquinolin-6-yl]benzamide;
N-methyl-4-[2-(2-pyrrolidin-1-ylethyl)-1,2,3,4-tetrahydroisoquinolin-6-yl]benzamide;
6-[4-(pyrrolidin-1-ylcarbonyl)phenyl]-2-(2-pyrrolidin-1-ylethyl)-1,2,3,4-tetrahydroisoquinoline;
N,N-dimethyl-4-[2-(2-pyrrolidin-1-ylethyl)-1,2,3,4-tetrahydroisoquinolin-6-yl]benzamide;
6-[4-(piperidin-1-ylcarbonyl)phenyl]-2-(2-pyrrolidin-1-ylethyl)-1,2,3,4-tetrahydroisoquinoline;
6-[4-(morpholin-4-ylcarbonyl)phenyl]-2-(2-pyrrolidin-1-ylethyl)-1,2,3,4-tetrahydroisoquinoline;
4-[2-(2-pyrrolidin-1-ylethyl)-1,2,3,4-tetrahydroisoquinolin-6-yl]benzamide;

N-methyl-4-[1-oxo-2-(3-pyrrolidin-1-ylpropyl)-1,2,3,4-tetrahydroisoquinolin-6-yl]benzamide;
6-(4-{[(2S)-2-methylpyrrolidin-1-yl]carbonyl}phenyl)-2-(2-pyrrolidin-1-ylethyl)-1,2,3,4-tetrahydroisoquinoline;
6-(1H-pyrazol-1-yl)-2-(2-pyrrolidin-1-ylethyl)-3,4-dihydroisoquinolin-1(2H)-one;
6-(1H-indazol-1-yl)-2-(2-pyrrolidin-1-ylethyl)-3,4-dihydroisoquinolin-1(2H)-one;
2-{2-[(2R)-2-methylpyrrolidin-1-yl]ethyl}-3,3′,4,4′-tetrahydro-6,6′-biisoquinoline-1,1′(2H,2′H)-dione;
6-(azepan-1-ylcarbonyl)-2-{2-[(2R)-2-methylpyrrolidin-1-yl]ethyl}-3,4-dihydroisoquinolin-1(2H)-one;
N-cyclobutyl-2-{2-[(2R)-2-methylpyrrolidin-1-yl]ethyl}-1-oxo-1,2,3,4-tetrahydroisoquinoline-6-carboxamide;
2-{2-[(2R)-2-methylpyrrolidin-1-yl]ethyl}-6-(piperidin-1-ylcarbonyl)-3,4-dihydroisoquinolin-1(2H)-one;
N-cyclohexyl-2-{2-[(2R)-2-methylpyrrolidin-1-yl]ethyl}-1-oxo-1,2,3,4-tetrahydroisoquinoline-6-carboxamide;
N-(2,3-dihydro-1H-inden-2-yl)-2-{2-[(2R)-2-methylpyrrolidin-1-yl]ethyl}-1-oxo-1,2,3,4-tetrahydroisoquinoline-6-carboxamide;
2-{2-[(2R)-2-methylpyrrolidin-1-yl]ethyl}-1-oxo-N-pyridin-4-yl-1,2,3,4-tetrahydroisoquinoline-6-carboxamide;

N-cyclopentyl-2-{2-[(2R)-2-methylpyrrolidin-1-yl]ethyl}-1-oxo-1,2,3,4-tetrahydroisoquinoline-6-carboxamide;
6-(3,4-dihydroisoquinolin-2(1H)-ylcarbonyl)-2-{2-[(2R)-2-methylpyrrolidin-1-yl]ethyl}-3,4-dihydroisoquinolin-1(2H)-one;
2-{2-[(2R)-2-methylpyrrolidin-1-yl]ethyl}-6-(pyrrolidin-1-ylcarbonyl)-3,4-dihydroisoquinolin-1(2H)-one;
6-(1,3-dihydro-2H-isoindol-2-ylcarbonyl)-2-{2-[(2R)-2-methylpyrrolidin-1-yl]ethyl}-3,4-dihydroisoquinolin-1(2H)-one;
6-(4-fluorophenyl)-2-{2-[(2R)-2-methylpyrrolidin-1-yl]ethyl}-1,2,3,4-tetrahydroisoquinoline;
2-(2-pyrrolidin-1-ylethyl)-6-[4-(trifluoromethoxy)phenyl]-1,2,3,4-tetrahydroisoquinoline;
6-(3-fluorophenyl)-2-(2-pyrrolidin-1-ylethyl)-1,2,3,4-tetrahydroisoquinoline;
6-(1,3-benzodioxol-5-yl)-2-(2-pyrrolidin-1-ylethyl)-1,2,3,4-tetrahydroisoquinoline;
6-(4-fluorophenyl)-2-(2-piperidin-1-ylethyl)-1,2,3,4-tetrahydroisoquinoline;
2-(2-azepan-1-ylethyl)-6-(4-fluorophenyl)-1,2,3,4-tetrahydroisoquinoline;
3-fluoro-N-methyl-4-[1-oxo-2-(2-pyrrolidin-1-ylethyl)-1,2,3,4-tetrahydroisoquinolin-6-yl]benzaimde;
N-ethyl-3-fluoro-4-[1-oxo-2-(2-pyrrolidin-1-ylethyl)-1,2,3,4-tetrahydroisoquinolin-6-yl]benzamide;
6-(1H-benzimidazol-1-yl)-2-{2-[(2R)-2-methylpyrrolidin-1-yl]ethyl}-3,4-dihydroisoquinolin-1(2H)-one;
2-{2-[(2R)-2-methylpyrrolidin-1-yl]ethyl}-5-[4-(pyrrolidin-1-ylcarbonyl)phenyl]-isoindolin-1-one;
N-methyl-4-(2-{2-[(2R)-2-methylpyrrolidin-1-yl]ethyl}-1-oxo-2,3-dihydro-1H-isoindol-5-yl)benzamide;
6-[4-(methylsulfonyl)phenyl]-2-(2-pyrrolidin-1-ylethyl)-3,4-dihydroisoquinolin-1(2H)-one;
2-{2-[(2R)-2-methylpyrrolidin-1-yl]ethyl}-6-piperidin-1-yl-3,4-dihydroisoquinolin-1(2H)-one;
6-(piperidin-1-yl)-2-(2-(pyrrolidin-1-yl)ethyl)-3,4-dihydroisoquinolin-1(2H)-one;
6-(piperidin-1-yl)-2-(2-(piperidin-1-yl)ethyl)-3,4-dihydroisoquinolin-1(2H)-one;
2-(2-(azepan-1-yl)ethyl)-6-(piperidin-1-yl)-3,4-dihydroisoquinolin-1(2H)-one;
(R)-2-(2-(2-methylpyrrolidin-1-yl)ethyl)-6-(pyrrolidin-1-yl)-3,4-dihydroisoquinolin-1(2H)-one;
(R)-6-(azepan-1-yl)-2-(2-(2-methylpyrrolidin-1-yl)ethyl)-3,4-dihydroisoquinolin-1(2H)-one;
(R)-2-methyl-2′-(2-(2-methylpyrrolidin-1-yl)ethyl)-3,3′,4,4′-tetrahydro-6,6′-biiso-quinoline-1,1′(2H,2′H)-dione;
2-methyl-2′-(2-(pyrrolidin-1-yl)ethyl)-3,3′,4,4′-tetrahydro-6,6′-biisoquinoline-1,1′(2H,2′H)-dione;
2-(3-(pyrrolidin-1-yl)propyl)-6-(4-(pyrrolidine-1-carbonyl)phenyl)-3,4-dihydroiso-quinolin-1(2H)-one;
6-(isoindoline-2-carbonyl)-2-(2-(pyrrolidin-1-yl)ethyl)-3,4-dihydroisoquinolin-1(2H)-one;
6-(piperidine-1-carbonyl)-2-(2-(pyrrolidin-1-yl)ethyl)-3,4-dihydroisoquinolin-1(2H)-one;
(R)-N,N-dimethyl-4-(2-(2-(2-methylpyrrolidin-1-yl)ethyl)-1-oxo-1,2,3,4-tetrahydro-isoquinolin-6-yl)benzamide;
(R)-6-(4-(azetidine-1-carbonyl)phenyl)-2-(2-(2-methylpyrrolidin-1-yl)ethyl)-3,4-dihydroisoquinolin-1(2H)-one;
(R)-2-(2-(2-methylpyrrolidin-1-yl)ethyl)-6-(4-(piperidine-1-carbonyl)phenyl)-3,4-dihydroisoquinolin-1(2H)-one;
(R)-2-(2-(2-methylpyrrolidin-1-yl)ethyl)-6-(4-(morpholine-4-carbonyl)phenyl)-3,4-dihydroisoquinolin-1(2H)-one;
(R)-N-(2-methoxyethyl)-4-(2-(2-(2-methylpyrrolidin-1-yl)ethyl)-1-oxo-1,2,3,4-tetrahydroisoquinolin-6-yl)benzamide;
(R)-N-(2-isopropoxyethyl)-4-(2-(2-(2-methylpyrrolidin-1-yl)ethyl)-1-oxo-1,2,3,4-tetrahydroisoquinolin-6-yl)benzamide;
N-((S)-1-methoxypropan-2-yl)-4-(2-(2-((R)-2-methylpyrrolidin-1-yl)ethyl)-1-oxo-1,2,3,4-tetrahydroisoquinolin-6-yl)benzamide;
(R)-N-(2-fluoroethyl)-4-(2-(2-(2-methylpyrrolidin-1-yl)ethyl)-1-oxo-1,2,3,4-tetrahydro-isoquinolin -6-yl)benzamide;
6-(4-((S)-2-(methoxymethyl)pyrrolidine-1-carbonyl)phenyl)-2-(2-((R)-2-methyl-pyrrolidin-1-yl)ethyl)-3,4-dihydroisoquinolin-1(2H)-one;
(R)-N-ethyl-N-methyl-4-(2-(2-(2-methylpyrrolidin-1-yl)ethyl)-1-oxo-1,2,3,4-tetrahydroisoquinolin-6-yl)benzamide;
   or a stereoisomer thereof or a pharmaceutically acceptable salt thereof.

Advantageously, the present invention provides a process to prepare compounds of formula I which comprises reacting an aldehyde of formula II with a pyrrolidine of formula III in the presence of NaBH₃CN optionally in the presence of an acid optionally in the presence of a solvent. The reaction is shown in scheme I.

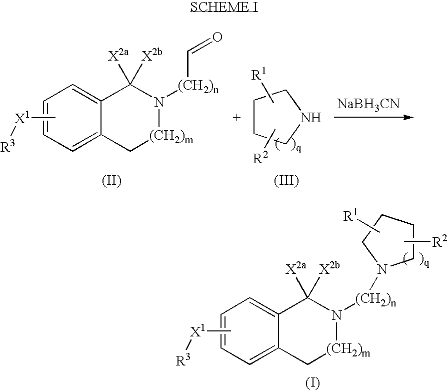

Acids suitable for use in the method of invention include carboxylic acids such as acetic acid, propanoic acid, or the like, preferably acetic acid. Solvents suitable for use in the method of the invention include alcohols such as methanol.

Compounds of formula II may be readily prepared by reacting a compound of formula IV with sodium azide and methylsulfonic acid to give the lactam of formula V; reacting said formula V lactam with an alkenylbromide of formula VI in the presence of a base such as NaH to give the compound of formula VII; and oxidizing the formula VII compound with an oxidizing agent such as, osmium tetraoxide and sodium periodate to provide the desired aldehyde of formula II. The reaction is shown in scheme II.

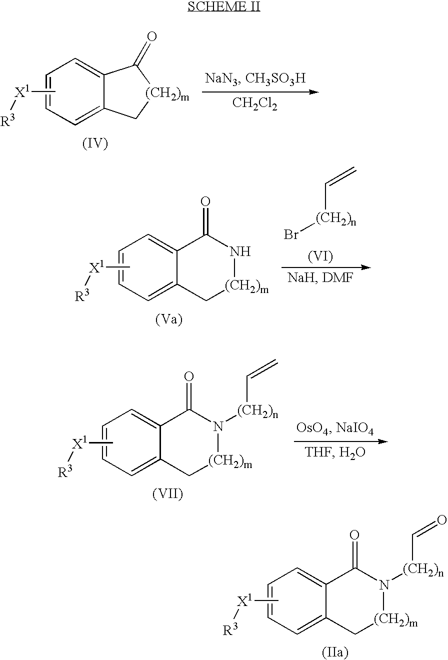

Alternatively, compounds of formula II may be prepared by reacting the lactam of formula V with a bromoalkyl-1,3-dioxane of formula VIII in the presence of a base such as NaH to give the compound of formula IX and hydrolyzing said formula IX compound using acidic conditions to give the desired aldehyde of formula II. The reaction is shown in flow diagram III.

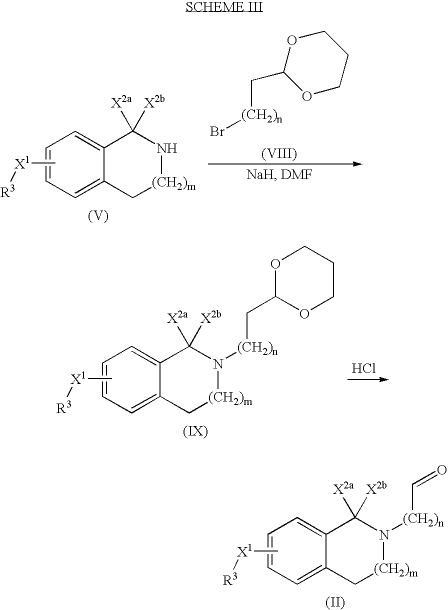

Compounds of formula II wherein X¹ is (CH₂)ₚ and R³ is an optionally substituted aryl or heteroaryl group (IIa) may be conveniently prepared by reacting a lactam of formula X with an alkenylbromide of formula VI as shown in scheme II to give the compound of formula XI and coupling the formula XI compound with an aryl or heteroaryl boronic acid of formula XII in the presence of a palladium catalyst such as dichlorobis(tri-o-tolylphosphine)palladium and a base such as K₂CO₃ to give the compound of formula XIII; and oxidizing the formula XIII compound to give the desired compound of formula IIa. The reaction is shown in scheme IV wherein Hal represents Cl, Br, I or triflate and R³ is an optionally substituted aryl or heteroaryl group.

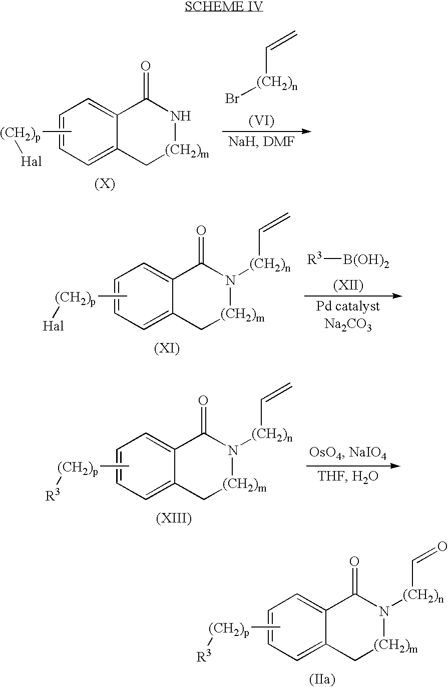

Alternatively, compounds of formula XIII wherein m and p are 0 (XIIIa) may be prepared by reacting a 2-methylbenzoic acid of formula XIV with trimethylsilyldiazomethane (TMSCHN₂) to give the corresponding methyl ester; reacting said ester with N-bromosuccinimide (NBS) and benzoylperoxide to give the compound of formula XV; and reacting the formula XV compound with an alkenyl-amine of formula XVI to give the desired compound of formula XIIIa. The reaction is shown in scheme V.

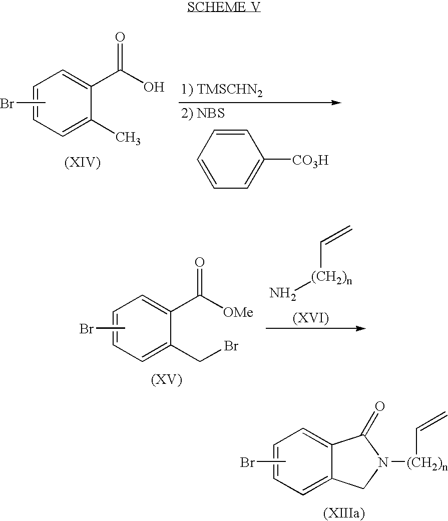

Compounds of formula II wherein X is 0 (IIb) may be readily prepared by reacting a lactam of formula XVII with boron tribromide to give the corresponding hydroxy compound of formula XVIII; reacting said formula XVIII compound with an aryl or heteroaryl halide of formula XIX in the presence of a base such as K₂CO₃ to give the compound of formula XX; and reacting the formula XX compound with an alkenylbromide of formula VI followed by oxidation with OsO₄ and NalO₄, as shown in scheme II, to give the desired compound of formula IIb. The reaction is shown in scheme VI wherein Hal is F, Cl, Br or I.

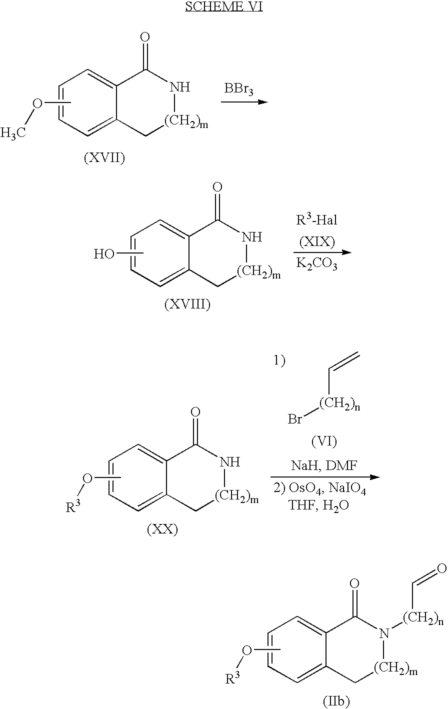

The compounds of formula IIa and IIb may be converted to the corresponding compounds of formula I as shown in scheme I.

Compounds of formula Ib may also be prepared by first building the desired cycloamin-1-ylalkyl side chain on a suitable lactam substrate and then forming the desired X—R³ substitution, for example compounds of formula I wherein X is CO and R³ is NR⁶R⁷ (Ib) may be prepared by reacting the lactam of formula X wherein p is 0 (Xa) with an alkenylbromide of formula VI, followed by oxidation and reductive amination with formula III and NaBH₃CN, as shown in schemes I and II, to give the compound of formula XXI; and where Hal is Cl, Br or I, or a leaving group such as triflate, reacting the formula XXI compound with Cul and Nal to give the corresponding iodide compound; and reacting said iodide compound with an amine, HNR⁶R⁷, carbon monoxide, a palladium source such as dichlorobis(tri-phenylphosphine)palladium (II) and a base such as triethylamine to give the desired compound of formula Ib. The reaction is shown in scheme VII, wherein Hal is Cl, Br or I, or a leaving group such as triflate.

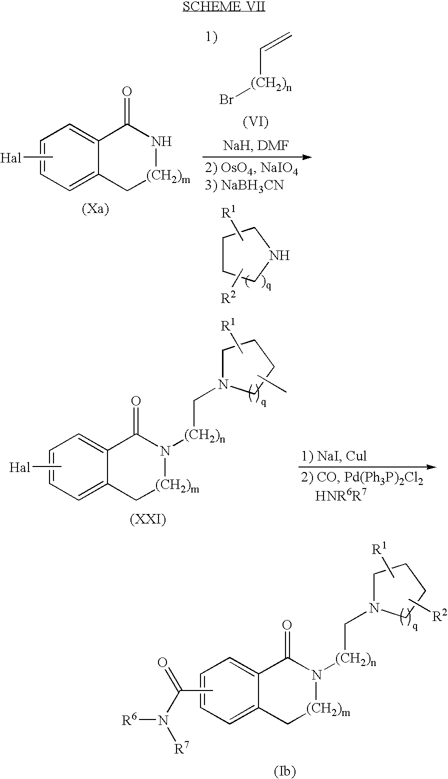

Compounds of formula (Ic) may also be prepared from compounds of formula (Ia) by reduction in presence of lithium aluminum hydride in tetrahydrofuran. The reaction is shown in scheme VIII.

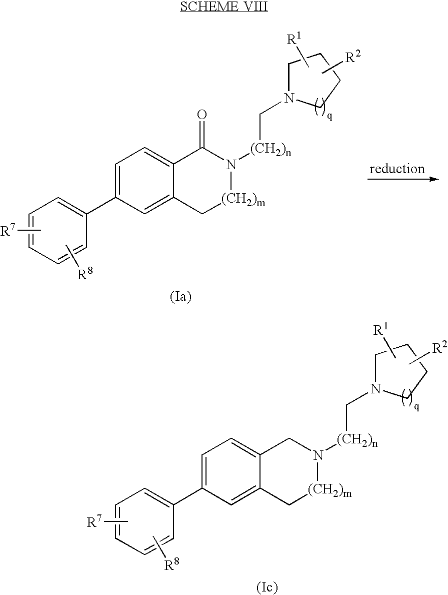

Compounds of formula (Ic) may also be prepared from compounds of formula (XXI) by reduction in presence of lithium aluminum hydride in tetrahydrofuran; reacting said formula XXII with boron tribromide to give the compound of formula XXIII; reacting said formula XXIII compound with triflate reagent, such as Tf₂NPh and a base such as triethyl amine, to generate the compound of formula XXIV, reacting said formula XXIV compound with a boronic acid of formula XII in the presences of a palladium catalysts such as dichlorobis(tri-o-tolyphosphine)-palladium (II) and a base such as K₂CO₃ to give the compound of formula Ic; The reaction is shown in scheme IX.

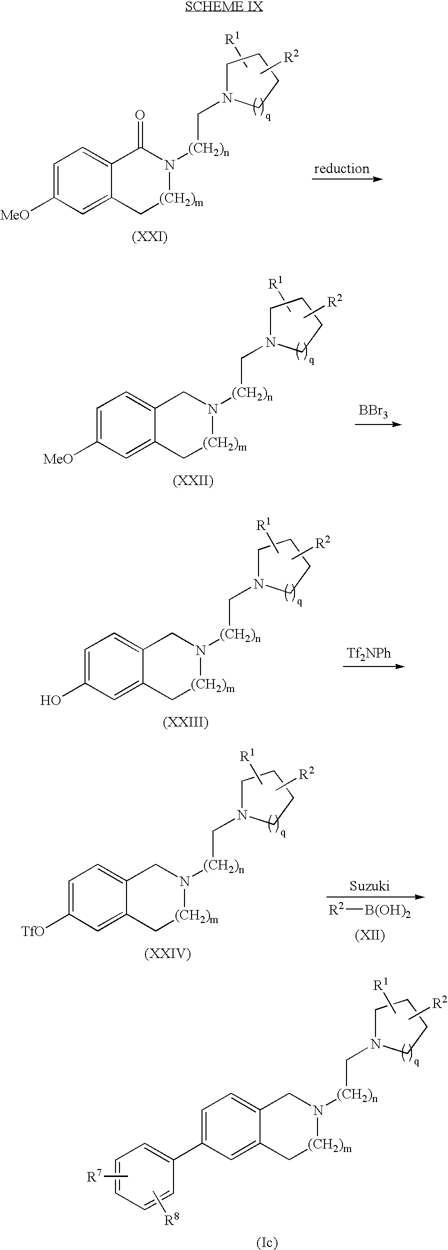

Compounds of formula Id wherein R³ is NR⁶R⁷ and Hal is fluorine may be prepared by reacting formula XXV with an amine of formula HNR⁶R⁷ in the presences of a base, such as K₂CO3 to give compounds of formula Id. The reaction is shown in reaction scheme X.

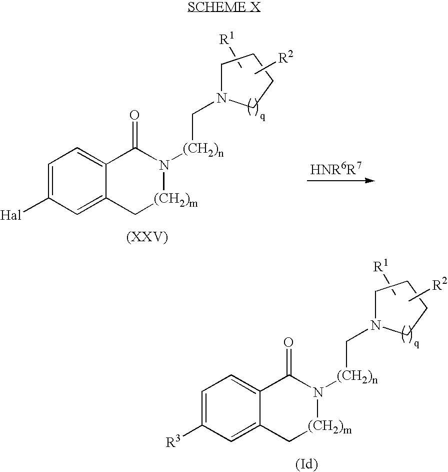

Alternatively, compounds of formula Id wherein R³ is NR⁶R⁷ and Hal is fluorine may be prepared by reacting formula XXVI with an amine of formula HNR⁶R⁷ in the presences of a base, such as K₂CO₃ to give compounds of formula XXVII; and reacting the formula XXVII compound with an alkenylbromide followed by oxidation with OSO₄/NalO₄ and reductive amination, as shown in schemes II and I, to give the desired compound of formula Id. The reaction is shown in scheme XI.

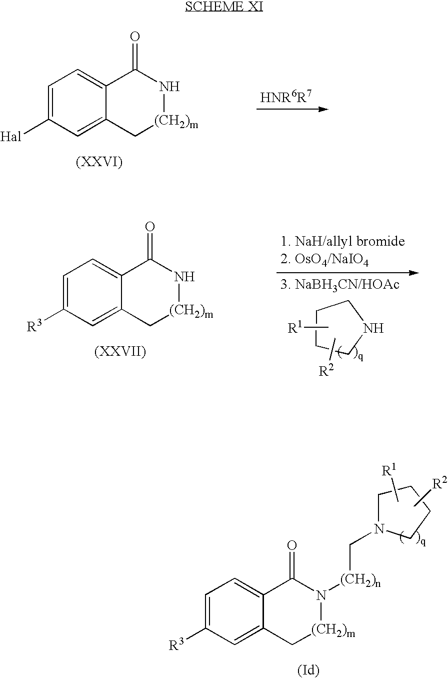

Advantageously, the formula I compounds of the invention are useful for the treatment of CNS disorders related to or affected by the Histamine-3 receptor including cognitive disorders, for example Alzheimer's disease, mild cognitive impairment, attention deficit hyperactivity disorder, schizophrenia, memory loss, obesity, sleep disorders, eating disorders, neuropathic pain or the like. Accordingly, the present invention provides a method for the treatment of a disorder of the central nervous system related to or affected by the Histamine-3 receptor in a patient in need thereof which comprises providing said patient a therapeutically effective amount of a compound of formula I as described hereinabove. The compounds may be provided by oral or parenteral administration or in any common manner known to be an effective administration of a therapeutic agent to a patient in need thereof.

The term “providing” as used herein with respect to providing a compound or substance embraced by the invention, designates either directly administering such a compound or substance, or administering a prodrug, derivative or analog which forms an equivalent amount of the compound or substance within the body.

The inventive method includes: a method for the treatment of schizophrenia; a method for the treatment of a disease associated with a deficit in memory, cognition or learning or a cognitive disorder such as Alzheimer's disease or attention deficit hyperactivity disorder; a method for the treatment of a mild cognitive disorder, a method for the treatment of a developmental disorder such as schizophrenia; a method for the treatment of a sleep disorder, a method for the treatment of an eating disorder, a method for the treatment of neuropathic pain or any other CNS disease or disorder associated with or related to the H₃ receptor.

In one embodiment, the present invention provides a method for treating attention deficit hyperactivity disorders (ADHD, also known as Attention Deficit Disorder or ADD) in both children and adults. Accordingly, in this embodiment, the present invention provides a method for treating attention deficit disorders in a pediatric patient.

The present invention therefore provides a method for the treatment of each of the conditions listed above in a patient, preferably in a human, said method comprises providing said patient a therapeutically effective amount of a compound of formula I as described hereinabove. The compounds may be provided by oral or parenteral administration or in any common manner known to be an effective administration of a therapeutic agent to a patient in need thereof.

The therapeutically effective amount provided in the treatment of a specific CNS disorder may vary according to the specific condition(s) being treated, the size, age and response pattern of the patient, the severity of the disorder, the judgment of the attending physician and the like. In general, effective amounts for daily oral administration may be about 0.01 to 1,000 mg/kg, preferably about 0.5 to 500 mg/kg and effective amounts for parenteral administration may be about 0.1 to 100 mg/kg, preferably about 0.5 to 50 mg/kg.

In actual practice, the compounds of the invention are provided by administering the compound or a precursor thereof in a solid or liquid form, either neat or in combination with one or more conventional pharmaceutical carriers or excipients. Accordingly, the present invention provides a pharmaceutical composition which comprises a pharmaceutically acceptable carrier and an effective amount of a compound of formula I as described hereinabove.

In one embodiment, the invention relates to compositions comprising at least one compound of formula I, or a pharmaceutically acceptable salt thereof, and one or more pharmaceutically acceptable carriers, excipients, or diluents. Such compositions include pharmaceutical compositions for treating or controlling disease states or conditions of the central nervous system. In certain embodiments, the compositions comprise mixtures of one or more compounds of formula I.

In certain embodiments, the invention relates to compositions comprising at least one compound of formula I, or a pharmaceutically acceptable salt thereof, and one or more pharmaceutically acceptable carriers, excipients, or diluents. Such compositions are prepared in accordance with acceptable pharmaceutical procedures. Pharmaceutically acceptable carriers are those carriers that are compatible with the other ingredients in the formulation and are biologically acceptable.

The compounds of formula I may be administered orally or parenterally, neat, or in combination with conventional pharmaceutical carriers. Applicable solid carriers can include one or more substances that can also act as flavoring agents, lubricants, solubilizers, suspending agents, fillers, glidants, compression aids, binders, tablet-disintegrating agents, or encapsulating materials. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

In certain embodiments, a compound of formula I is provided in a disintegrating tablet formulation suitable for pediatric administration.

Liquid carriers can be used in preparing solutions, suspensions, emulsions, syrups and elixirs. The active ingredient can be dissolved or suspended in a pharmaceutically acceptable liquid carrier such as water, an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

In certain embodiments, a liquid pharmaceutical composition is provided wherein said composition is suitable for pediatric administration. In other embodiments, the liquid composition is a syrup or suspension.

Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form.

The compounds of formula I may be administered rectally or vaginally in the form of a conventional suppository. For administration by intranasal or intrabronchial inhalation or insufflation, the compounds of formula I can be formulated into an aqueous or partially aqueous solution, which can then be utilized in the form of an aerosol. The compounds of formula I can also be administered transdermally through the use of a transdermal patch containing the active compound and a carrier that is inert to the active compound, is non-toxic to the skin, and allows delivery of the agent for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable. A variety of occlusive devices can be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form.

The therapeutically effective amount of a compound of formula I provided to a patient will vary depending upon what is being administered, the purpose of the administration, such as prophylaxis or therapy, the state of the patient, the manner of administration, or the like. In therapeutic applications, compounds of formula I are provided to a patient suffering from a condition in an amount sufficient to treat or at least partially treat the symptoms of the condition and its complications. An amount adequate to accomplish this is a “therapeutically effective amount” as described previously herein. The dosage to be used in the treatment of a specific case must be subjectively determined by the attending physician. The variables involved include the specific condition and the size, age, and response pattern of the patient. Generally, a starting dose is about 5 mg per day with gradual increase in the daily dose to about 150 mg per day, to provide the desired dosage level in the patient.

In certain embodiments, the present invention is directed to prodrugs of compounds of formula I. The term “prodrug,” as used herein, means a compound that is convertible in vivo by metabolic means (e.g. by hydrolysis) to a compound of formula I. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975).

For a more clear understanding, and in order to illustrate the invention more clearly, specific examples thereof are set forth hereinbelow. The following examples are merely illustrative and are not to be understood as limiting the scope and underlying principles of the invention in any way. The terms DMF and THF designate dimethyl formamide and tetrehydrofuran, respectively. The terms HPLC and NMR designate high performance liquid chromatography and proton nuclear magnetic resonance, respectively. The term MS designates mass spectroscopy with (+) referring to the positive mode which generally gives a M+1 (or M+H) absorption where M=the molecular mass. All compounds are analyzed at least by MS and NMR. Unless otherwise noted, all parts are parts by weight.

### EXAMPLES

### Example 1

### Preparation of bromo-substituted 3,4-dihydroisoquinolin-1(2H)-ones

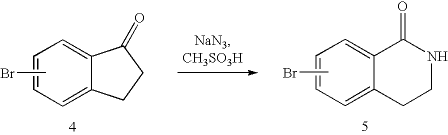

### 6-Bromo-3,4-dihydroisoquinolin-1(2H)-one (1a)

A solution of 5-bromo-1-indanone (1.08 g, 5.1 mmol) in (2:1) methylene chloride: methanesulfonic acid (45 mL) at 0° C. was treated slowly with sodium azide (0.5 g, 7.7 mmol), allowed to warm to room temperature, stirred overnight and partitioned between methylene chloride and aqueous sodium hydroxide (50 mL, 1.0 N). The aqueous layer was extracted with methylene chloride. The combined organic layers were washed sequentially with water and brine, dried over Na₂SO₄ and concentrated in vacuo. The residue was purified by ISCO CombiFlash® chromatography (silica, 10-100% ethyl acetate in hexanes) to afford the title compound as a white solid, 0.45 g (39%), mp 137-139° C.; MS (ES) m/z [M+H]⁺ 226.0.

### 7-Bromo-3,4-dihydroisoquinolin-1(2H)-one (1b)

According the procedure described in 1a and employing 6-bromo-1-indanone (4.0 g, 19 mmol), 2.64 g (47%) of 7-bromo-3,4-dihydroisoquinolin-1(2H)-one was obtained as a white solid. mp 100-102° C. MS (ES) m/z 225.9 [M+H]⁺.

### 5-Bromo-3,4-dihydroisoquinolin-1(2H)-one (1c)

According the procedure described in la and employing 4-bromo-1-indanone (2.0 g, 9.5 mmol), 1.60 g (75%) of 5-bromo-3,4-dihydroisoquinolin-1(2H)-one was obtained as a white solid. MS (ES) m/z 226.0 [M+H]⁺.

### Example 2

### Preparation of 6-methoxy-3,4-dihydroisoquinolin-1(2H)-one

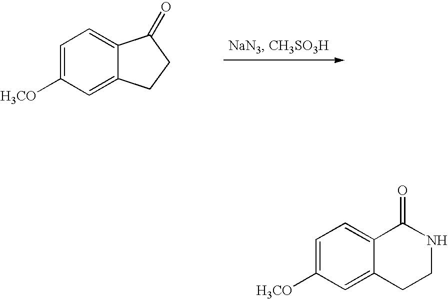

Using essentially the same procedure described in Example 1 and employing 5-methoxy-1-indanone (4.98 g, 31 mmol), the title compound 4.5 g (82%) was obtained as a white solid, mp 98-100° C.; MS (ES) m/z 178.0 [M+H]⁺.

### Example 3

### Preparation of bromo substituted 2-allyl-3,4-dihydroisoquinolin-1(2H)-ones

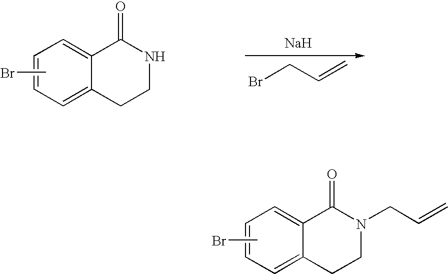

### 2-Allyl-6-bromo-3,4-dihydroisoquinolin-1(2H)-one (3a)

A suspension of sodium hydride (60% dispersion in mineral oil, 0.17 g, 4.4 mmol) in N,N-dimethylformamide at 0° C., under nitrogen, was treated dropwise over 15 min with a solution of 6-bromo-3,4-dihydroisoquinolin-1(2)-one (0.5 g, 2.2 mmol) in N,N-dimethylformamide, stirred at 0° C. for an additional 20 min, treated with allyl bromide (0.29 mL, 3.3 mmol) at 0° C., allowed to warm to room temperature, stirred overnight and was partitioned between water and methylene chloride. The aqueous layer was extracted with methylene chloride. The combined extracts and the organic layer were washed with brine, dried over Na₂SO₄ and concentrated in vacuo. The residue was purified by ISCO CombiFlash® chromatography (silica, 10-50% ethyl acetate in hexanes) to afford the title compound as a light yellow oil, 0.55 g (93%), MS (ES) m/z 266.0 [M+H]⁺.

### 2-Allyl-7-bromo-3,4-dihydroisoquinolin-1(2H)-one (3b)

According to the procedure described for 3a and starting from 7-bromo-3,4-dihydroisoquinolin-1(2H)-one (2.84 g, 12 mmol), 2.2 g (63%) of 2-allyl-7-bromo-3,4-dihydroisoquinolin-1(2H)-one was obtained as a light yellow oil. MS (ES) m/z 266.0 [M+H]⁺.

### 2-Allyl-5-bromo-3,4-dihydroisoquinolin-1(2H)-one (3c)

According to the procedure described for 3a and starting from 5-bromo-3,4-dihydroisoquinolin-1(2H)-one (1.6 g, 7.0 mmol), 0.97 (51%) of 2-allyl-5-bromo-3,4-dihydroisoquinolin-1(2H)-one was obtained as a light yellow oil. MS (ES) m/z 266.0 [M+H]⁺.

### Example 4

### Preparation of 4-(2-allyl-1-oxo-1,2,3,4-tetrahydroisoquinolin-6-yl)benzonitrile

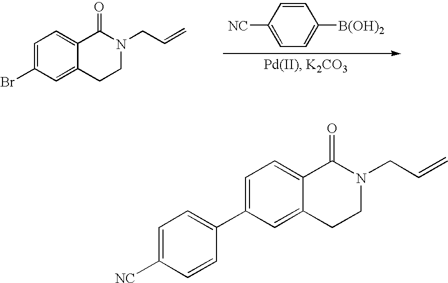

A solution of 2-allyl-6-bromo-3,4-dihydroisoquinolin-1(2H)-one (1.22 g, 4.6 mmol) and 4-cyanobenzene boronic acid (2.7 g, 18 mmol) in dioxane at 90° C. was treated with dichlorobis(tri-o-tolyphosphine)palladium (II) (0.18 g, 0.23 mmol), potassium carbonate (1.6 g, 11.5 mmol) and water, heated at 90° C. for 0.5 h, cooled to room temperature and filtered through a pad of celite. The filtrate was partitioned between aqueous sodium hydroxide and dichloromethane. The aqueous phase was separated and extracted with dichloromethane. The combined organic phases were concentrated in vacuo. The residue was purified by ISCO CombiFlash® chromatography (silica, 10-100% ethyl acetate in hexanes) to afford the title compound as a colorless oil, 1.04 g (79%), MS (ES) m/z 289.1 [M+H]⁺.

### Example 5

### Preparation of 2-allyl-6-(4-fluorophenyl)-3,4-dihydroisoquinolin-1(2H)-one

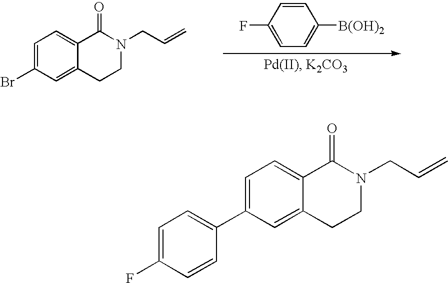

Using essentially the same procedure described in Example 4 and employing 2-allyl-6-bromo-3,4-dihydroisoquinolin-1(2H)-one (1.23 g, 4.6 mmol) and 4-fluorobenzene boronic acid (2.6 g, 18 mmol), the title compound was obtained as a colorless oil, 1.06 g (81%), MS (ES) m/z 282.1 [M+H]⁺.

### Example 6

### Preparation of 2-(bromo substituted-1-oxo-3,4-dihydroisoquinolin-2(1-yl)acetaldehydes

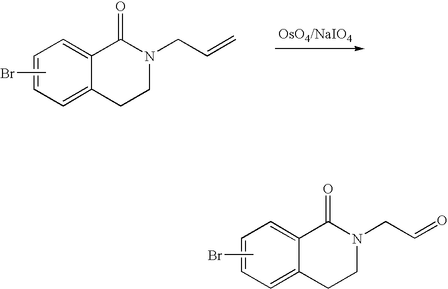

### 2-(6-Bromo-1-oxo-3,4-dihydroisoquinolin-2(1H)-yl)acetaldehyde (6a)

A solution of 2-allyl-6-bromo-3,4-dihydroisoquinolin-1(2H)-one (3.11 g, 12 mmol) in tetrahydrofuran and water at 0° C. was treated with sodium periodate (7.5 g, 36 mmol), allowed to stir at 0° C. for 10 min, treated with osmium (VIII) tetraoxide (4 wt. % solution in water, 1.5 mL) at 0° C., stirred at 0° C. for 8 h, poured into water and extracted with methylene chloride. The combined extracts were washed with brine, dried over Na₂SO₄ and concentrated in vacuo. The residue was purified by ISCO CombiFlash® chromatography (silica, 40-100% ethyl acetate in hexanes) to afford the title compound as a colorless oil, 2.74 g (87%), HRMS (ES) m/z 267.9966 [M+H]⁺.

### 2-(7-Bromo-1-oxo-3,4-dihydroisoquinolin-2(1H)-yl)acetaldehyde (6b)

According to the procedure described for 6a and starting from 2-allyl-7-bromo-3,4-dihydroisoquinolin-1(2H)-one (2.2 g, 7.8 mmol, 1.17 g (56%) of the title product was obtained as a white solid. mp 95-96° C. MS (ES) m/z 266.0 [M+H]⁺.

### 2-(5-Bromo-1-oxo-3,4-dihydroisoquinolin-2(1H)-yl)acetaldehyde (6c)

According to the procedure described for 6a and starting from 2-allyl-5-bromo-3,4-dihydroisoquinolin-1(2H)-one (0.97 g, 3.4 mmol), 0.74 g (80%) of 2-(5-bromo-1-oxo-3,4-dihydroisoquinolin-2(1H)-yl)acetaldehyde was obtained as a light yellow oil. MS (ES) m/z 268.0 [M+H]⁺.

### Example 7

### Preparation of 6-bromo-2-[2-(pyrrolidin-1-yl)ethyl]-3,4-dihydroisoquinolin-1(2H)-one

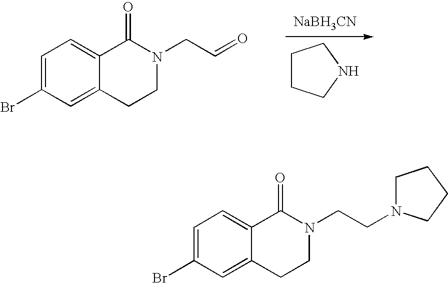

A stirred solution of 2-(6-bromo-1-oxo-3,4-dihydroisoquinolin-2(1H)-yl)acetaldehyde (2.7 g, 11 mmol) and pyrrolidine (1.24 mL, 16.5 mmol) in methanol (40 mL) was treated with NaBH₃CN (0.95 g, 16.5 mmol) and acetic acid (1.44 mL, 27.5 mmol) at room temperature, stirred overnight, diluted with CH₂Cl₂ and washed with saturated NaHCO₃. The aqueous layer was extracted with methylene chloride. The combined organic layers were washed with brine, dried over Na₂SO₄ and concentrated in vacuo. The residue was purified by ISCO CombiFlash® chromatography (silica, 0-10% methanol in methylene with 0.5% ammonium hydroxide) to afford the title compound as a colorless oil, 2.40 g (74%), MS (ES) m/z 323.1 [M+H]⁺.

### Example 8

### Preparation of (R)-bromo substituted-2-[2-(2-methylpyrrolidin-1-yl)ethyl]-3,4-dihydroisoquinolin-1(2H)-ones

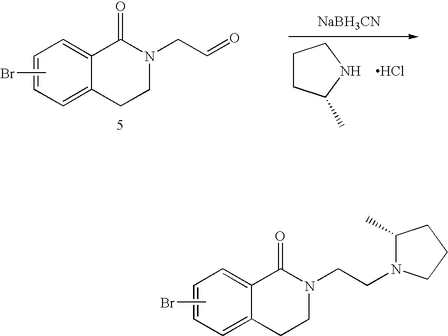

### (R)-6-Bromo-2-[2-(2-methylpyrrolidin-1-yl)ethyl]-3,4-dihydroisoquinolin-1(2H)-one (8a)

Using essentially the same procedure described in Example 7 and employing 2-(6-bromo-1-oxo-3,4-dihydroisoquinolin-2(1H)-yl)acetaldehyde (2.60 g, 9.7 mmol), (R)-2-methylpyrrolidine hydrochloride (1.4 g, 11.6 mmol) and diisopropylethylamine (2.0 mL, 11.6 mmol), the title compound was obtained as a colorless oil, 2.70 g (83%), [α]_{D}²⁵=−59.8° (c=1.00 in methanol); MS (ES) m/z 337.1 [M+H]⁺.

### (R)-7-Bromo-2-[2-(2-methylpyrrolidin-1-yl)ethyl]-3,4-dihydroisoquinolin-1(2H)-one (8b)

Using essentially the same procedure described in Example 7, starting from 2-(7-bromo-1-oxo-3,4-dihydroisoquinolin-2(1H)-yl)acetaldehyde (1.17 g, 4.4 mmol), (R)-2-methylpyrrolidine hydrochloride (0.63 g, 5.2 mmol) and diisopropylethylamine (0.91 mL, 5.2 mmol), 0.59 g (40%) of the title product was obtained as a colorless oil. [α]=−48° (1% solutionin Methanol), MS (ES) m/z 337.1 [M+H]⁺.

### (R)-5-Bromo-2-[2-(2-methylpyrrolidin-1-yl)ethyl]-3,4-dihydroisoquinolin-1(2H)-one (8c)

Using essentially the same procedure described in Example 7, starting from 2-(5-bromo-1-oxo-3,4-dihydroisoquinolin-2(1H)-yl)acetaldehyde (0.35 g, 1.3 mmol), (R)-2-methylpyrrolidine hydrochloride (0.19 g, 1.6 mmol diisopropylethylamine (0.27 mL, 1.6 mmol), 0.37 g (84%) of the title product was obtained as a colorless oil. [α]=−62° (1% solution in Methanol), MS (ES) m/z 337.1 [M+H]⁺.

### Example 9

### Preparation of 6-(4-fluorophenyl)-2-(2-pyrrolidin-1-ylethyl)-3,4-dihydroiso-quinolin-1(2H)-one Hydrochloride

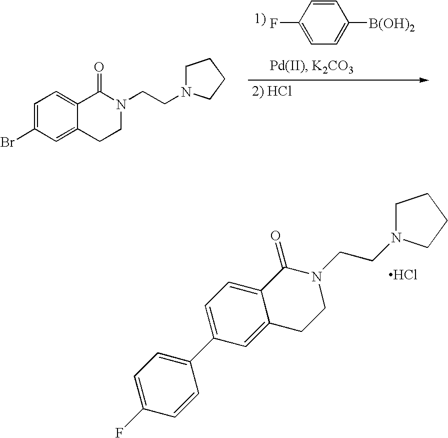

A solution of 6-bromo-2-[2-(pyrrolidin-1-yl)ethyl]-3,4-dihydroisoquinolin-1(2H)-one (0.12 g, 0.37 mmol) and 4-fluorobenzene boronic acid (0.21 g, 1.5 mmol) in dioxane was treated with dichlorobis(tri-o-tolyphosphine)-palladium (II) (14 mg, 0.02 mmol), potassium carbonate (0.17 g, 0.93 mmol) and water, heated to 90° C., stirred for 0.5 h at 90° C., cooled to room temperature and filtered through a pad of celite. The filtrate was partitioned between 1.0 N NaOH and CH₂Cl₂. The aqueous phase was extracted with CH₂Cl₂. The combined organic phases were concentrated in vacuo. The residue was purified by ISCO CombiFlash® chromatography (silica, 0-10% methanol in dichloromethane with 0.5% ammonium hydroxide) to afford the free amine of the title compound as a colorless oil 13 g (77%). The oil was dissolved in ethanol, treated with 1.0 M HCl in diethyl ether, stirred for 10 min. and filtered. The filtercake was washed with diethyl ether and dried to afford the title product as a white solid, mp 207-209° C.; identified by NMR and mass spectral analyses. MS (ES) m/z 339.1; HRMS: calcd for C₂₁H₂₃FN₂O+H⁺, 339.18672; found (ESI, [M+H]⁺), 339.1869.

### Examples 10-28

### Preparation of aryl-2-(2-pyrrolidin-1-ylethyl)-3,4-dihydroisoquinolin-1(2H)-one Hydrochloride Compounds

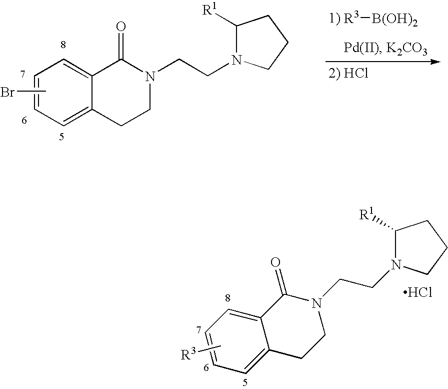

Using essentially the same procedure described in Example 9 and employing the appropriate bromo-2-[2-(pyrrolidin-1-yl)ethyl]-3,4-dihydroisoquinolin-1(2H)-one and the desired aryl boronic acid R³—B(OH)₂, the compounds shown in Table I were obtained and identified by NMR and mass spectral analyses.

TABLE I

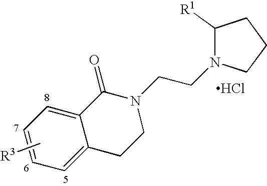

| Ex. No. | R¹ | R³ | mp ° C. | [M + H] |
|---|---|---|---|---|
| 10 | H | 6-(3,5-difluorophenyl) | 196-198 | 357.1 |
| 11 | H | 6-(2,4-difluorophenyl) | 225-226 | 357.2 |
| 12 | H | 6-(2-fluorophenyl) | 211-213 | 339.2 |
| 13 | H | 6-[3-(trifluoromethoxy)phenyl] | 135-137 | 405.1 |
| 14 | H | 6-[4-(trifluoromethoxy)phenyl] | 209-210 | 405.1 |
| 15 | H | 6-(3-cyanophenyl) | 204-206 | 346.2 |
| 16 | H | 6-phenyl | 218-220 | 321.2 |
| 17 | H | 6-(3,4-difluorophenyl) | 179-180 | 357.1 |
| 18 | H | 6-(3-fluorophenyl) | 182-184 | 339.2 |
| 19 | H | 6-(4-cyanophenyl) | 249-251 | 346.2 |
| 20 | H | 6-[3-(trifluoromethyl)phenyl] | 138-139 | 389.1 |
| 21 | H | 6-(1,3-benzodioxol-5-yl) | 249-251 | 365.1 |
| 22 | H | 6-[4-(trifluoromethyl)phenyl] | 243-244 | 389.1 |
| 23 | H | 6-(4-methoxyphenyl) | 224-225 | 351.1 |
| 24 | H | methyl 6-(4-benzoate) | 118-120 | 379.1 |
| 25^{a} | (R)CH₃ | methyl 6-(4-benzoate) | 224-226 | 393.2 |
| 26^{b} | (R)CH₃ | 7-(4-cyanophenyl) | 205-207 | 360.2 |
| 27^{c} | (R)CH₃ | 5-(3-cyanophenyl) | 203-205 | 360.2 |
| 28^{d} | (R)CH₃ | 5-(4-cyanophenyl) | foam | 360.2 |

^{a}[α]_{D}²⁵ = −36.0° (1.00% in Methanol)
^{b}[α]_{D}²⁵ = −3.0° (1.00% in Methanol)
^{c}[α]_{D}²⁵ = −47.0° (1.00% in Methanol)
^{d}[α]_{D}²⁵ = −40.0° (1.00% in Methanol)

### Example 29

### Preparation of 2-(2-(1,3-dioxan-2-yl)ethyl)-6-bromo-3,4-dihydroisoquinolin-1(2H)-one

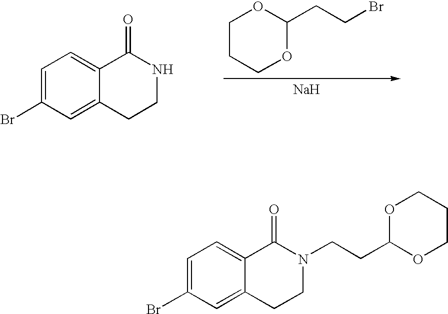

A suspension of sodium hydride (60% dispersion in mineral oil, 0.54 g, 13.6 mmol) in DMF at room temperature, under nitrogen, was treated dropwise over 15 min with a solution of 6-bromo-3,4-dihydroisoquinolin-1(2H)-one (2.05 g, 9.1 mmol) in DMF, stirred at room temperature for 20 min, treated with 2-(2-bromoethyl)-1,3-dioxane (1.84 mL, 13.6 mmol), stirred for 16 h and partitioned between water and CH₂Cl₂. The aqueous phase was extracted with CH₂Cl₂. The combined organic phase and extracts were washed with brine, dried over Na₂SO₄ and concentrated in vacuo. The residue was purified by ISCO CombiFlash® chromatography (silica, 0-100% ethyl acetate in hexanes) to afford the title compound as a light yellow oil, 3.0 g (97%), MS (ES) m/z 340.1 [M+H]⁺.

### Example 30

### Preparation of 3-(6-bromo-1-oxo-3,4-dihydroisoquinolin-2(1H)-yl)propanal

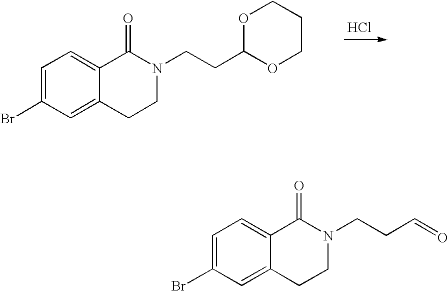

A solution of 2-(2-(1,3-dioxan-2-yl)ethyl)-6-bromo-3,4-dihydroisoquinolin-1(2H)-one (3.0 g, 8.8 mmol) in dioxane was treated dropwise with 12 N HCl (17 mL) at room temperature, heated at 60° C. for 4 h, cooled to room temperature and concentrated in vacuo. The residue was diluted with water and extracted with ethyl acetate. The combined extracts were washed sequentially with brine and water, dried over Na₂SO₄ and concentrated in vacuo. The residue was purified by ISCO CombiFlash® chromatography (silica, 0-10% methanol in methylene chloride) to afford the title compound as an off-white solid, 2.08 g (84%), mp 93-94° C., identified by NMR and mass spectral analyses.

### Example 31

### Preparation of 6-bromo-2-[3-(pyrrolidin-1-yl)propyl]-3,4-dihydroisoquinolin-1(2H)-one

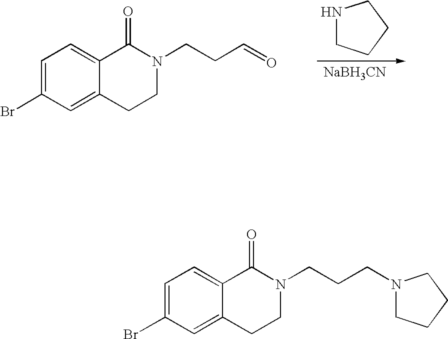

A stirred solution of 3-(6-bromo-1-oxo-3,4-dihydroisoquinolin-2(1H-yl)propanal (0.75 g, 2.7 mmol) and pyrrolidine (0.28 mL, 3.4 mmol) in methanol was treated with sodium cyanoborohydride (0.25 g, 4.0 mmol) and acetic acid (0.38 mL, 6.6 mmol) at room temperature, allowed to stir at room temperature overnight, diluted with 1.0 N NaOH and extracted with CH₂Cl₂. The combined extracts were dried over Na₂SO₄ and concentrated in vacuo. The residue was purified by ISCO CombiFlash® chromatography (silica, 0-10% methanol in methylene with 0.5% ammonium hydroxide) to afford the title product as a colorless oil, 0.48 g (54%), MS (ES) m/z 337.1 [M+H]⁺.

### Examples 32-33

### Preparation of 6-aryl-2-[3-(pyrrolidin-1-yl)propyl]-3,4-dihydroisoquinolin-1(2H)-one Hydrochloride Compounds

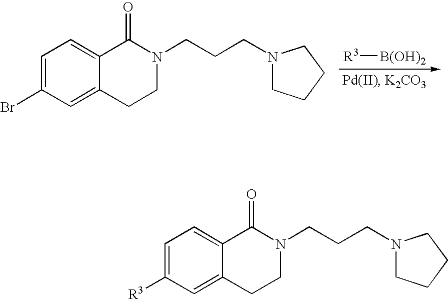

Using essentially the same procedure described in Example 9 and employing 6-bromo-2-[3-(pyrrolidin-1-yl)propyl]-3,4-dihydroisoquinolin-1(2H)-one and the desired aryl boronic acid, the compounds shown in Table II were obtained and identified by NMR and mass spectral analyses.

TABLE II

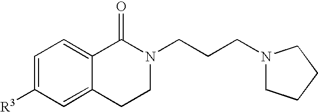

| Ex. | | | |
|---|---|---|---|
| No. | R³ | mp ° C. | [M + H] |
| 32 | 4-cyanophenyl | 192-193 | 360.2 |
| 33 | 3-cyanophenyl | 175-176 | 360.2 |

### Example 34

### Preparation of 6-pyridin-4-yl-2-(2-pyrrolidin-1-ylethyl)-3,4-dihydroisoquinolin-1(2)-one Hydrochloride

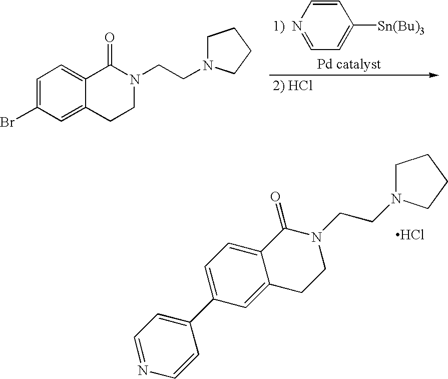

A solution of 6-bromo-2-[2-(pyrrolidin-1-yl)ethyl]-3,4-dihydroisoquinolin-1(2H)-one (0.15 g, 0.46 mmol) and 4-tributylstannyl pyridine (0.68 g, 1.9 mmol) in toluene at 90° C. was treated with tetrakis (triphenylphosphine)palladium (0) (27 mg, 0.02 mmol), stirred at 90° C. for 18 h, cooled to room temperature and filtered through a pad of celite. The filtrate was diluted with 1 N NaOH and extracted with CH₂Cl₂. The combined extracts were concentrated in vacuo. The residue was purified by ISCO CombiFlash® chromatography (silica, 0-10% methanol in dichloromethane with 0.5% ammonium hydroxide) to give the free amine of the title compound as a colorless oil. The oil was dissolved in ethanol, treated with etheral HCl, stirred and filtered. The filtercake was washed with ether and dried to afford the title compound as a white solid, 36 mg, mp 216-218° C.; MS (ES) m/z 322.1; 36 mg HRMS: calcd for C₂₀H₂₃N₃O+H⁺, 322.19139; found (ESI, [M+H]⁺), 322.1926.

### Example 35

### Preparation of 1-[1-oxo-2-(2-pyrrolidin-1-ylethyl)-1,2,3,4-tetrahydroisoquinolin-6-yl]-1H-indole-5-carbonitrile Hydrochloride

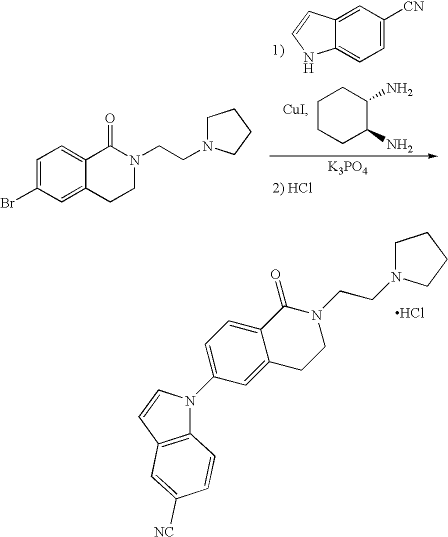

A mixture of 6-bromo-2-[2-(pyrrolidin-1-yl)ethyl]-3,4-dihydroisoquinolin-1(2H)-one (0.1 g, 0.31 mmol), 5-cyanoindole (44 mg, 0.31 mmol), copper (I) iodide (5.9 mg, 0.031 mmol), trans-1,2-cyclohexanediamine (7.1 mg, 0.062 mmol), potassium phosphate (0.14 g, 0.65 mmol) in dioxane was degassed, heated in a CEM microwave for 1 hour at 185° C., cooled to room temperature, diluted with water and extracted with ethyl acetate. The combined extracts were dried over Na₂SO₄ and concentrated in vacuo. The residue was purified by ISCO CombiFlash® chromatography (silica, 0-10% methanol in methylene with 0.5% ammonium hydroxide) to give the free amine of the title product as a colorless oil. The oil was dissolved in ethanol, treated with etheral HCl, stirred and filtered. The filtercake was washed with ether and dried to give the title compound as a white solid, 47.5 mg (37%), mp 215-217° C.; MS (ES) m/z 385.0 [M+H]⁺.

### Example 36

### Preparation of 6-(1H-indazol-1-yl)-2-(2-(pyrrolidin-1-yl)ethyl)-3,4-dihydroiso-quinolin-1(2H)-one hydrochlroride

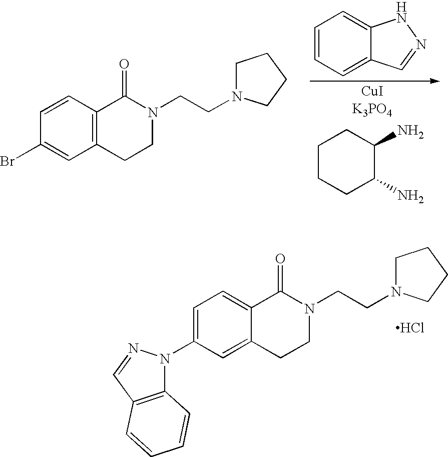

A mixture of 6-bromo-2-[2-(pyrrolidin-1-yl)ethyl]-3,4-dihydroisoquinolin-1(2H)-one (0.08 g, 0.25 mmol), indazole (58 mg, 0.5 mmol), copper (I) iodide (3.5 mg, 0.025 mmol), trans-1,2-cyclohexanediamine (5.6 mg, 0.049 mmol), potassium phosphate (0.11 g, 0.52 mmol) in DMF (120 mL) was heated for 18 hour at 110° C., cooled to room temperature, diluted with water and extracted with ethyl acetate. The combined extracts were dried over Na₂SO₄ and concentrated in vacuo. The residue was purified by ISCO CombiFlash® chromatography (silica, 0-10% methanol in methylene with 0.5% ammonium hydroxide) to give the free amine of the title product as a colorless oil. The oil was dissolved in ethanol, treated with etheral HCl, stirred and filtered. The filtercake was washed with ether and dried to give the title compound 31 mg (43%) as a white solid, mp 185-186° C., HRMS (ES) m/z 361.2027 [M+H]⁺.

### Example 37

### Preparation of 6-(1H-pyrazol-1-yl)-2-(2-(pyrrolidin-1-yl)ethyl)-3,4-dihydroisoquinolin-1(2H)-one hydrochloride

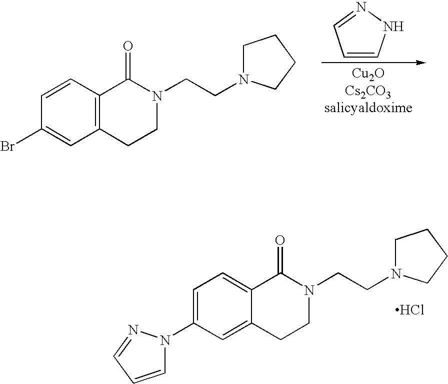

A mixture of 6-bromo-2-[2-(pyrrolidin-1-yl)ethyl]-3,4-dihydroisoquinolin-1(2H)-one (0.10 g, 0.31 mmol), pyrazole (42 mg, 0.62 mmol), copper (I) oxide (4.4 mg, 0.031 mmol), salicyaldoxime (8.5 mg, 0.062 mmol), cesium carbonate (0.2 g, 0.62 mmol) in acetonitrile (15 mL) was heated for 24 hour at 82° C., cooled to room temperature, diluted with water and extracted with ethyl acetate. The combined extracts were dried over Na₂SO₄ and concentrated in vacuo. The residue was purified by ISCO CombiFlash® chromatography (silica, 0-10% methanol in methylene with 0.5% ammonium hydroxide) to give the free amine of the title product as a colorless oil. The oil was dissolved in ethanol, treated with etheral HCl, stirred and filtered. The filtercake was washed with ether and dried to give the title compound 30 mg (66%) as a white solid, mp 174-175° C., HRMS (ES) m/z 311.1869 [M+H]⁺.

### Example 38

### Preparation of 6-(pyrrolidin-1-ylcarbonyl)-2-(2-pyrrolidin-1-ylethyl)-3,4-dihydroisoquinolin-1(2H)-one hydrochloride

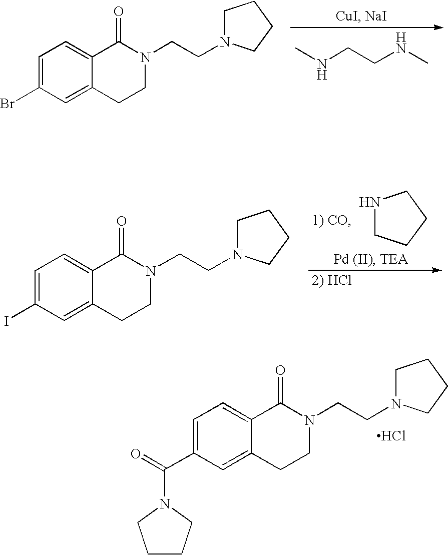

### Step 1: 6-Iodo-2-(2-(Pyrrolidin-1-yl)ethyl)-3,4-dihydroisoquinolin-1(2H)-one

A solution of 6-bromo-2-(2-(pyrrolidin-1-yl)ethyl)-3,4-dihydroisoquinolin-1(2H)-one (0.42 g, 13 mmol), copper (I) iodide (0.25 g, 1.3 mmol), N,N-dimethylethylenediamine (0.03 mL, 2.6 mmol), sodium iodide (0.38 g, 26 mmol), dioxane and DMF was heated in a pressure tube at 80° C. for 2 days. The reaction mixture was cooled to room temperature and filtered through a pad of celite. The filtrate was diluted with water and extracted with CH₂Cl₂. The combined extracts were washed with brine, dried over Na₂SO₄ and concentrated in vacuo. The residue was purified by ISCO CombiFlash® chromatography (silica, 0-10% methanol in methylene with 0.5% ammonium hydroxide) to afford 6-iodo-2-(2-(pyrrolidin-1-yl)ethyl)-3,4-dihydroisoquinolin-1(2H)-one 0.32 g (70%) as a colorless oil, MS (ES) m/z 371 [M+H]⁺; and small amount of 2-(2-pyrrolidin-1-ylethyl)-3,4-dihydroiso-quinolin-1(2H)-one as a clear oil; MS (ESI) m/z 245.1.

### Step 2: 6-(Pyrrolidin-1-ylcarbonyl)-2-(2-pyrrolidin-1-ylethyl)-3,4-dihydro-isoquinolin-1(2H)-one

A mixture of 6-iodo-2-(2-(pyrrolidin-1-yl)ethyl)-3,4-dihydroisoquinolin-1(2H)-one (0.1 g, 0.27 mmol), pyrrolidine (0.44 mL, 5.4 mmol), dichlorobis(tri-phenyl-phosphine)palladium (II) (9 mg, 0.01 mmol), triethylamine (0.13 mL, 0.88 mmol) in DMF was purged with carbon monoxide for 20 minutes, heated in a sealed tube to 90° C. for 16 h, cooled to room temperature and filtered through a pad of celite. The filtrate was diluted with water and extracted with CH₂Cl₂. The combined extracts were washed with brine, dried over Na₂SO₄ and concentrated in vacuo. The residue was purified by ISCO CombiFlash® chromatography (silica, 0-10% methanol in CH₂Cl₂ with 0.5% ammonium hydroxide) to afford the free amine of the title product as a colorless oil. The oil was dissolved in ethanol, treated with etheral HCl, stirred and filtered. The filtercake was washed with ether and dried to provide the title compound as a white solid, 60.8 mg (60%), mp 194-196° C.; MS (ES) m/z 342.2;

HRMS: calcd for C₂₀H₂₇N₃O₂+H⁺, 342.21760; found (ESI, [M+H]⁺), 342.2181.

### Example 39

### Preparation of 6-(4-fluorophenyl)-2-{2-[(2S)-2-methylpyrrolidin-1-yl]ethyl}-3,4-dihydroiso-quinolin-1(2H)-one hydrochloride

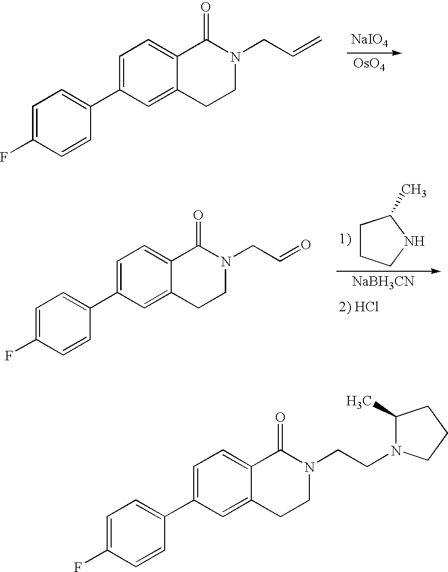

### Step 1: 2-(6-(4-Fluorophenyl)-1-oxo-3,4-dihydroisoquinolin-2(1H)-yl)acetaldehyde

Using essentially the same procedure described in Example 6 and employing 2-allyl-6-(4-fluorophenyl)-3,4-dihydroisoquinolin-1(2H)-one (1.06 g, 3.6 mmol) as starting material the title product was obtained as a colorless oil in 97% yield, identified by NMR and mass spectral analyses.

### Step 2: 6-(4-Fluorophenyl)-2-{2-[(2S)-2-methylpyrrolidin-1-yl]ethyl}-3,4-dihydroiso-quinolin-1(2H)-one

A solution of 2-(6-(4-fluorophenyl)-1-oxo-3,4-dihydroisoquinolin-2(1H)-yl)acetaldehyde (0.1 g, 0.35 mmol) and (S)-2-methylpyrrolidine (0.03 g, 0.35 mmol) in methanol was treated with sodium cyanoborohydride (33 mg, 0.53 mmol) and acetic acid (0.042 mL, 0.88 mmol), stirred at room temperature overnight, diluted with 1 N NaOH and extracted with CH₂Cl₂. The combined extracts were dried over Na₂SO₄ and concentrated in vacuo. The residue was purified by ISCO CombiFlash® chromatography (silica, 0-10% methanol in CH₂Cl₂ with 0.5% ammonium hydroxide) to afford the free amine of the title product as a colorless oil. The oil was dissolved in ethanol, treated with etheral HCl, stirred and filtered. The filtercake was washed with ether and dried to provide the title compound as a white solid, mp 244-247° C., [α]_{D}²⁵=+37.0° (c=1.00 in methanol); MS (ES) m/z 353.1; HRMS: calcd for C₂₂H₂₅FN₂O+H⁺, 353.20237; found (ESI, [M+H]⁺), 353.2024.

### Examples 40-42

### Preparation of 6-(substituted)-2-(2-pyrrolidin-1-ylethyl)-3,4-dihydroiso-quinolin-1(2H)-one hydrochloride compounds

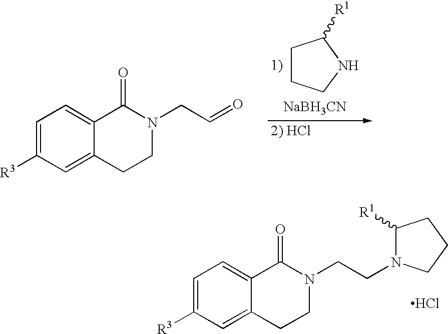

Using essentially the same procedure described in step 2 of Example 39 and employing the appropriate acetaldehyde and the desired pyrrolidine, the compounds shown in Table III were obtained and identified by NMR and mass spectral analyses.

TABLE III

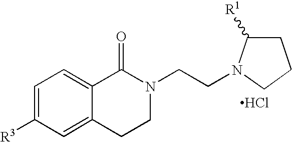

| Ex. No. | R¹ | R³ | mp ° C. | [M + H] | [α]_{D}^{25*} |
|---|---|---|---|---|---|
| 40 | (R)CH₃ | 4-fluorophenyl | 236-238 | 353.1 | −33.0 |
| 41 | (S)CH₃ | 4-cyanophenyl | 268-271 | 360.2 | +39.0 |
| 42 | (R)CH₃ | 4-cyanophenyl | 268-270 | 360.1 | −39.0 |

^{*}1% in methanol

### Example 43

### Preparation of 6-(4-fluorophenyl)-2-(2-piperidin-1-ylethyl)-3,4-dihydroiso-quinolin-1(2H)-one hydrochloride

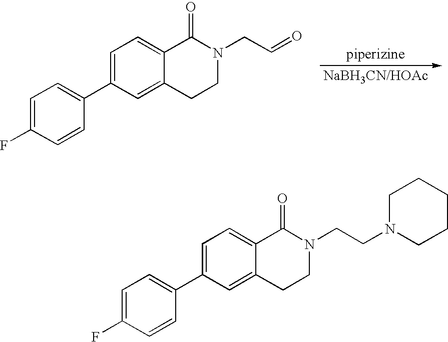

Using essentially the same procedure described in step 2 of Example 39 and employing 2-(6-(4-fluorophenyl)-1-oxo-3,4-dihydroisoquinolin-2(1H)-yl)acetaldehyde (0.1 g, 0.35 mmol) and the piperidine (0.035 mL, 0.35 mmol), the title compound 61 mg (44%) was obtained as a white solid; mp 241-243° c.; HRMS (ES) m/z 353.2025 [M+H]⁺.

### Example 44

### Preparation of 2-(2-azepan-1-ylethyl)-6-(4-fluorophenyl)-3,4-dihydroisoquinolin-1(2H)-one hydrochloride

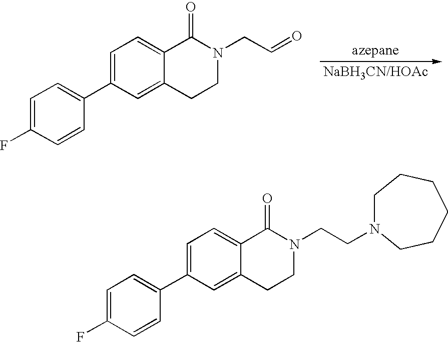

Using essentially the same procedure described in step 2 of Example 39 and employing 2-(6-(4-fluorophenyl)-1-oxo-3,4-dihydroisoquinolin-2(1-yl)acetaldehyde (0.1 g, 0.35 mmol) and piperidine (0.04 mL, 0.35 mmol), the title compound 79 mg (56%) was obtained as a white solid; mp 215-217° C.; HRMS (ES) m/z 367.2181 [M+H]⁺.

### Example 45

### Preparation of 4-[1-oxo-2-(3-piperidin-1-ylpropyl)-1,2,3,4-tetrahydroisoquinolin-6-yl]benzonitrile hydrochloride

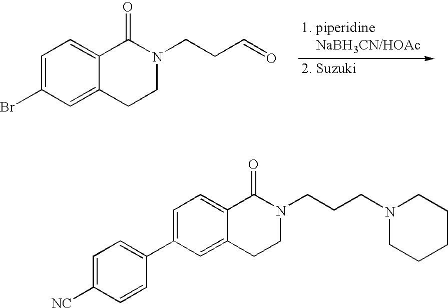

### Step 1: 6-Bromo-2-(3-(Piperidin-1-yl)propyl)-3,4-dihydroisoquinolin-1(2h)-one

Using essentially the same procedure described in Example 31 and employing piperidine (0.12 mL, 1.2 mmol), the title compound 0.26 g (70%) was obtained as a clear oil; MS (ES) m/z 351.0 [M+H]⁺.

### Step 2: 4-[1-oxo-2-(3-piperidin-1-ylpropyl)-1,2,3,4-tetrahydroisoquinolin-6-yl]benzonitrile hydrochloride

Using essentially the same procedure described in Example 9 and employing 6-Bromo-2-(3-(piperidin-1-yl)propyl)-3,4-dihydroisoquinolin-1(2H)-one (0.18 g, 0.5 mmol) and 4-cyanobenzene boronic acid (0.3 g, 2.0 mmol), the title compound 0.1 g (48%) was obtained as a white solid; mp 249-250° C.; HRMS (ES) m/z 374.2232 [M+H]⁺.

### Example 46

### Preparation of 6-(1-oxo-2-(2-oxoethyl)-1,2,3,4-tetrahydroisoquinolin-6-yloxy)nicotinonitrile

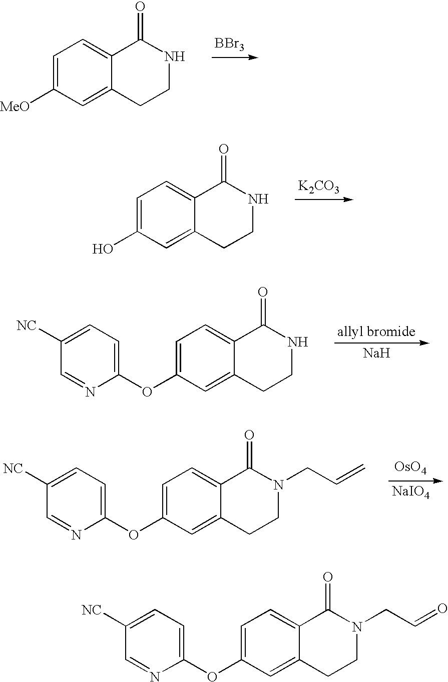

### Step 1: 6-Hydroxy-3,4-dihydroisoquinolin-1(2H)-one

A solution of 6-methoxy-3,4-dihydroisoquinolin-1(2H)-one (2.58 g, 14 mmol) in dichloromethane at −78° C. was treated with boron tribromide (2.7 mL, 28 mmol), allowed to warm to room temperature overnight, quenched with cold water and extracted with ethyl acetate. The combined extracts were concentrated in vacuo. The residue was purified by ISCO CombiFlash® chromatography (silica, 0-15% methanol in dichloromethane) to afford the title compound as a light brown solid, 1.8 g (75%), mp 204-206° C.; MS (ES) m/z 162.1 [M+H]⁺.

### Step 2: 6-(1-Oxo-1,2,3,4-tetrahydroisoquinolin-6-yloxy)nicotinonitrile

A solution of (6-hydroxy-3,4-dihydroisoquinolin-1(2H)-one (0.4 g, 2.4 mmol) and potassium carbonate (0.85 g, 6.0 mmol) in DMF was treated with 2-chloro-pyridine-5-carbonitrile (0.68 g, 4.8 mmol), heated at 90° C. overnight, cooled to room temperature, diluted with water and extracted with CH₂Cl₂. The combined extracts were washed with water, dried over Na₂SO₄ and concentrated in vacuo. The residue was purified by ISCO CombiFlash® chromatography (silica, 0-10% methanol in dichloromethane) to afford the title compound as a white solid, 0.42 g (65%), mp 192-194° C.; MS (ES) m/z 266.1 [M+H]⁺.

### Step 3: 6-(2-Allyl-1-oxo-1,2,3,4-tetrahydroisoquinolin-6-yloxy)-nicotinonitrile

A suspension of sodium hydride (60% dispersion in mineral oil, 0.13 g, 3.2 mmol) in DMF at 0° C. was treated with a solution of 6-(1-oxo-1,2,3,4-tetrahydroiso-quinolin-6-yloxy)nicotinonitrile (0.56 g, 2.1 mmol) in DMF, stirred at 0° C. for 30 minutes, treated with allyl bromide (0.27 mL, 3.2 mmol), stirred at 0° C. for 5 hours, diluted with water and extracted with CH₂Cl₂. The combined extracts were washed with water and concentrated in vacuo. The residue was purified by ISCO CombiFlash® chromatography (silica, 0-5% methanol in dichloromethane) to afford the title product as a colorless oil, 0.53 g (82%), MS (ES) m/z 306.1 [M+H]⁺.

### Step 4: 6-(1-oxo-2-(2-oxoethyl)-1,2,3,4-tetrahydroisoquinolin-6-yloxy)nicotinonitrile

A solution of 6-(2-allyl-1-oxo-1,2,3,4-tetrahydroisoquinolin-6-yloxy)nicotino-nitrile (0.53 g, 1.7 mmol) in THF and water at 0° C. was treated with sodium periodate (1.1 g, 6 mmol), stirred at 0° C. for 10 min, treated with osmium (VIII) tetraoxide (4 wt. % solution in water, 0.75 mL), stirred at 0° C. for 8 h, poured into water and extracted with CH₂Cl₂. The combined extracts were washed with brine, dried over Na₂SO₄ and concentrated in vacuo. The residue was purified by ISCO CombiFlash® chromatography (silica, 0-10% methanol in dichloromethane) to afford the title compound as a colorless oil, 0.40 g (74%), MS (ES) m/z 308.1 [M+H]⁺.

### Examples 47-52

### Preparation of 6-aryloxy-2-(2-pyrrolidin-1-ylethyl)-3,4-dihydroisoquinolin-1(2H)-one hydrochloride compounds

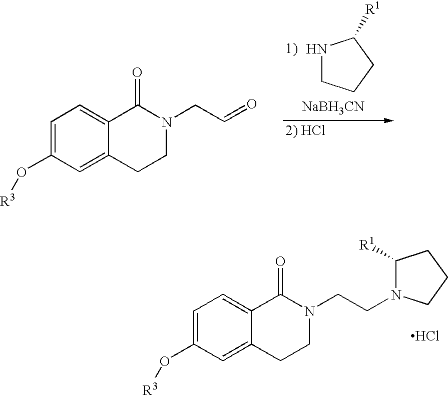

Using essentially the same procedure described in step 2 of Example 39 and employing the appropriate 6-aryloxy-2-[2-(pyrrolidin-1-yl)ethyl]-3,4-dihydroisoquinolin-1(2H)-one and the desired pyrrolidine, the compounds shown in Table IV were obtained and identified by NMR and mass spectral analyses.

TABLE IV

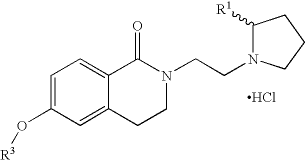

| Ex. | | | | | |
|---|---|---|---|---|---|
| No. | R¹ | R³ | mp ° C. | [M + H] | [α]_{D}²⁵* |
| 47 | H | 5-cyanopyridin-2-yl | 189-190 | 363.1 | — |
| 48 | (R)CH₃ | 5-cyanopyridin-2-yl | 184-185 | 377.2 | −33.0 |
| 49 | (R)CH₃ | 4-cyanophenyl | 254-255 | 376.2 | −33.0 |
| 50 | H | 4-cyanophenyl | 146-147 | 362.2 | — |
| 51 | (R)CH₃ | 6-cyanopyridin-3-yl | 218-220 | 377.2 | −34.0 |
| 52 | H | 6-cyanopyridin-3-yl | 203-205 | 363.2 | — |

*1% in methanol

### Example 53

### Preparation of 4-{1-oxo-2-[2-(pyrrolidine-1-yl)ethyl]-1,2,3,4-tetrahydroiso-quinolin-6-yl}benzoic acid

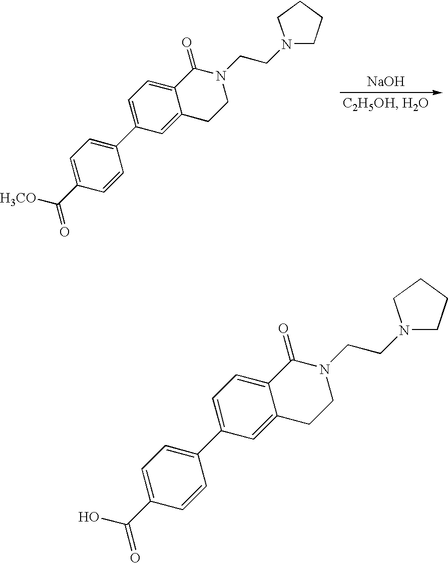

A solution of methyl 4-(1-oxo-2-(2-(pyrrolidin-1-yl)ethyl)-1,2,3,4-tetrahydroiso-quinolin-6-yl)-benzoate (1.74 g, 4.6 mmol) in ethanol at room temperature was treated with a solution of sodium hydroxide (0.36 g, 9.2 mmol) in water, stirred at room temperature for 3 h neutralized to pH 7.0 with 2N HCl and filtered. The filtercake was washed with water and dried under vacuum at 78° C. overnight to afford the title compound as a white solid, 1.60 g (96%), mp 247-249° C.; MS (ES) m/z 365.1 [M+H]⁺.

### Example 54

### Preparation of 6-[4-(pyrrolidin-1-ylcarbonyl)phenyl]-2-(2-pyrrolidin-1-ylethyl)-3,4-dihydroisoquinolin-1(2H)-one hydrochloride

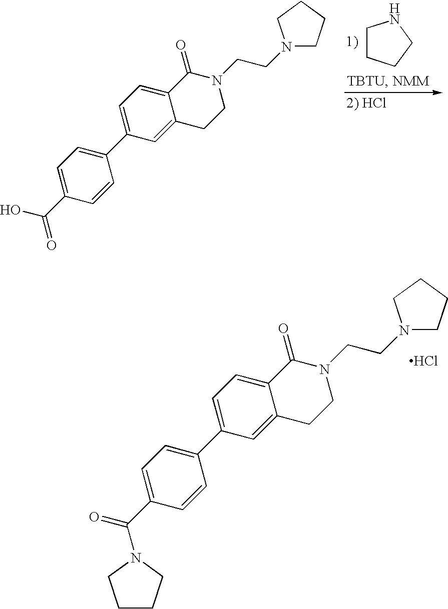

A stirred solution of 4-{1-oxo-2-[2-(pyrrolidine-1-yl)ethyl]-1,2,3,4-tetrahydroiso-quinolin-6-yl}benzoic acid (0.12 g, 0.33 mol) in 1,2-dichloroethane and DMF was treated with 2-(1H-benzotriazol-1-yl)-1,1,3,3-tetramethyluronium tetrafluoroborate (TBTU) (0.13 g, 0.39 mmol), N-methylmorpholine (NMM) (0.18 mL, 1.6 mm) and pyrrolidine (0.03 mL, 0.36 mmol), stirred at room temperature for 3 h, diluted with water and extracted with ethyl acetate. The combined extracts were washed sequentially with saturated NaHCO₃, dried over Na₂SO₄ and concentrated in vacuo. The residue was purified by ISCO CombiFlash® chromatography (silica, 0-10% methanol in CH₂Cl₂ with 0.5% ammonium hydroxide) to afford the free amine of the title compound as a colorless oil. The oil was dissolved in ethanol, treated with etheral HCl, stirred and filtered. The filtercake was washed with ether and dried to provide the title compound as a white solid, 62.5 mg (42%), mp 245-248° C.; MS (ES) m/z 418.1; HRMS: calcd for C₂₆H₃₁N₃O₂+H⁺, 418.24890; found (ESI, [M+H]⁺), 418.2492.

### Example 55

### Preparation of N-cyclopentyl-4-[1-oxo-2-(2-pyrrolidin-1-ylethyl)-1,2,3,4-tetrahydroisoquinolin-6-yl]benzamide

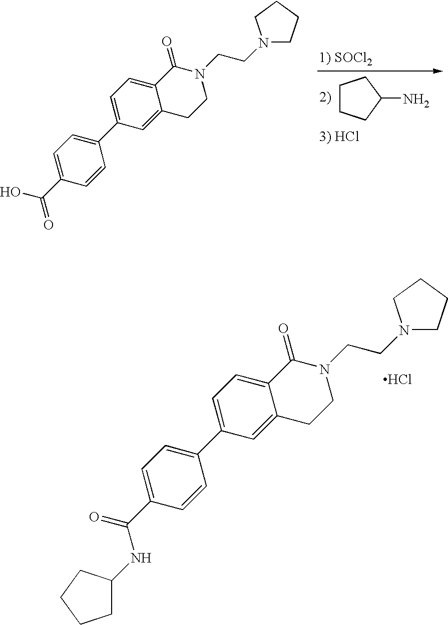

A mixture of thionyl chloride (3 mL) and 4-(1-oxo-2-(2-(pyrrolidin-1-yl)ethyl)-1,2,3,4-tetrahydroisoquinolin-6-yl)benzoic acid (0.10 g, 0.27 mol) was stirred at reflux temperature for 1 h, cooled to room temperature and concentrated in vacuo to afford a solid residue. The solid was dissolved in THF, cooled to 0° C., treated with cylcopentylamine (0.03 mL, 0.3 mmol), warmed to room temperature, stirred for 1 h aat room temperature, diluted with 1 N NaOH and extracted with CH₂Cl₂. The combined extracts were washed sequentially with saturated NaHCO₃ and brine, dried over Na₂SO₄ and concentrated in vacuo. The residue was purified by ISCO CombiFlash® chromatography (silica, 0-10% methanol in CH₂Cl₂ with 0.5% ammonium hydroxide) to afford the free amine of the title product as a colorless oil. The oil was dissolved in ethanol, treated with etheral HCl, stirred and filtered. The filtercake was washed with ether and dried to provide the title compound as a white solid, 56.8 mg (44%), mp 258-260° C.; MS (ES) m/z 432.2; HRMS: calcd for C₂₇H₃₃N₃O₂+H⁺, 432.26455; found (ESI, [M+H]⁺), 432.2649.

### Examples 56-82

### Preparation of N-Substituted-4-[1-oxo-2-(2-pyrrolidin-1-ylethyl)-1,2,3,4-tetrahydroisoquinolin-6-yl]benzamide hydrochloride compounds

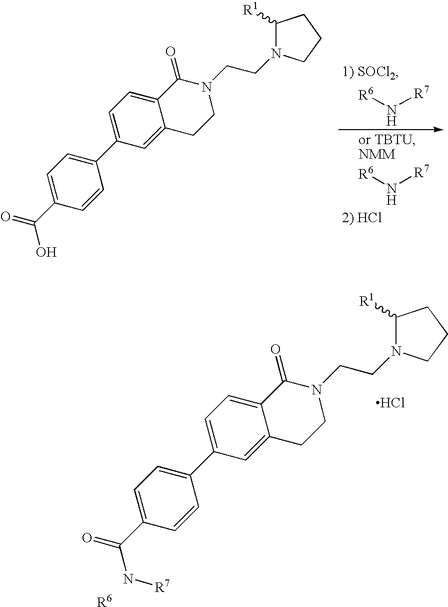

Using essentially the same procedures described in Examples 54 and 55 and employing the appropriate benzoic acid and the desired amine, the compounds shown in Table V were obtained and identified by NMR and mass spectral analyses. For Table V, all optical rotation values were obtained using a 1.0% solution in methanol.

TABLE V

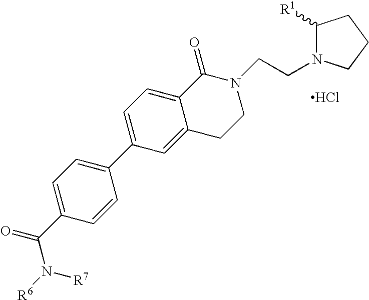

| Ex. | | | | | |
|---|---|---|---|---|---|
| No. | R¹ | NR⁶R⁷ | mp ° C. | [M + H] | [α]_{D}²⁵* |
| 56 | H | dimethylamine | 252-254 | 392.1 | — |
| 57 | H | cyclopropylamine | 222-224 | 404.1 | — |
| 58 | H | ethylamine | 255-257 | 392.1 | — |
| 59 | H | methylamine | 235-236 | 378.1 | — |
| 60 | H | cyclopropylmethylamine | 216-218 | 418.1 | — |
| 61 | H | isopropylamine | 257-259 | 406.1 | — |
| 62 | H | diethylamine | 177-178 | 420.1 | — |
| 63 | H | cyclobutylamine | 255-258 | 418.1 | — |
| 64 | H | azetidine | 229-230 | 404.2 | — |
| 65 | (R)CH₃ | diethylamine | 221-222 | 434.3 | −32 |
| 66 | (R)CH₃ | pyrrolidine | 243-244 | 432.3 | −33 |
| 67 | H | NH₂ | 268-270 | 364.2 | — |
| 68 | H | 2-fluoroethylamine | 215-216 | 410.1 | — |
| 69 | H | 2-methoxyethylamine | 178-180 | 422.1 | — |
| 70 | H | 2-aminoethyl | 164-166 | 450.3 | — |
| | | isopropyl ether | | | |
| 71 | H | 2-phenoxyethylamine | 123-130 | 484.2 | — |
| 72 | H | 2-ethoxyethylamine | 193-195 | 436.2 | — |
| 73 | (R)CH₃ | cyclopropylmethylamine | 188-190 | 432.3 | −28 |
| 74 | (R)CH₃ | cyclobutylamine | 210-211 | 432.3 | −24 |
| 75 | (R)CH₃ | ethylamine | 228-230 | 406.3 | −28 |
| 76 | (R)CH₃ | cyclopropylamine | 255-257 | 418.3 | −31 |
| 77 | (R)CH₃ | isopropylamine | 188-189 | 420.3 | −31 |
| 78 | (R)CH₃ | methylamine | 258-260 | 392.3 | −30 |
| 79 | H | piperidine | 240-241 | 432.2 | — |
| 80 | (R)CH₃ | cyclopentylamine | 260-262 | 446.2 | −31 |
| 81 | H | (S)-2-methylpyrrolidine | 171-172 | 432.2 | 50 |
| 82 | H | (R)-2-methylpyrrolidine | 170-171 | 432.2 | −46 |

*1% in methanol

### Examples 83-86

### Preparation of N-substituted-4-(2-(2-(2-methylpyrrolidin-1-yl)ethyl)-1-oxo-1,2,3,4-tetrahydroisoquinolin-7-yl)benzamide hydrochloride compounds

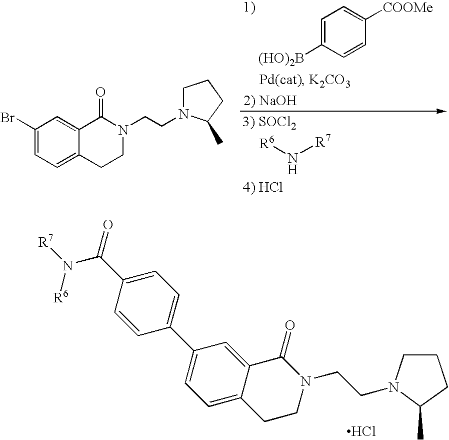

### Step 1: (H)-Methyl 4-(2-(2-(2-methylpyrrolidin-1-yl)ethyl)-1-oxo-1,2,3,4-tetrahydroisoquinolin-7-yl)benzoate

Using essentially the same procedures described in Example 9 and employing (R)-7-bromo-2-[2-(2-methylpyrrolidin-1-yl)ethyl]-3,4-dihydroisoquinolin-1(2H)-one (0.85 g, 2.5 mmol), the title compound 0.8 g (81%) was formed as a white solid, mp 259-260° C., [α]_{D}²⁵=−7°; HRMS (ES) m/z 393.2180 [M+H]⁺. Step 2: (R)-4-(2-(2-(2-Methylpyrrolidin-1-Yl)ethyl)-1-oxo-1,2,3,4-tetrahydroiso-quinolin-7-yl)benzoic Acid

Using essentially the same procedures described in Example 53 and employing (R)-methyl 4-(2-(2-(2-methylpyrrolidin-1-yl)ethyl)-1-oxo-1,2,3,4-tetrahydroisoquinolin-7-yl)benzoate (0.74 g, 1.9 mmol), the title compound 0.63 g (89%) was formed as a white foam, MS (ES) m/z 377.2 [M+H]⁺.

### Step 3: N-Substituted-4-(2-(2-(2-methylpyrrolidin-1-yl)ethyl)-1-oxo-1,2,3,4-tetrahydroisoquinolin-7-yl)benzamide hydrochloride compounds

Using essentially the same procedure described in Example 55 and employing (R)-4-(2-(2-(2-methylpyrrolidin-1-yl)ethyl)-1-oxo-1,2,3,4-tetrahydroiso-quinolin-7-yl)benzoic acid and desired amines, the compounds shown in Table VI were obtained and idnetified by NMR and mass spctral analyses. For Table VI, all optical rotation values were obtained using a 1.0% solution in methanol.

TABLE VI

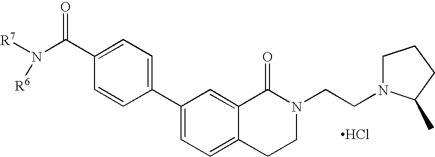

| Ex. | | | | |
|---|---|---|---|---|
| No. | NR⁶R⁷ | mp ° C. | [M + H] | [α]_{D}²⁵* |
| 83 | methylamine | 262-263 | 392.2 | −13 |
| 84 | ethylamine | foam | 406.3 | −7 |
| 85 | isopropylamine | 248-250 | 420.3 | — |
| 86 | pyrrolidine | 214-215 | 432.3 | −5 |

*1% in methanol

### Example 87

### Preparation of 6-methoxy-2-(2-(pyrrolidin-1-yl)ethyl)-3,4-dihydroisoquinolin-1(2H)-one Hydrochloride

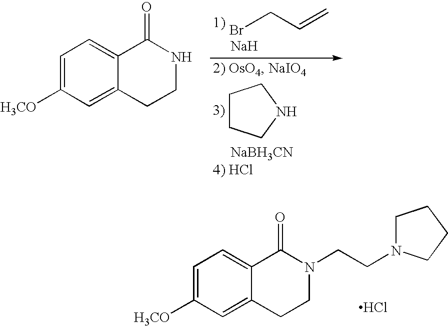

### Step 1: 2-Allyl-6-methoxy-3,4-dihydroisoquinolin-1(2H)-one

Using essentially the same procedures described in Example 3 and and employing 6-methoxy-3,4-dihydroisoquinolin-1(2H)-one (1.0 g, 5.6 mmol), 1.2 g (79%) of 2-allyl-6-methoxy-3,4-dihydroisoquinolin-1(2H)-one was obtained as a light yellow oil. MS (ES) m/z 218.0 [M+H]⁺.

### Step 2: 2-(6-Methoxy-1-oxo-3,4-dihydroisoquinolin-2(1H)-yl)acetaldehyde

Using essentially the same procedures described in Example 6 and employing 2-allyl-6-methoxy-3,4-dihydroisoquinolin-1(2H)-one (1.80 g, 8.3 mmol), 1.27 g (70%) of 2-(6-methoxy-1-oxo-3,4-dihydro-isoquinolin-2(1H)-yl)acetaldehyde was obtained as a white foam, MS (ES) 220.0 [M+H]⁺.

### Step 3: 6-Methoxy-2-(2-(pyrrolidin-1-yl)ethyl)-3,4-dihydroisoquinolin-1(2H)-one

Using essentially the same procedures described in Example 7 and employing 2-(6-methoxy-1-oxo-3,4-dihydroisoquinolin-2(1H)-yl)acetaldehyde (1.26 g, 5.8 mmol), 1.6 g (100%) of 6-methoxy-2-(2-(pyrrolidin-1-yl)ethyl)-3,4-dihydroisoquinolin-1(2H)-one was obtained as a white solid, mp 201-202° C., identified by NMR and mass spectral analyses. HRMS (ES) m/z 275.1756 [M+H]⁺.

### Example 88

### Preparation of 6-hydroxy-2-(2-(pyrrolidin-1-yl)ethyl)-3,4-dihydroisoquinolin-1(2h)-one

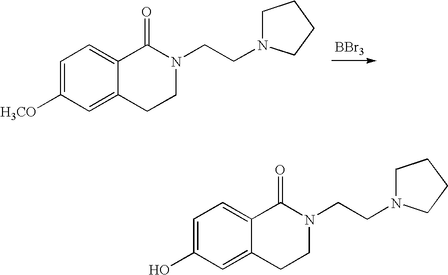

A solution of 6-hydroxy-2-(2-(pyrrolidin-1-yl)ethyl)-3,4-dihydroisoquinolin-1(2H)-one (1.97 g, 7.6 mmol) in dichloromethane at −78° C. was treated with boron tribromide (1.43 mL, 15 mmol), allowed to stir at room temperature overnight, quenched with methanol, neutralized to pH 7 and extracted with methylene chloride. The combined extracts were concentrated in vacuo. The residue was purified by ISCO CombiFlash® chromatography (silica, 0-15% methanol in dichloromethane) to afford the title compound as a light brown solid, 1.75 g (89%), mp 205-207° C.; identified by NMR and mass spectral analyses. MS (ES) m/z 259.2 [M+H]⁺.

### Example 89

### Preparation of methyl 4-(1-oxo-2-(2-(pyrrolidin-1-yl)ethyl)-1,2,3,4-tetrahydro-isoquinolin-6-yloxy)benzoate hydrochloride

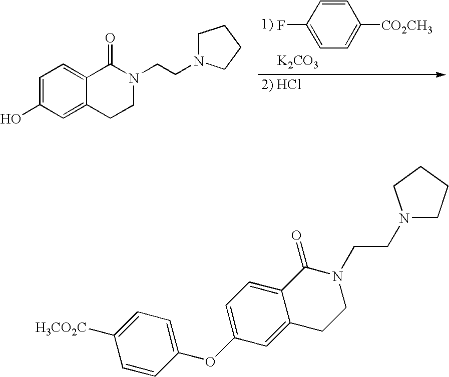

Using essentially the same procedure described in Example 44 and employing 6-hydroxy-2-(2-(pyrrolidin-1-yl)ethyl)-3,4-dihydroisoquinolin-1(2H)-one (1.2 g, 4.9 mmol) and methyl 4-fluorobenzoate (3.1 mL, 24.5 mmol), the title compound was obtained as a white solid, mp 215-216° C.; MS (ES) m/z 395.2 [M+H]⁺.

### Example 90

### Preparation of 4-(1-oxo-2-(2-(pyrrolidin-1-yl)ethyl)-1,2,3,4-tetrahydroisoquinolin-6-yloxy)benzoic acid

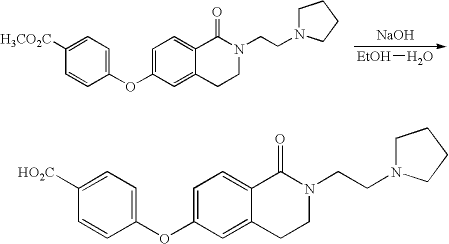

Using essentially the same procedure described in Example 53 and employing methyl 4-(1-oxo-2-(2-(pyrrolidin-1-yl)ethyl)-1,2,3,4-tetrahydro-isoquinolin-6-yloxy)benzoate (0.38 g, 1.3 mmol), the title compound was obtained as a white solid, 0.40 g (74%), mp 99-100° C.; MS (ES) m/z 379.2 [M+H]⁺.

### Examples 91-99

### Preparation of N-substituted-4-(1-oxo-2-(2-(pyrrolidin-1-yl)ethyl)-1,2,3,4-tetrahydro-isoquinolin-6-yloxy)benzamide

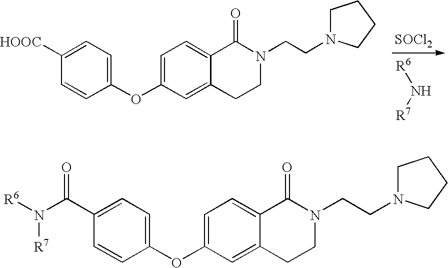

Using essentially the same procedure described in Example 55 and employing 4-(1-oxo-2-(2-(pyrrolidin-1-yl)ethyl)-1,2,3,4-tetrahydroisoquinolin-6-yloxy)benzoic acid and the desired amine, the compounds shown in Table VII were obtained and identified by NMR and mass spectral analyses.

TABLE VII

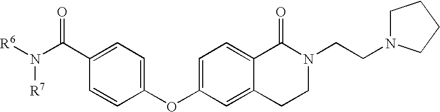

| Ex. No. | NR⁶R⁷ | mp ° C. | [M + H] |
|---|---|---|---|
| 91 | NH2 | foam | 380.2 |
| 92 | methylamine | 235-236 | 392.2 |
| 93 | ethylamine | 215-216 | 408.3 |
| 94 | isopropylamine | 223-224 | 422.3 |
| 95 | N,N-dimethylamine | foam | 408.3 |
| 96 | N,N-diethylamine | foam | 436.3 |
| 97 | cyclobutylamine | 236-237 | 432.3 |
| 98 | pyrrolidine | foam | 434.3 |
| 99 | cyclopropylamine | 227-228 | 420.2 |

### Example 100

### Preparation of 6-chloro-N-methylnicotinamide

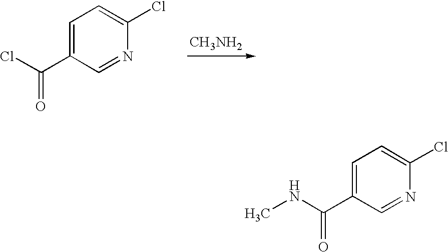

A solution of 6-chloronicotinyl chloride (5.22 g, 30 mmol) in methylene chloride at room temperature was treated with methylamine (2.0 M in THF, 22 mL, 45 mmol), stirred at room temperature for 4 h and filtered. After concentration, the filtrate was filtered. The filtercake was washed with ethyl ether and dried under vacumm to provide the title compound as a white solid, 4.6 g (90%), MS (ES) m/z 169.0 [M−H]⁻.

### Example 101

### Preparation of N-Methyl-6-(1-oxo-2-(2-(pyrrolidin-1-yl)ethyl)-1,2,3,4-tetrahydro-isoquinolin-6-yloxy)nicotinamide hydrochloride

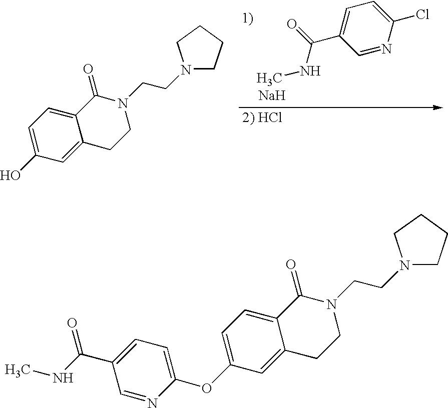

A suspension of NaH (60% dispersion in mineral oil, 0.06 g, 5.4 mmol) in DMF at room temperature was treated dropwise over a 15 min period with a solution of 6-hydroxy-2-(2-(pyrrolidin-1-yl)ethyl)-3,4-dihydroisoquinolin-1(2H)-one (0.2 g, 2.7 mmol) in DMF, stirred at room temperature for 30 min, treated with a solution of 6-chloro-n-methylnicotinamide in DMF, heated at 100° C. overnight, cooled to room temperature, diluted with water and extracted with CH₂Cl₂. The combined extracts were washed with brine, dried over sodium sulfate and concentrated in vacuo. The residue was purified by ISCO CombiFlash® chromatography (silica, 0-15% methanol in methylene plus 0.5% ammonium hydroxide) to afford the free amine of the title product as a colorless oil. The oil was dissolved in ethanol, treated with ethereal HCl, stirred and filtered. The filtercake was washed with ether and dried to provide the title compound as a white solid, 30 mg (10%), mp 228-230° C.; identified by NMR and mass spectral analyses. MS (ES) m/z 395.2 [M+H]⁺.

### Example 102

### Preparation of N-methoxy-N-methyl-4-(1-oxo-2-(2-(pyrrolidin-1-yl)ethyl)-1,2,3,4-tetrahydroisoqui-nolin-6-yl)benzamide

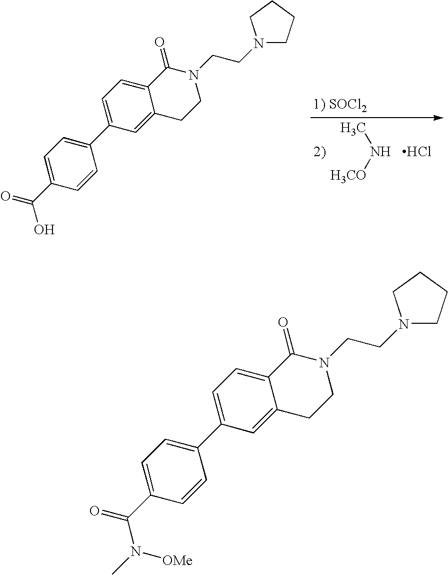

A suspension of 4-(1-oxo-2-(2-(pyrrolidine-1-yl)ethyl)-1,2,3,4-tetrahydroiso-quinolin-6-yl)benzoic acid (0.2 g, 0.55 mmol) in thionyl chloride was heated at reflux temperature for 90 minutes, cooled to room temperature and concentrated in vacuo to afford a residue. The residue was dissolved in CH₂Cl₂ at 0° C., treated with N,O-dimethylhydroxylamine hydrochloride (64 mg, 5.8 mmol), stirred for 30 minutes, treated with triethylamine (0.2 g, 1.4 mmol), allowed to warm to room temperature and stirred overnight. The reaction mixture was diluted with CH₂Cl₂, washed sequentially with saturated NaHCO₃ and brine, dried over Na₂SO₄ and concentrated in vacuo. The resultant residue was purified by ISCO CombiFlash® chromatography (silica, 0-10% methanol in dichloromethane with 0.5% ammonium hydroxide) to give the title compound as a colorless oil, 0.14 g (63%). The oil was dissolved in ethanol and made into its hydrochloride salt as a white solid; mp 190-191° C.; MS (ES) m/z 408.2 [M+H]⁺.

### Example 103

### Preparation of 6-[4-(cyclopropylcarbonyl)phenyl]-2-(2-pyrrolidin-1-ylethyl)-3,4-dihydro-isoquinolin-1(2H)-one

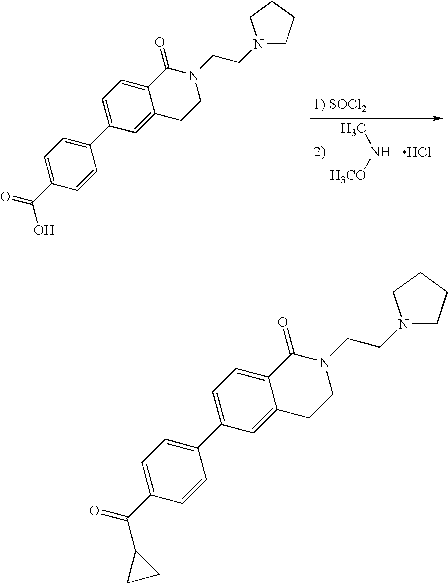

A solution of N-methoxy-N-methyl-4-(1-oxo-2-(2-(pyrrolidin-1-yl)ethyl)-1,2,3,4-tetrahydroisoqui-nolin-6-yl)benzamide(0.14 g, 0.34 mmol) in anhydrous THF at 0° C. was treated with cyclopropyl magnesium bromide (2.0 mL, 0.5 M solution in THF), allowed to warm slowly to room temperature, stirred overnight, quenched with saturated aqueous ammonium chloride and extracted with CH₂Cl₂. The combined extracts were dried over Na₂SO₄ and concentrated in vacuo. The residue was purified by ISCO CombiFlash® chromatography (silica, 0-10% methanol in methylene with 0.5% ammonium hydroxide) to afford the free amine of the title product as a colorless oil. The oil was dissolved in ethanol, treated with etheral HCl, stirred and filtered. The filtercake was washed with ether and dried to provide the title compound as a white solid, 48.4 mg (33%), mp 244-246° C.; MS (ES) m/z 389.2; HRMS: calcd for C₂₅H₂₈N₂O₂+H⁺, 389.22235; found (ESI, [M+H]⁺), 389.2228.

### Example 104

### Preparation of 6-bromo-3,4-dihydro-2-(2-tetrahydro-2H-pyran-2-yloxy)ethyl)iso-quinolin-1-one

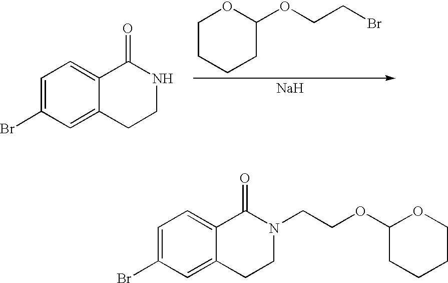

A solution of 6-bromo-3,4-dihydroisoquinolin-1-one (0.36 g, 1.6 mmol) in DMF at 0° C. was treated with sodium hydride (60% dispersion in mineral oil, 0.15 g, 4 mmol), stirred for 1 h, treated with 2-(2-bromoethoxy)tetrahydro-2H-pyran (0.26 mL, 1.7 mmol), allowed to warm to room temperature, stirred overnight, diluted with water and extracted with EtOAc. The combined extracts were washed with water and brine, dried over Na₂SO₄ and concentrated in vacuo. The residue was purified by flash chromatography (silica, petroleum ether/ethyl acetate 2:8) to afford the title compound in 87% yield. ¹H NMR (300 MHz, CDCl₃): 7.92 (d, J=8.1 Hz, 1H); 7.46 (d, J=8.1 Hz, 1H); 7.27 (s, 1H); 4.59 (bs, 1H); 4.02-3.41 (m, 6H); 3.02-2.90 (m, 2H); 1.89-1.39 (m, 8H). LCMS (ESI) m/z 355.5 [M+H]⁺.

### Example 105

### Preparation of 6-benzimidazol-1-yl-2-[2-(tetrahydropyran-2-yloxy)ethyl]-3,4-dihydro-2H-isoqinolin-1-one

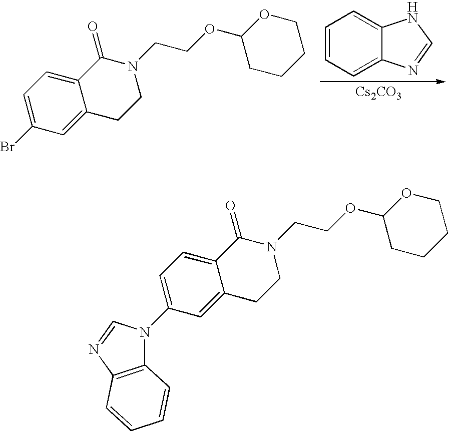

A solution of 6-bromo-3,4-dihydro-2-(2-tetrahydro-2H-pyran-2-yloxy)ethyl)iso-quinolin-1-one (0.354 g, 1.0 mmol) in DMF was treated with cesium carbonate (0.446 g, 2.1 mmol) and benzimidazole (0.141 g, 1.2 mmol), heated at 150° C. for 72 h, cooled to room temperature and diluted with diethyl ether. The phases were separated. The organic phase was washed with water, dried over Na₂SO₄ and concentrated in vacuo. The residue was purified by flash chromatography (silica, petroleum ether/ethyl acetate 1:9 then dichloromethane/methanol 99:1) to afford the title compound in 48% yield. ¹H NMR (300 MHz, CDCl₃): 8.28 (d, J=8.1 Hz, 1H); 8.16 (s, 1H); 7.92-7.84 (m, 1H); 7.71-7.47 (m, 2H); 7.43-7.30 (m, 3H); 4.63 (bs, 1H); 4.06-3.64 (m, 6H)l 3.10 (t, J=6.2 Hz, 2H); 1.89-1.33 (m, 8H). LCMS (ESI) m/z 392.5 [M+H]⁺.

### Example 106

### Preparation of 6-benzimidazol-1-yl-2-[2-(hydroxyethyl]-3,4-dihydro-2H-isoqinolin-1-one

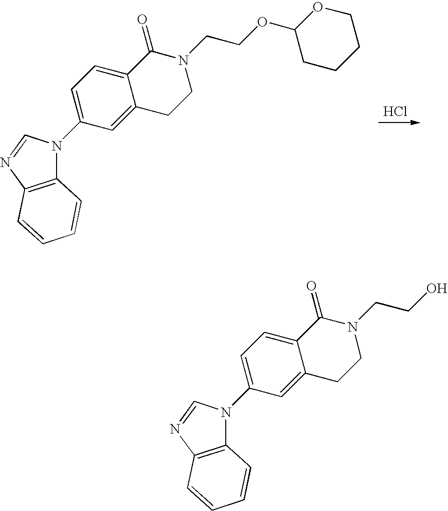

A solution of 6-benzoimidazol-1yl-2-[2-(tetrahydro-pyran-2-yloxy)-ethyl]-3,4-dihydro-2H-isoqinolin-1-one (0.2 g, 0.5 mmol) in ethanol was treated with 12N HCl (0.5 mL), stirred for 3 h, diluted with water and extracted with ethyl acetate. The combined extracts were dried over Na₂SO₄ and concentrated in vacuo. The residue was purified by flash chromatography (petroleum ether/ethyl acetate 2:8) to afford the title compound in 95% yield. ¹H NMR (300 MHz, CDCl₃): 8.6 (d, J=8.6 Hz, 1H); 8.18 (bs, 1H); 7.95-7.88 (m, 1H); 7.66-7.52 (m, 2H); 7.45-7.35(m, 3H); 3.99-3.80 (m, 6H); 3.18 (t, J=6.2 Hz, 2H); 1.64 (s, 1 OH). LCMS ESI m/z 308.4 [M+H]⁺.

### Example 107

### Preparation of 6-(1H-benzimidazol-1-yl)-2-(2-chloroethyl)-3,4-dihydro-2H-isoqinolin-1-one

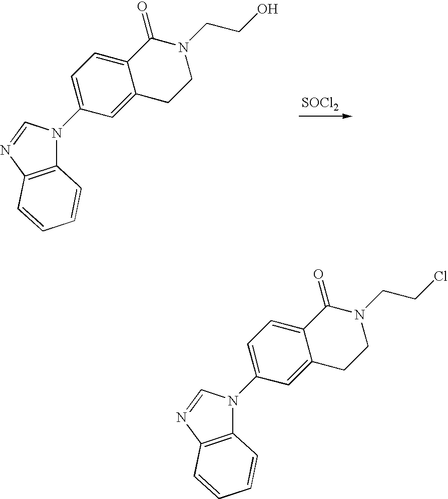

A solution of 6-benzoimidazol-1yl-2-[2-(hydroxy-ethyl]-3,4-dihydro-2H-isoqinolin-1-one (243 mg, 0.79 mmol) in CH₂Cl₂ and DMF was treated with thionyl chloride (0.17 mL, 2.4 mmol), heated at reflux temperature for 15 min, allowed to cool to room temperature and concentrated to dryness in vacuo to give the title compound, LCMS (ESI) m/z 326.4 [M+H]⁺.

### Example 108

### Preparation of 6-(1H-benzimidazol-1-yl)-2-(2-pyrrolidin-1-ylethyl)-3,4-dihydroisoquinolin-1(2H)-one Fumarate Salt

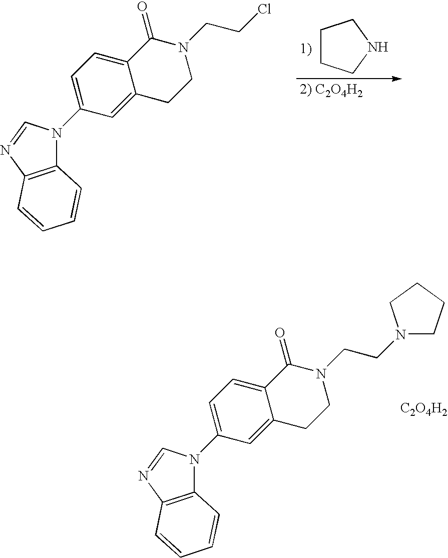

A mixture of 6-(1H-benzimidazol-1yl)-2-(2-chloroethyl)-3,4-dihydro-2H-isoqinolin-1-one (220 mg) and pyrrolidine in a Schlenk tube was heated to 110° C. for 2 h. The excess amine was removed in vacuo. The residue was diluted with ethyl acetate, washed with saturated NaHCO₃, dried over Na₂SO₄ and concentrated in vacuo. The resultant residue was purified by flash column chromatography (silica, dichloromethane:methanol 95:5) to provide the free amine of the title compound as an oil. The oil was dissolved in methylene chloride and methanol, treatedd with fumaric acid, stirred for 30 min. and filtered. The filtercake was washed with ether and dried to provide the title compound as a white solid. ¹H NMR (300 MHz, DMSO-d₆): 8.62 (s, 1H), 8.09 (d, 1H), 7.80 (m, 1H), 7.73 (m, 1H), 7.68 (d, 1H), 7.67 (s, 1H), 7.36 (m, 2 H), 6.57 (s, 2 H), 3.68 (m, 4 H), 3.10 (t, 2 H), 2.82 (t, 2 H), 2.70 (m, 4 H), 1.74 (m, 4 H). LCMS (ESI) m/z 361.3 [M+H]³⁰ .

### Example 109

### Preparation of 5-(benzimidazol-1-ylmethyl)-2-bromobenzaldehyde

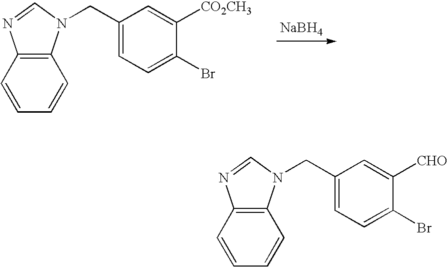

A suspension of methyl 5-(benzimidazol-1-ylmethyl)-2-bromobenzoate (4.0 g, 11.6 mmol) in t-butanol, under nitrogen, was treated with sodium borohydride (0.87 g, 23.2 mmol), heated at reflux temperature overnight, cooled to room temperature and concentrated in vacuo. The residue was dispersed in water and extracted with CH₂Cl₂. The combined extracts were washed with brine, dried over Na₂SO₄ and concentrated in vacuo. The residue was purified by flash column chromatography (silica, dichloromethane/methanol 2.5%) to provide the title compound in 67% yield. LCMS (ESI) m/z 318.3 [M+H]⁺.

### Example 110

### Preparation of [(5-benzimidazol-1-ylmethyl)-2-bromobenzyl]-(2-pyrrolidin-1-yl-ethyl)amine

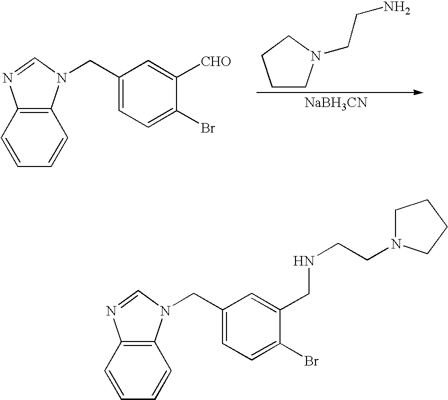

A solution of 5-(benzimidazol-1-ylmethyl)-2-bromobenzaldehyde (1.0 g, 3.2 mmol) in ethanol was treated with 1-(2-aminoethyl)pyrrolidine (439 μL, 3.5 mmol) followed by acetic acid (399 μL, 7 mmol), cooled 0° C., treated with sodium cyanoborohydride (0.29 g, 4.7 mmol), stirred at room temperature overnight and concentrated in vacuo. The residue was dispersed in saturated NaHCO₃ and extracted with CH₂Cl₂. The combined extracts were washed with brine, dried over Na₂SO₄ and concentrated in vacuo. The residue was purified by flash column chromatography (silica, dichloromethane/methanol/ammonium hydroxide 9.5:0.5:0.05) to afford the title compound in 77% yield. (¹H NMR (300 MHz, CDCl₃): 7.96(s, 1H); 7.82(m, 1H); 7.47(d, 1H); 7.36(d, 1H); 7.27(m, 3H); 6.89(dd, 1H); 5.32(s, 2H); 3.84(s, 2H); 2.79-2.46(m, 8H); 1.81(m, 4H). LCMS (ESI) m/z 414.3 [M+H]⁺.

### Example 111

### Preparation of [(5-benzimidazol-1-ylmethyl)-2-bromobenzyl]-(2-pyrrolidin-1-yl-ethyl)carbamic acid methyl ester

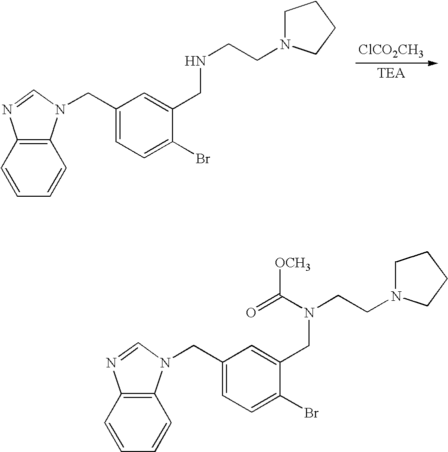

A solution of [(5-benzimidazol-1-ylmethyl)-2-bromobenzyl]-(2-pyrrolidin-1-yl-ethyl)amine (0.285 g, 0.69 mmol) and triethylamine(TEA) (114 μL, 0.82 mmol) in CH₂Cl₂, under nitrogen, at −5° C. was treated with methylchloroformate (54 μL, 0.69 mmol) over a 15 min period, stirred at −5° C. for 1 h and concentrated in vacuo. The residue was dispersed in saturated NaHCO₃ and extracted with CH₂Cl₂. The combined extracts were washed with brine, dried over Na₂SO₄ and concentrated in vacuo. The resultant residue was purified by flash column chromatography (dichloromethane/methanol 9:1) to give the title compound in 77% yield. LCMS (ESI) m/z 472.4 [M +H]⁺.

### Example 112

### Preparation of 5-(1H-benzimidazol-1-ylmethyl)-2-(2-pyrrolidin-1-ylethyl)iso-indolin-1-one

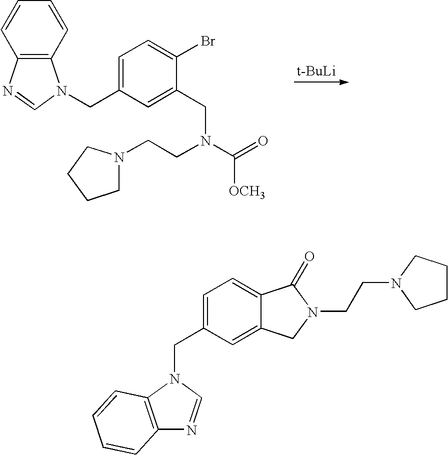

A solution of [(5-benzimidazol-1-ylmethyl)-2-bromobenzyl]-(2-pyrrolidin-1-yl-ethyl)carbamic acid methyl ester (0.095 g, 0.2 mmol) in THF, under nitrogen, at −90° C. was treated dropwise with t-butyllithium (298 μL, 1.5 M in pentane), allowed to come to room temperature, stirred at room temperature overnight and concentrated under reduced pressure. The residue was dispersed in 5% NaHCO₃ and extracted with CH₂Cl₂. The combined extracts were washed with brine, dried over Na₂SO₄ and concentrated in vacuo. The residue was purified (silica, dichloromethane:methanol 8:2) to afford the title product in 15% yield, identified by NMR and mass spectral analyses. LCMS (ESI) m/z 361.3 [M+H]⁺.

### Example 113

### Preparation of methyl 4-Bromo-2-(bromomethyl)benzoate

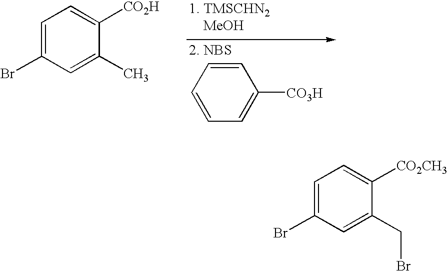

### Step 1: Methyl 4-bromo-2-methylbenzoate

A stirred suspension of 4-bromo-2-methylbenzoic acid (4.98 g, 23 mmol) in dichloromethane (80 mL) and methanol (15 mL) was treated carefully with a 2.0 M solution of trimethylsilyldiazomethane (11.6 mL, 28 mmol) at 0° C. The resulting solution was stirred at 0° C. for 3 hour. The mixture was partitioned between dichloromethane and 1 N sodium hydroxide. The combined organic phases were concentrated under reduced pressure and the residue was purified by ISCO CombiFlash® chromatography (0-5% ethyl acetate in hexanes) to afford 4.72 g (89%) methyl 4-bromo-2-methylbenzoate as a colorless oil. MS (El) 228 [M]⁺.

### Step 2: Methyl 4-Bromo-2-(bromomethyl)benzoate

A solution of methyl 4-bromo-2-methylbenzoate (1.0 g, 4.3 mmol) in CCl₄ was treated with N-bromosuccinimide (0.93 g, 5.2 mmol) and benzoyl peroxide (0.53 g, 2.2 mmol), heated at 85° C. for 5 h, cooled to room temperature and filtered The filtercake was washed with CCl₄ and the filtrates were combined and concentrated in vacuo to provide an oil residue. The residue was purified by ISCO CombiFlash® chromatography (silica, 0-5% ethyl acetate in hexanes) to afford the title compound, 1.59 g (74%), MS (EI) m/z 308 [M]⁺.

### Example 114

### Preparation of 2-allyl-5-bromoisoindolin-1-one

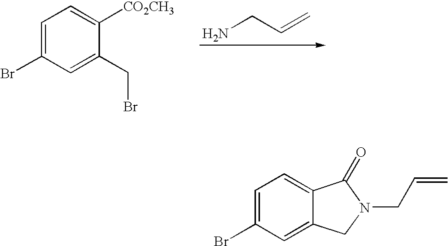

A mixture of methyl 4-bromo-2-(bromomethyl)benzoate (4.19 g, 13.5 mmol) and allyl amine (20 mL) was heated at 50° C. for 12 hours, cooled to room temperature, diluted with CH2Cl2, washed sequentially with 1.0 N HCl and brine, dried over Na₂SO₄ and concentrated in vacuo. The residue was purified by ISCO CombiFlash® chromatography (silica, 0-75% ethyl acetate in hexanes) to afford 2.13 g (62%) of the title compound as a white solid, mp 58-60° C.; MS (ES) m/z 252.0 [M+H]⁺.

### Example 115

### Preparation of 2-(5-bromo-1-oxoisoindolin-2-yl)acetaldehyde

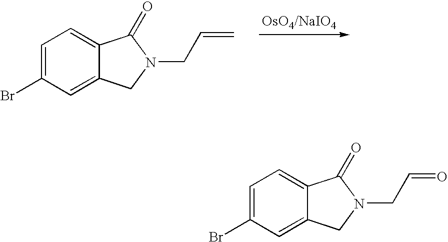

Using essentially the same procedure described in Example 6 and employing 2-allyl-5-bromoisoindolin-1-one (2.13 g, 8.4 mmol), the title compound was obtained as a light yellow oil, 1.34 g (62%), MS (ES) m/z 254.0 [M+H]⁺.

### Example 116

### Preparation of (R)-5-bromo-2-[2-(2-methylpyrrolidin-1-yl)ethyl]isoindolin-1-one

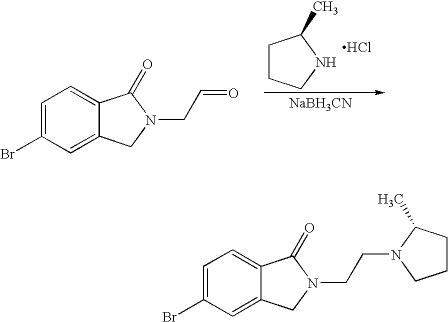

Using essentially the same procedure described in Example 7 and employing 2-(5-bromo-1-oxo-3,4-dihydroisoquinolin-2(1H)-yl)acetaldehyde (0.35 g, 13 mmol), (R)-2-methylpyrrolidine hydrochloride (0.19 g, 15.6 mmol) and diisopropylethylamine (0.34 mL, 15.6 mmol), the title compound was obtained as a colorless oil, 0.37 g (84%), [α]_{D}²⁵=−62° (c=1.00 in methanol); MS (ES) m/z 337.1 [M+H]⁺.

### Example 117-119

### Preparation of (R)-4-(2-(2-(2-methylpyrrolidin-1-yl)ethyl)-1-oxoisoindolin-5-yl)benzonitrile

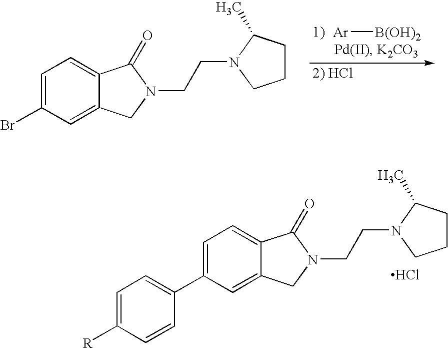

Using essentially the same procedure described in Example 9 and employing (R)-5-bromo-2-[2-(2-methylpyrrolidin-1-yl)ethyl]isoindolin-1-one and desired boronic acids, the compounds shown in Table VIII were obtained and identified by NMR and mass spectral analyses.

TABLE VIII

| Ex. | | | | |
|---|---|---|---|---|
| No | ArB(OH)2 | [α]D25* | mp | [M + H]+ |
| 117 | 4-cyano phenyl-boronic | −54 | 214-216° C. | 346.1. |
| | acid | | | |
| 118 | 4-(methylcarbamoyl)- | −56 | foam | 378.2176 |
| | phenylboronic acid | | | |
| 119 | 4-(pyrrolidine-1-carbonyl)- | −38.0 | 184-185 | 418.2489 |
| | phenylboronic acid | | | |

*c = 1.00% in methanol),

### Example 120

### Preparation of (R)-4-((2-(2-(2-Methylpyrrolidin-1-yl)ethyl)-1-oxo-1,2,3,4-tetra-hydroisoquinolin-6-yloxy)methyl)benzonitrile hydrochloride

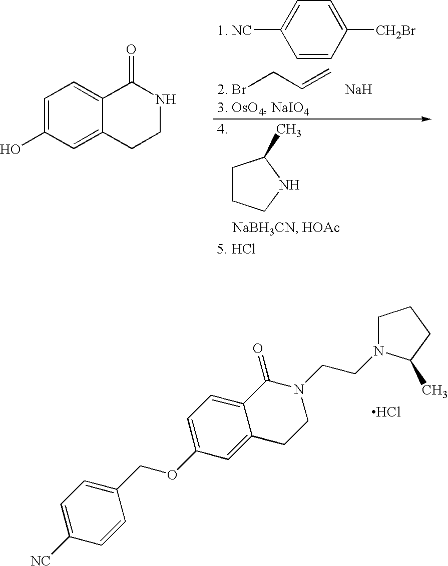

### Step 1: 4-((1-Oxo-1,2,3,4-tetrahydroisoquinolin-6-yloxy)methyl)-benzonitrile

A mixture of 6-hydroxy-3,4-dihydroisoquinolin-1(2H)-one (0.46 g, 2.8 mmol), potassium carbonate(0.97 g, 7 mmol) and 4-(bromomethyl)benzonitrile (0.83 g, 4.2 mmol) in DMF was stirred at room temperature overnight, diluted with water and extracted with methylene chloride. The combined extracts were washed with water, dried over sodium sulfate and concentrated in vacuo. The residue was purified by ISCO CombiFlash® chromatography (silica, 0-10% methanol in methylene chloride) to afford the title compound as a white solid, 0.3 g (38%), mp 158-160° C.; MS (ES) m/z 279.1 [M+H]⁺.

### Step 2: 4-((2-Allyl-1-oxo-1,2,3,4-tetrahydroisoquinolin-6-yloxy)methyl)-benzonitrile

A suspension of NaH (60% dispersion in mineral oil, 95 mg, 2.4 mmol) in DMF at room temperature was treated with a solution of 4-((1-oxo-1,2,3,4-tetrahy-droisoquinolin-6-yloxy)methyl)benzonitrile (0.40 g, 1.6 mmol) in DMF, heated at 65° C. for 10 min, cooled to 0° C., treated with allyl bromide (0.18 mL, 2.1 mmol), stirred at 0° C. for 10 min, diluted with water and extracted with CH₂Cl₂. The combined extracts were washed with water and concentrated in vacuo. The residue was purified by ISCO CombiFlash® chromatography (silica, 0-5% methanol in dichloromethane) to afford the title compound as a colorless oil, 0.15 g (30%), MS (ES) m/z 319.2 [M+H]⁺.

### Step 3: 4-{[1-Oxo-2-(2-oxoethyl)-1 2,3,4-tetrahydroisoquinolin-6-yloxy]-methyl}benzonitrile

Using essentially the same procedure described in Example 46 and employing 4-((2-allyl-1-oxo-1,2,3,4-tetrahydroisoquinolin-6-yloxy)methyl)benzonitrile (0.15 g, 0.47 mmol), the title compound was obtained as a colorless oil.

### Step 4: (R)-4-((2-(2-(2-Methylpyrrolidin-1-yl)ethyl)-1-oxo-1,2,3,4-tetra-hydroisoquinolin-6-yloxy)methyl)benzonitrile hydrochloride

Using essentially the same procedure described in Example 8 and employing 4-{[1-oxo-2-(2-oxoethyl)-1,2,3,4-tetrahydroisoquinolin-6-yloxy]-methyl}benzonitrile (0.16 g, 0.5 mmol) and (R)-2-methyl pyrrolidine, the title compound was obtained as a white solid, mp 212-214° C., [α]_{D}²⁵=−33° (c=1.00% in methanol), identified by NMR and mass spectral analyses. MS (ES) m/z 390.2 [M+H]⁺.

### Examples 121-130

### Preparation of N-substituted-4-[1-oxo-2-(2-pyrrolidin-1-ylethyl)-1,2,3,4-tetrahydroisoquinolin-6-yl]benzamide hydrochloride compounds

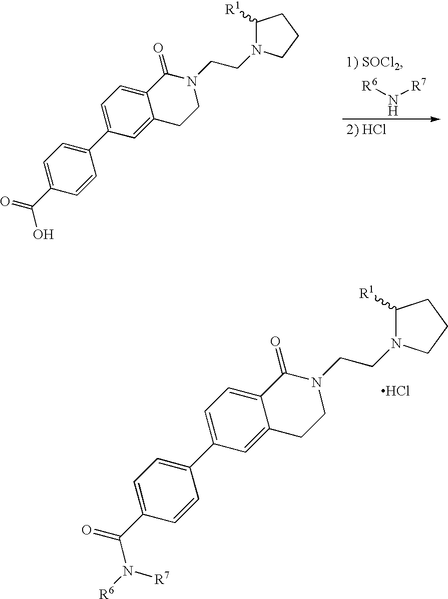

Using essentially the same procedure described in Example 55 and employing the appropriate benzoic acid and the desired amine, the compounds shown in Table V were obtained and identified by NMR and high resolution mass spectral analyses. For Table IX, all optical rotation values were obtained using a 1.0% solution in methanol.

TABLE IX

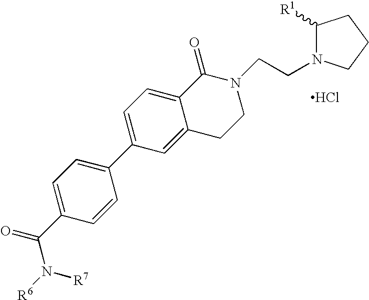

| Ex. | | | | | |
|---|---|---|---|---|---|
| No. | R¹ | NR⁶R⁷ | mp ° C. | [M + H] | [α]_{D}²⁵ |
| 121 | H | thiophen-2-ylmethanamine | 211-213.5 | 460.2057 | — |
| 122 | H | morpholine | 272-274 | 434.2438 | — |
| 123 | H | 2-chloroethanamine | 236-237 | 426.1942 | — |
| 124 | H | N-methylethanamine | 200-201 | 406.2489 | — |
| 125 | H | furan-2-ylmethanamine | 211-213 | 444.2283 | — |
| 126 | H | (S)-1-methoxypropan-2- | 232-233 | 436.2596 | +12 |
| | | amine | | | |
| 127 | H | 3-methoxypyrrolidine | foam | 448.2595 | — |
| 128 | H | (S)-2- | 193-195 | 462.2752 | −64 |
| | | (methoxymethyl)- | | | |
| | | pyrrolidine | | | |
| 129 | H | propylamine | 240-241 | 406.2491 | — |
| 130 | H | thiazol-2-amine | 284-285 | 447.1850 | — |

1% solution in MeOH

### Example 131-135

### Preparation of 6-aryl-2-(2-(pyrrolidin-1-yl)ethyl)-3,4-dihydroisoquinolin-1(2H)-one

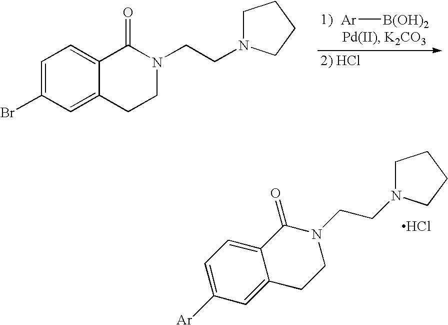

Using essentially the same procedure described in Example 9 and employing the appropriate bromo-2-[2-(pyrrolidin-1-yl)ethyl]-3,4-dihydroisoquinolin-1(2H)-one and the desired aryl boronic acid Ar—B(OH)₂, the compounds shown in Table X were obtained and identified by NMR and high resolution mass spectral analyse

TABLE X

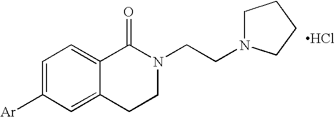

| Ex. No. | Ar—B(OH)₂ | mp ° C. | [M + H] |
|---|---|---|---|
| 131 | 4-fluoro-3-(pyrrolidine-1- | foam | 436.2399 |
| | carbonyl)phenylboronic acid | | |
| 132 | 3-(dimethylcarbamoyl)-4- | foam | 410.2238 |
| | fluorophenylboronic acid | | |
| 133 | 3-fluoro-4-(pyrrolidine-1- | 231-232 | 436.2409 |
| | carbonyl)phenylboronic acid | | |
| 134 | 3-chloro-4-(pyrrolidine-1- | foam | 452.2095 |
| | carbonyl)phenylboronic acid | | |
| 135 | 4-(methylsulfonyl) | 153-155 | 399.1737 |
| | phenylboronic acid | | |

### Example 136

### Preparation of 3-fluoro-4-(1-oxo-2-(2-(pyrrolidin-1-yl)ethyl)-1,2,3,4-tetrahydrois-quinolin-6-yl)benzoic acid

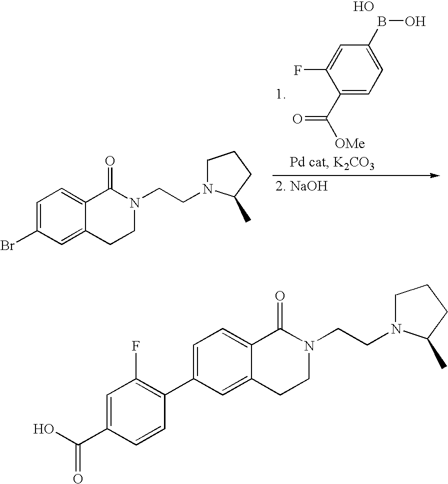

### Step 1: Methyl 3-fluoro-4-(1-oxo-2-(2-(pyrrolidin-1-yl)ethyl)-1,2,3,4-tetrahydro-isoquinolin-6-yl)benzoate

Using essentially the same procedure described in Example 9 and employing 6-bromo-2-(2-(pyrrolidin-1-yl)ethyl)-3,4-dihydroisoquinolin-1(2H)-one (0.36 g, 1.1 mmol) and 2-fluoro-4-(methoxycarbonyl)phenylboronic acid, the title product 0.14 g (32%) was obtained as a light yellow oil, [α]_{D}²⁵=−16° (c=1.00 in methanol);HRMS (ES) m/z 411.2074 [M+H]⁺.

### Step 2: 3-Fluoro-4-(1-oxo-2-(2-(pyrrolidin-1-yl)ethyl)-1 2,3,4-tetrahydroiso-quinolin-6-yl)benzoic acid

Using essentially the same procedure described in Example 53 and employing methyl 3-fluoro-4-(1-oxo-2-(2-(pyrrolidin-1-yl)ethyl)-1,2,3,4-tetrahydro-isoquinolin-6-yl)benzoate (0.14 g, 0.35 mmol), the title compound 0.13 g (99%) was obtained as a white foam, [α]_{D}²⁵=−44° (c=1.00 in methanol);MS (ES) m/z 397.2 [M+H]⁺.

### Example 137-140

### Preparation of 3-fluoro-N-substituted-4-(1-oxo-2-(2-(pyrrolidin-1-yl)ethyl)-1,2,3,4-tetrahydroisoquinolin-6-yl)benzamide hydrochloride compounds

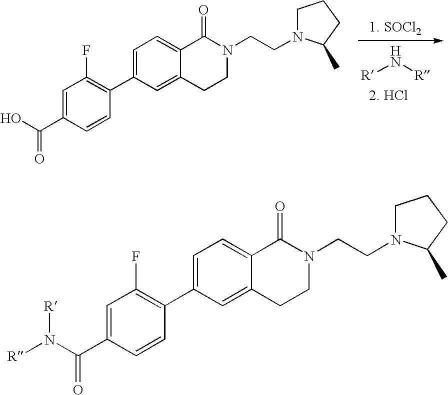

Using essentially the same procedure described in Example 55 and employing 3-fluoro-4-(1-oxo-2-(2-(pyrrolidin-1-yl)ethyl)-1,2,3,4-tetrahydroiso-quinolin-6-yl)benzoic acid and the desired amine, the compounds shown in Table XI were obtained and identified by NMR and high resolution mass spectral analyses.

TABLE XI

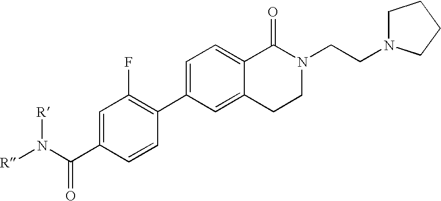

| Ex. No. | R″R′NH | mp ° C. | [M + H] |
|---|---|---|---|
| 137 | NHMe₂ | 228-229 | 410.2240 |
| 138 | pyrrolidine | 225-226 | 436.2395 |
| 139 | NHMe | 188-190 | 396.2090 |
| 140 | NHEt | 209-210 | 410.2245 |

### Example 141-142

### Preparation of N-substituted-4-(1-oxo-2-(3-(pyrrolidin-1-yl)propyl)-1,2,3,4-tetrahydro-isoquinolin-6-yl)benzamide hydrochloride compounds

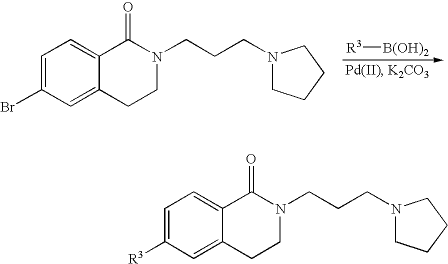

Using essentially the same procedure described in Example 9 and employing 6-bromo-2-[3-(pyrrolidin-1-yl)propyl]-3,4-dihydroisoquinolin-1(2-one and the desired aryl boronic acids, the compounds shown in Table XII were obtained and identified by NMR and high resolution mass spectral analyses.

TABLE XII

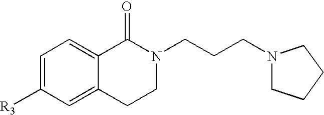

| Ex. No. | R³ | mp ° C. | [M + H] |
|---|---|---|---|
| 141 | 4-(methylcarbamoyl)- | 158-159 | 392.2338 |
| | phenylboronic acid | | |
| 142 | 4-(pyrrolidine-1-carbonyl)- | 223-224 | 432.2652 |
| | phenylboronic acid | | |

### Example 143

### Preparation of (R)-6-iodo-2-(2-(2-methylpyrrolidin-1-yl)ethyl)-3,4-dihydroiso-quinolin-1(2H)-one

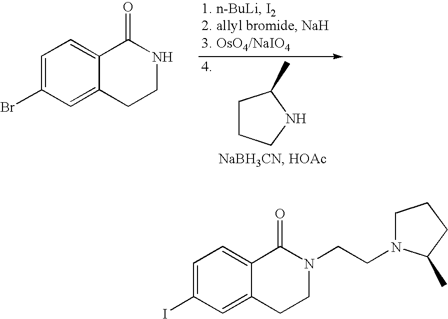

### Step 1: 6-Iodo-3,4-dihydroisoquinolin-1(2H)-one

A solution of 6-bromo-3,4-dihydroisoquinolin-1(2H)-one (15 g, 66.35 mol) in dry THF (1.0 L) cooled to −78° C. and added n-butyl lithium (1.1 M, 151 mL, 166 mol) slowly into the reaction mixture. The reaction mixture was stirred at −78° C. for 1 h. Then iodine (67.1 g, 265.4 mol) in THF was slowly added dropwise at −78° C. The reaction mixture was stirred for another 1.5 h at −78° C. The reaction mixture was quenched with ammonium chloride solution and extracted with ethyl acetate & washed with water, brain solutions and dried over sodium sulfate and concentrated in vacuo. The crude product was purified by column chromatography (Silica, 1-75% ethyl acetate in hexane) to afford 8.15 g (45%) of the title product as a white solid, mp>300° C.; (ES) m/z 273.1 [M +H]⁺.

### Step 2: 2-Allyl-6-iodo-3,4-dihydroisoquinolin-1(2H)-one

According to the procedure described for 3a, starting from 6-iodo-3,4-dihydroisoquinolin-1(2H)-one (4.75 g, 17 mmol), 4.3 g (79%) of 2-allyl-6-iodo-3,4-dihydroisoquinolin-1(2H)-one was obtained as a light yellow oil. HRMS (ES) m/z 314.0043 [M+H]⁺.

### Step 3: 2-(6-Iodo-1-oxo-3,4-dihydroisoquinolin-2(1H)-yl)acetaldehyde

According to the procedure described for 6a, starting from 2-allyl-6-iodo-3,4-dihydroisoquinolin-1(2H)-one (5.05 g, 16 mmol), 4.0 g (79%) of the title product was obtained as a white foam. HRMS (ES) m/z 315.9827 [M+H]⁺.

### Step 4: (R)-6-Iodo-2-(2-(2-methylpyrrolidin-1-yl)ethyl)-3,4-dihydroisoquinolin-1(2H-one

Using essentially the same procedure described in Example 8a and employing 2-(6-iodo-1-oxo-3,4-dihydroisoquinolin-2(1H)-yl)acetaldehyde (2.0 g, 6.3 mmol), the title compound was obtained as a colorless oil, 1.94 g (80%), [α]_{D}²⁵=−36° (c=1.00 in methanol); HRMS (ES) m/z 385.0773 [M+H]⁺.

### Example 144-153

### Preparation of (R)-N,N-substituted-2-(2-(2-methylpyrrolidin-1-yl)ethyl)-1-oxo-1,2,3,4-tetrahydroisoquinoline-6-carboxamide hydrochloride compounds

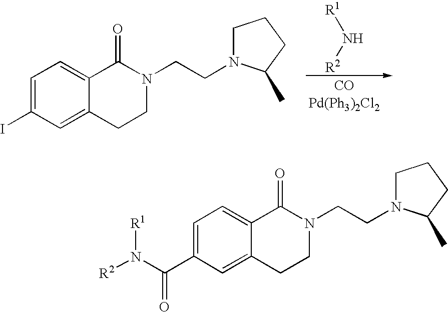

Using essentially the same procedure described in step 2 of Example 38 and employing (R)-6-iodo-2-(2-(2-methylpyrrolidin-1-yl)ethyl)-3,4-dihydroisoquinolin-1(2H)-one and the desired amines as the starting materials, the compounds shown in Table XIII were obtained and identified by NMR and high resolution mass spectral analyses.

TABLE XIII

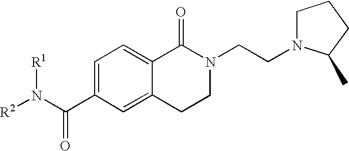

| Ex. No. | NHR¹R² | [α₁]_{D}²⁵* | mp ° C. | [M + H] |
|---|---|---|---|---|
| 144 | azepane | −29 | 192-193 | 384.2649 |
| 145 | Cyclobutyl amine | −32 | 214-215 | 356.2338 |
| 146 | piperidine | −31 | foam | 370.2492 |
| 147 | cyclohexylamine | −28 | 140-141 | 384.2648 |
| 148 | 2,3-dihydro-1H- | −31 | 145-146 | 418.2493 |
| | inden-2-amine | | | |
| 149 | cyclopentylamine | −25 | 219-220 | 370.2490 |
| 150 | 1,2,3,4- | −26 | foam | 418.2493 |
| | tetrahydroisoquinoline | | | |
| 151 | pyrrolidine | −33 | 166-167 | 356.2335 |
| 152 | isoindoline | −29 | 109-110 | 404.2338 |
| 153 | pyridine-4-amine | −32 | foam | 379.2130 |

1% solution in MeOH

### Example 154

### Preparation of (R)-6-(1H-benzo[d]imidazol-1-yl)-2-(2-(2-methylpyrrolidin-1-yl)ethyl)-3,4-dihydroisoquinolin-1(2H)-one hydrochloride

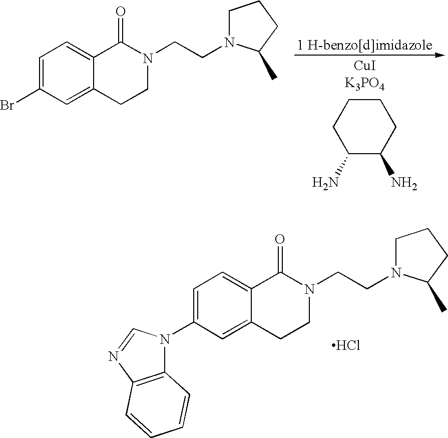

Using essentially the same procedure described in Example 35 and startying from benzoimidazole (0.11 g, 0.88 mmol) and 6-iodo-2-(2-(pyrrolidin-1-yl)ethyl)-3,4-dihydroisoquinolin-1(2H)-one (0.17 g, 0.44 mmol), the desired compound was obtained as a white foam, [α]_{D}²⁵=−32° (c=1.00% in methanol), HRMS (ES) m/z 375.2186 [M+H]⁺.

### Example 155-156

### Preparation of N-substituted-1-oxo-2-(2-(pyrrolidin-1-yl)ethyl)-1,2,3,4-tetrahydroiso-quinoline-6-carboxamide hydrochloride compounds

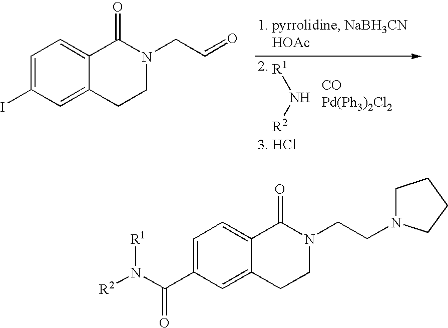

### Step 1: 6-Iodo-2-(2-(Pyrrolidin-1-yl)ethyl)-3,4-dihydroisoquinolin-1(2H)-one

Using essentially the same procedure described in step 4 of Example 143 and employing pyrrolidine (1.98 g, 6.3 mmol), the title compound 1.0 g (90%) was obtained as a white foam, MS (ES) m/z 371.0 [M+H]⁺.

### Step 2: N-Substituted-1-oxo-2-(2-(pyrrolidin-1-yl)ethyl)-1,2,3,4-tetrahydroiso-quinoline-6-carboxamide hydrochlorides

Using essentially the same procedure described in step 2 Example 38 and employing 6-iodo-2-(2-(pyrrolidin-1-yl)ethyl)-3,4-dihydroisoquinolin-1(2H)-one (0.1g, 0.24 mmol), the compounds shown in Table XIV were obtained and identified by NMR and high resolution mass spectral analyses.

TABLE XIV

| Ex. No. | NHR¹R² | mp ° C. | [M + H] |
|---|---|---|---|
| 155 | piperidine | 144-147 | 356.2334 |
| 156 | isoindoline | 220-221 | 390.2181 |

### Example 157

### Preparation of (R)-2-(2-(2-methylpyrrolidin-1-yl)ethyl)-3,3′,4,4′-tetrahydro-6,6′-biisoquinoline-1,1′(2H,2′H-dione hydrochloride

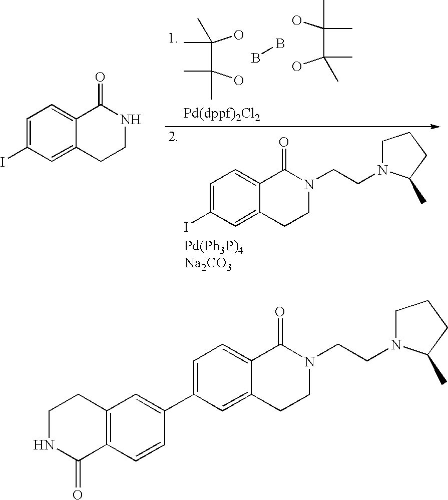

### Step 1: 6-(4,4,5,5-Tetramethyl-1,3,2-dioxaborolan-2-yl)-3,4-dihydroisoquinolin-1(2H)-one

A solution of 6-bromo-3,4-dihydroisoquinolin-1(2H)-one (25 g, 111 mol) in dioxane (750 mL) was degassed for 30 min. Potassium acetate (43.41 g, 442.3 mol) and bis(pinacolato)diborane (43 g, 169.2 mol) were added and degassed again for 30 min. Then PdCl₂(dppf)₂ (4.5 g, 5.5 mol) was added, degassed at 60° C. for 10 min and then the reaction mixture was heated at 90° C. overnight. The reaction mixture was filtered, washed with ethyl acetate, and the combined solvent was concentrated in vacuo. The residue was re-dissolved in ethyl acetate, washed with water followed by brine, dried over sodium sulfate and concentrated in vacuo. The crude product was purified by column chromatography (Silica, 10-50% ethyl acetate in hexane) and followed by recrystalization from ethyl acetate to provide 16 g (53%) of title compound as a white solid, mp 234.4-236.9° C.; MS (ES) m/z 274.1 [M+H]⁺.

### Step 2: (R)-2-(2-(2-methylpyrrolidin-1-yl)ethyl)-3,3′,4,4′-tetrahydro-6,6′-biisoquinoline-1,1′-(2H,2′H)-dione

Using essentially the same procedure described in Example 9 and employing 6-(4,4,5,5-tetramethyl-1,3,2-dioxaborolan-2-yl)-3,4-dihydroisoquinolin-1(2H)-one (81 mg, 0.3 mmol), (R)-6-iodo-2-(2-(2-methylpyrrolidin-1-yl)ethyl)-3,4-dihydroisoquinolin-1(2H)-one (0.06 g, 0.15 tetrakis(triphenylphosphine)palladium (9.0 mg) and sodium carbonate (41 mg, 0.38 mmol), the title product 0.14 g (32%) was obtained as a white solid, mp 272-273.5° C.; [α]_{D}²⁵=−27° (c=1.00% in methanol), HRMS (ES) m/z 404.2336 [M+H]⁺.

### Example 158

### Preparation of 2-methyl-2′-(2-(pyrrolidin-1-yl)ethyl)-3,3′,4,4′-tetrahydro-6,6′-biisoquinoline-1,1′(2H,2′H)-dione hydrochloride

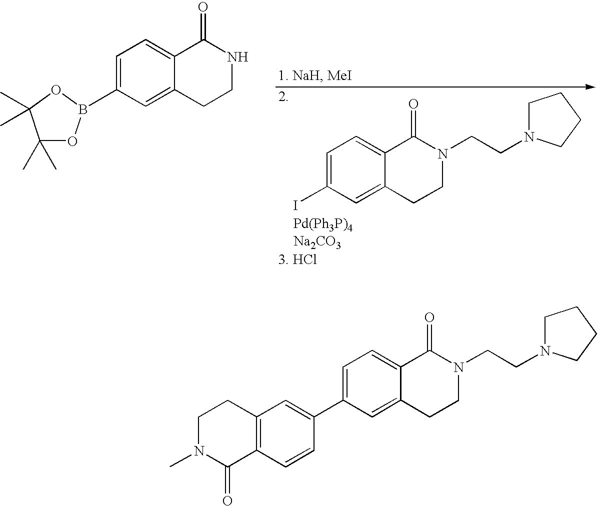

### Step 1: 2-Methyl-6-(4,4,5,5-tetramethyl-1,3,2-dioxaborolan-2-yl)-3,4-dihydroiso-quinolin-1(2H)-one

A suspension of sodium hydride (60% dispersion in mineral oil, 0.17 g, 4.4 mmol) in N,N-dimethylformamide at 0° C., under nitrogen, was treated dropwise over 15 min with a solution of 6-(4,4,5,5-tetramethyl-1,3,2-dioxaborolan-2-yl)-3,4-dihydroisoquinolin-1(2H)-one (0.5 g, 2.2 mmol in N,N-dimethylformamide, stirred at 0° C. for an additional 20 min, treated with methyl iodide (0.29 mL, 3.3 mmol) at 0° C., allowed to warm to room temperature, stirred for 4 h and was quenched with water (1 mL) and the solvent was removed in vacuo to afford the desired product that was used in next step without purification.

### Step 2: 2-methyl-2′-(2-(pyrrolidin-1-yl)ethyl)-3,3′,4,4′-tetrahydro-6,6′-biisoquinoline-1,1′(2H,2′H)-dione

Using essentially the same procedure described in Example 9 and employing 6-iodo-2-(2-(pyrrolidin-1-yl)ethyl)-3,4-dihydroisoquinolin-1(2H)-one (42 mg, 0.11 mmol), the desired compound 16 mg (35%) was obtained as a white solid, mp 225-226° C., HRMS (ES) m/z 404.2350 [M+H]⁺.

### Example 159

### Preparatio of (R)-2-methyl-2′-(2-(2-methylpyrrolidin-1-yl)ethyl)-3,3′,4,4′-tetrahydro-6,6′-biisoquinoline-1,1′(2H,2′H-dione hydrochloride

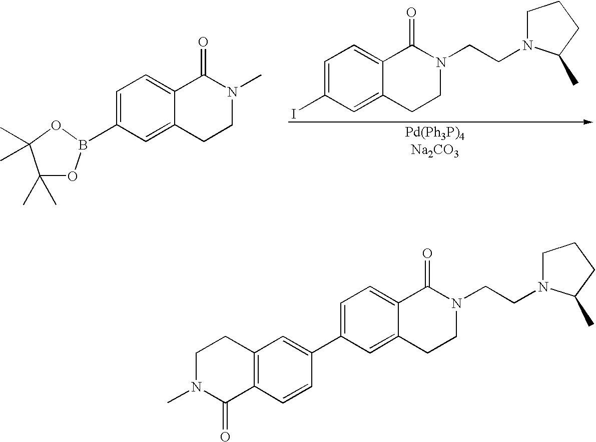

Using essentially the same procedure described in Example 158 and employuing (R)-6-iodo-2-(2-(2-methylpyrrolidin-1-yl)ethyl)-3,4-dihydroisoquinolin-1(2H)-one (40 mg, 0.1 mmol), the desired compound was obtained as a white solid; mp >270° C., HRMS (ES) m/z 418.2493 [M+H]⁺.

### Example 160

### Preparation of 2-{2-[(2R)-2-methylpyrrolidin-1-yl]ethyl}-6-[4-(pyrrolidin-1-ylcarbonyl)phenoxy]-3,4-dihydroisoquinolin-1(2H)-one hydrochloride

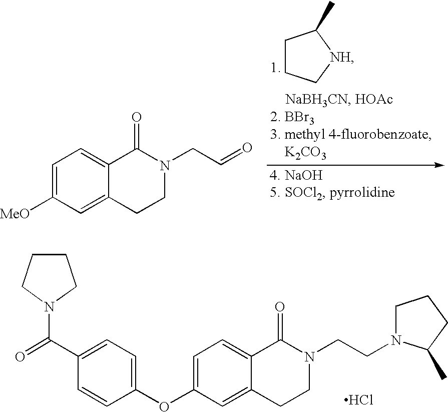

### Step 1: (R)-6-Methoxy-2-(2-(2-methylpyrrolidin-1-yl)ethyl)-3,4-dihydro-isoquinolin-1(2H)-one

Using essentially the same procedure described in Example 87 (step 3) and employing (R)-2-methyl pyrrolidine, (R)-6-methoxy-2-(2-(2-methylpyrrolidin-1-yl)ethyl)-3,4-dihydroisoquinolin-1(2H)-one was obtaianed as a white foam, [α]_{D}²⁵=−66° (1% solution in methanol), MS (ES) 289.1 [M+H]⁺.

### Step 2: (R)-6-Hydroxy-2-(2-(2-methylpyrrolidin-1-yl)ethyl)-3,4-dihydro-isoquinolin-1(2H)-one

Using essentially the same procedure described in Example 88 and employing (R)-6-methoxy-2-(2-(2-methylpyrrolidin-1-yl)ethyl)-3,4-dihydroisoquinolin-1(2H)-onemp, (R)-6-hydroxy-2-(2-(2-methylpyrrolidin-1-yl)ethyl)-3,4-dihydroisoqui-nolin-1(2H)-one was obtained as light yellow oil, [α]_{D}²⁵=−22° (1% solution in methanol), MS (ES) m/z 275.2 [M+H]⁺.

### Step 3: (R)-Methyl 4-(2-(2-(2-methylpyrrolidin-1-yl)ethyl)-1-oxo-1 2,3,4-tetrahydroisoquinolin-6-yloxy)benzoate

Using essentially the same procedure described in Example 89 and employing (R)-6-hydroxy-2-(2-(2-methylpyrrolidin-1-yl)ethyl)-3,4-dihydroisoquinolin-1(2H)-one, (R)-methyl 4-(2-(2-(2-methylpyrrolidin-1-yl)ethyl)-1-oxo-1,2,3,4-tetrahydro-isoquinolin-6-yloxy)benzoate was obtained as a colorless oil, [α]_{D}²⁵=−42° (1% solution in methanol), HRMS (ES) m/z 409.2126 [M+H]⁺.

### Step 4: (R)-4-(2-(2-(2-Methylpyrrolidin-1-yl)ethyl)-1-oxo-1,2,3,4-tetrahydro-isoquinolin-6-yloxy)benzoic acid

Using essentially the same procedure described in Example 90 and employing (R)-methyl 4-(2-(2-(2-methylpyrrolidin-1-yl)ethyl)-1-oxo-1,2,3,4-tetrahydro-isoquinolin-6-yloxy)benzoate, (R)-4-(2-(2-(2-methylpyrrolidin-1-yl)ethyl)-1-oxo-1,2,3,4-tetrahydro-isoquinolin-6-yloxy)benzoic acid was prepared as white foam, (ES) m/z 395.2 [M+H]⁺.

### Step 5: 2-{2-[(2R)-2-Methylpyrrolidin-1-yl]ethyl}-6-[4-(pyrrolidin-1-ylcarbonyl)-phenoxy]-3,4-dihydroisoquinolin-1(2H)-one

Using essentially the same procedure described in Example 55 and employing (R)-4-(2-(2-(2-methylpyrrolidin-1-yl)ethyl)-1-oxo-1,2,3,4-tetrahydroisoqui-nolin-6-yloxy)benzoic acid and pyrroldine, 2-{2-[(2R)-2-methylpyrrolidin-1-yl]ethyl}-6-[4-(pyrrolidin-1-ylcarbonyl)phenoxy]-3,4-dihydroisoquinolin-1(2H)-one was obtained as a white foam, [α]_{D}²⁵=−27° (1% solution in methanol), (ES) m/z 448.2 [M+H]⁺.

### Example 161-164

### Preparation of 2-(2-(substitutedamino)ethyl)-6-piperidin-1-yl-3,4-dihydroiso-quinolin-1(2H)-one hydrochloride compounds

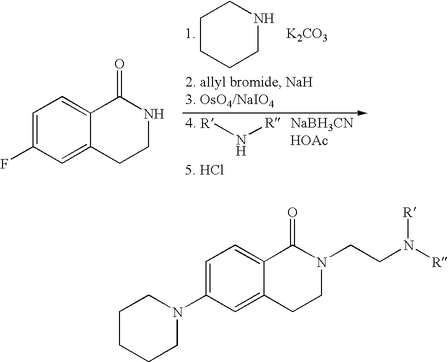

### Step 1: 6-(Piperidin-1-yl)-3,4-dihydroisoquinolin-1(2H)-one

A mixture of 6-fluoro-3,4-dihydroisoquinolin-1(2H)-one (1.0 g, 60 mmol), potassium carbonate (2.1 g, 15 mmol) and piperidine (3.0 mL, 30 mmol) in DMSO was stirred at 120° C. overnight, diluted with water and extracted with methylene chloride. The combined extracts were washed with water, dried over sodium sulfate and concentrated in vacuo. The residue was purified by ISCO CombiFlash® chromatography (silica, 0-10% methanol in methylene chloride) to afford the title compound 1.18 g (84%) as a yellow oil; HRMS (ES) m/z 231.1492 [M+H]⁺.

### Step 2: 2-Allyl-6-(piperidin-1-yl)-3,4-dihydroisoquinolin-1(2H)-one

According to the procedure described for 3a, starting from 6-(piperidin-1-yl)-3,4-dihydroisoquinolin-1(2H)-one (1.18 g, 5.1 mmol), 0.8 g (58%) of title compound was obtained as a light yellow oil. HRMS (ES) m/z 271.1806 [M+H]⁺.

### Step 3: 2-(1-Oxo-6-(piperidin-1-yl)-3,4-dihydroisoquinolin-2(1H)-yl)acetaldehyde

According to the procedure described for 6a, starting from 2-allyl-6-(piperidin-1-yl)-3,4-dihydroisoquinolin-1(2H)-one (0.7 g, 2.6 mmol), 0.7 g (100%) of the title product was obtained as a yellow oil. [α]_{D}²⁵=−3° (1% solution in methanol), HRMS (ES) m/z 273.1597 [M+H]⁺.

### Step 4: 2-(2-(Ethylamino)ethyl)-6-(piperidin-1-yl)-3,4-dihydroisoquinolin-1(2H)-one hydrochlorides

Using essentially the same procedure described in Example 7 and employing 2-(1-oxo-6-(piperidin-1-yl)-3,4-dihydroisoquinolin-2(1H)-yl)acetaldehyde (60 mg, 0.22 mmol) and the desired amines as the starting material, the compounds shown in Table XIV were obtained and identified by NMR and high resolution mass spectral analyses.

TABLE XIV

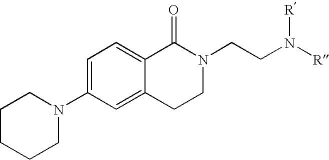

| Ex. No. | NHR¹R² | [α]_{D}²⁵* | mp ° C. | [M + H] |
|---|---|---|---|---|
| 161 | (R)-2-methyl pyrrolidine | −25 | >225° C. | 342.2531 |
| 162 | pyrrolidine | — | 245-246 | 328.2382 |
| 163 | piperidine | — | 245-246 | 342.2541 |
| 164 | azepan | — | 229-231 | 356.2696 |

c = 1% SOLUTION, MeOH

### Example 165-166

### Preparation of (R)-6-(substituted amino)-2-(2-(2-methylpyrrolidin-1-yl)ethyl)-3,4-dihydroiso-quinolin-1(2H)-one

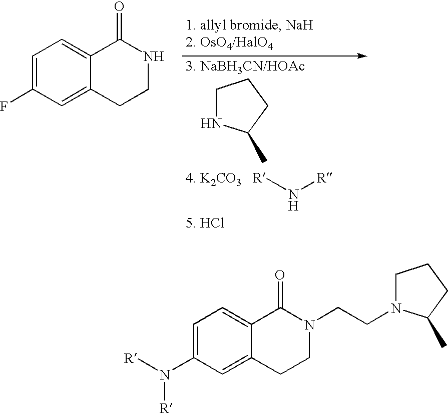

### Step 1: 2-Allyl-6-fluoro-3,4-dihydroisoquinolin-1(2H)-one

According to the procedure described for 3a, starting from 6-fluoro-3,4-dihydroisoquinolin-1(2H)-one (3.3 g, 20 mmol), 3.5 g (85%) of title compound was obtained as a light yellow oil. HRMS (ES) m/z 206.0974 [M+H]⁺.

### Step 2: 2-(6-Fluoro-1-oxo-3,4-dihydroisoquinolin-2(1H)-yl)acetaldehyde

According to the procedure described for 6a, starting from 2-allyl-6-fluoro-3,4-dihydroisoquinolin-1(2H)-one (3.5 g, 217 mmol), 2.75 g (73%) of the title product was obtained as a light yellow oil. MS (ES) m/z 208.0 [M+H]⁺.

### Step 3: (R)-6-Fluoro-2-(2-(2-methylpyrrolidin-1-yl)ethyl)-3,4-dihydroisoquinolin-1(2H)-one

Using essentially the same procedure described in Example 7 and employing 2-(6-fluoro-1-oxo-3,4-dihydroisoquinolin-2(1H)-yl)acetaldehyde (1.85 g, 8.4 mmol), ), 2.0 g (86%) of the title product was obtained as a white foam. [α]_{D}²⁵=−37° (1% solution in methanol), MS (ES) m/z 277.1 [M+H]⁺.

### Step 4: (R)-6-(Substituted amino)-2-(2-(2-methylpyrrolidin-1-yl)ethyl)-3,4-dihydroisoquinolin-1(2H)-one hydrochlorides

Using essentially the same procedure described in Example and employing (R)-6-fluoro-2-(2-(2-methylpyrrolidin-1-yl)ethyl)-3,4-dihydroisoquinolin-1(2H)-one (0.15 g, 0.5 mmol) and the desired amines as the starting material, the compounds shown in Table XV were obtained and identified by NMR and high resolution mass spectral analyses.

TABLE XV

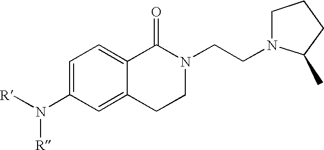

| Ex. No. | NHR¹R² | [α]_{D}²⁵* | mp ° C. | [M + H] |
|---|---|---|---|---|
| 165 | pyrrolidine | −32 | 182-183 | 328.2384 |
| 166 | azepan | — | 182-184 | 356.2699 |

c = 1% SOLUTION, MeOH

### Example 167-170

### Preparation of (R)-3-fluoro-N-substituted-4-(2-(2-(2-methylpyrrolidin-1-yl)ethyl)-1-oxo-1,2,3,4-tetrahydroisoquinolin-6-yl)benzamide hydrochloride compounds

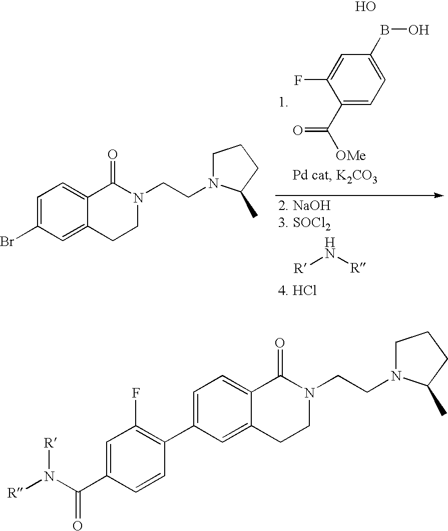

### Step 1: (R)-Methyl 3-fluoro-4-(2-(2-(2-methylpyrrolidin-1-yl)ethyl)-1-oxo-1,2,3,4-tetrahydroisoquinolin-6-yl)benzoate

Using essentially the same procedure described in Example 9 and employing (R)-6-bromo-2-(2-(2-methylpyrrolidin-1-yl)ethyl)-3,4-dihydroisoquinolin-1(2H)-one (0.39 g, 1.2 mmol) and 2-fluoro-4-(methoxycarbonyl)phenylboronic acid, the title product 0.38 g (82%) was obtained as a light yellow oil, [α]_{D}²⁵=−44° (1% solution in methanol), HRMS (ES) m/z 411.2074 [M+H]⁺.

### Step 2: (R)-3-Fluoro-4-(2-(2-(2-methylpyrrolidin-1-yl)ethyl)-1-oxo-1,2,3,4-tetrahydroisoquinolin-6-yl)benzoic acid

Using essentially the same procedure described in Example 53 and employing (R)-methyl 3-fluoro-4-(2-(2-(2-methylpyrrolidin-1-yl)ethyl)-1-oxo-1,2,3,4-tetrahydroiso-quinolin-6-yl)benzoate (0.36 g, 0.88 mmol), the title compound 0.27 g (80%) was obtained as a white foam, [α]_{D}²⁵=−16° (1% solution in methanol), MS (ES) m/z 396.1 [M+H]⁺.

### Step 3: (R)-3-Fluoro-N-substituted-4-(2-(2-(2-methylpyrrolidin-1-yl)ethyl)-1-oxo-1,2,3,4-tetrahydroisoquinolin-6-yl)benzamide

Using essentially the same procedure described in Example 55 and employing (R)-3-fluoro-4-(2-(2-(2-methylpyrrolidin-1-yl)ethyl)-1-oxo-1,2,3,4-tetrahydroiso-quinolin-6-yl)benzoic acid and the desired amine, the compounds shown in Table XVI were obtained and identified by NMR and mass spectral analyses.

TABLE XVI

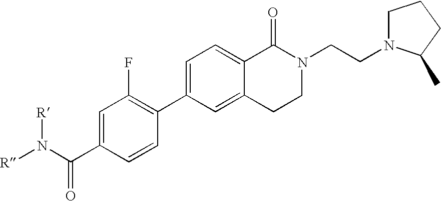

| Ex. No. | R″R′NH | [α]_{D}²⁵* | mp ° C. | [M + H] |
|---|---|---|---|---|
| 167 | NHMe₂ | −26 | 219-220 | 425.2 |
| 168 | pyrrolidine | −28 | 213-215 | 450.2 |
| 169 | NHMe | −36 | 231-232 | 410.2 |
| 170 | NHEt | −28 | 209-210 | 424.2 |

c = 1% SOLUTION, MeOH

### Example 171-178

### Preparation (R)-2-(2-(pyrrolidin-1-yl)ethyl)-6-arylsubstituted-3,4-dihydro-isoquinolin-1(2H)-one

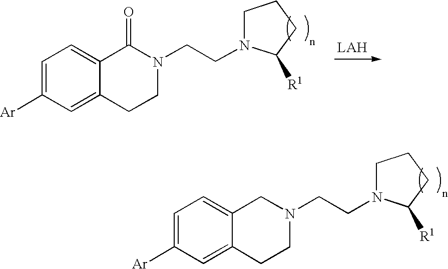

To a solution of (R)-2-(2-(pyrrolidin-1-yl)ethyl)-6-arylsubstituted-3,4-dihydro-isoquinolin-1(2H)-one (1.0 eq) in dry tetrahydrofuran (10 mL) at −10° C. was added lithium aluminum hydride (2.0 M solution in THF, 2.0 eq). The reaction mixture was heated at 78° C. for 30 min, cooled to room temperature and quenched with water. The reaction mixture was extracted with methylene chloride. The combined extracts were washed with water, dried over sodium sulfate and concentrated in vacuo. The residue was purified by Gilson Prep-HPLC (5-70% acetontrile in water with 0.3% TFA). The compounds shown in Table XIV were obtained and identified by NMR and high resolution mass spectral analyses.

TABLE XIV

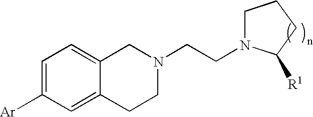

| Ex. | | | | | | |
|---|---|---|---|---|---|---|
| No. | Ar | R¹ | n | mp ° C. | [M + H] | [α]_{D}²⁵* |
| 171 | 4-trifluoro- | H | 1 | >230 | 391.1993 | — |
| | methoxy phenyl | | | | | |
| 172 | 4-fluorophenyl | (R)—Me | 1 | 232-234 | 339.2234 | −26 |
| 173 | 3-fluorophenyl | H | 1 | 272-274 | 325.2078 | — |
| 174 | 4-fluorophenyl | H | 2 | 272-274 | 339.2237 | — |
| 175 | 4-fluorophenyl | H | 3 | 258-260 | 353.2393 | — |
| 176 | 5-benzodioxole | H | 1 | 257-259 | 351.2067 | — |
| 177 | 4-fluorophenyl | H | 1 | 250-251 | 325.2070 | — |
| 178 | phenyl | H | 1 | 268-270 | 307.2163 | — |

c = 1% SOLUTION, MeOH—

### Example 179

### Preparation of (R)-5-(4-fluorophenyl)-2-(2-(2-methylpyrrolidin-1-yl)ethyl)-isoindoline hydrochloride

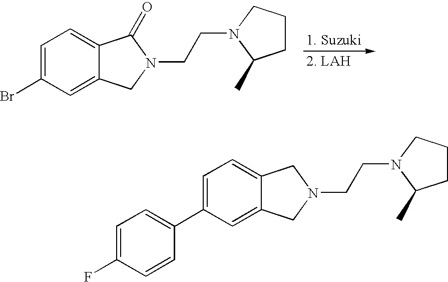

### Step 1: (R)-5-(4-Fluorophenyl)-2-(2-(2-methylpyrrolidin-1-yl)ethyl)isoindolin-1-one

Using essentially the same procedure described in Example 9 and employing (R)-5-bromo-2-(2-(2-methylpyrrolidin-1-yl)ethyl)isoindolin-1-one (0.07 g, 0.18 mmol), the title compound 62 (85%) was obtained as a yellow oil.

### Step 2: (R)-5-(4-fluorophenyl)-2-(2-(2-methylpyrrolidin-1-yl)ethyl)-isoindoline hydrochloride

Using essentially the same procedure described in Example 178 and employing ((R)-5-(4-fluorophenyl)-2-(2-(2-methylpyrrolidin-1-yl)ethyl)isoindolin-1-one (62 mg, 0.18 mmol), the title compound 55 mg (88%) was obtained as a white solid, mp 250-251° C.; HRMS (ES) m/z 325.2074 [M+H]⁺.

### Example 180-187

### Preparation of N-substitted 4-(2-(2-(pyrrolidin-1-yl)ethyl)-1,2,3,4-tetrahydro-isoquinolin-6-yl)benzamide hydrochlorides

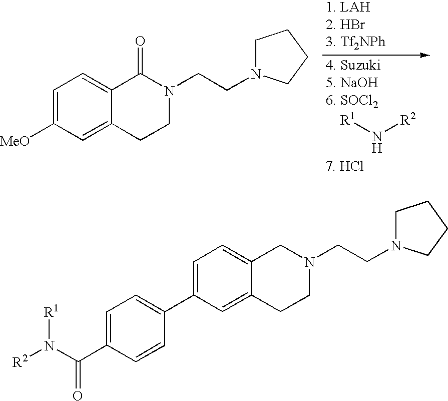

### Step 1: 6-Methoxy-2-(2-(pyrrolidin-1-yl)ethyl)-1,2,3,4-tetrahydroisoquinoline

To a solution of 6-methoxy-2-(2-(pyrrolidin-1-yl)ethyl)-3,4-dihydroisoquinolin-1(2H)-one (13 g, 47 mmol) in THF (500 mL) at 0° C. was added lithium aluminum hydride (2.0 M in THF, 142 mL, 282 mmol) and the reaction mixture was allowed to stir at room temperature for 3 hours under nitrogen atmosphere. The reaction mixture was allowed to cool to room temperature and quenched with aqueous sodium hydroxide, followed by water and stirred for 2 h. The suspension was filtered through a pad of celite pad and the filtrate was evaporated under reduced pressure to afford 81.3% of the title compound as a colorless oil that was taken for next step without further purification.

### Step 2: 2-(2-(Pyrrolidin-1-yl)ethyl)-1,2,3,4-tetrahydroisoquinolin-6-ol

A mixture of 6-methoxy-2-(2-(pyrrolidin-1-yl)ethyl)-1,2,3,4-tetrahydroisoquinoline (14 g, 54 mmol) and hydrobromic acid (47% aq, 17.2 g) was stirred at 80° C. for 12 hours. Excess hydrobromic acid was evaporated under vacuo and the residue was dissolved in methanal and partially solvent evaporated under reduced pressure. Precipitated salt was filtered, and washed with chilled methanol. This salt was partitioned between 2% K₂CO₃ and ethyl methyl ketone, organic layer separated, washed with brine, dried over sodium sulfate and solvent concentrated under reduced pressure to afford the title compound as a brown color solid, Yield: 76.5%; %); HRMS (ES) m/z 247.1804 [M+H]⁺.

### Step 3: 2-(2-(Pyrrolidin-1-yl)ethyl)-1,2,3,4-tetrahydroisoquinolin-6-yl trifluoromethane-sulfonate

A mixture of on 2-(2-(pyrrolidin-1-yl)ethyl)-1,2,3,4-tetrahydroisoquinolin-6-ol (3.3 g, 13 mmol), N-phenyltrifluoromethanesulfonimide (7.2 g, 19.5 mmol) and trimethylamine (2.8 mL, 19.5 mmol) in methylene chloride was stirred at room temperature overnight, diluted with water and extracted with methylene chloride. The combined extracts were washed with water, dried over sodium sulfate and concentrated in vacuo. The residue was purified by ISCO CombiFlash® chromatography (silica, 0-10% methanol in methylene chloride) to afford the title compound as a yellow oil, 4.6 g (90%); HRMS (ES) m/z 379.1304 [M+H]⁺.

### Step 4: Methyl 4-(2-(2-(pyrrolidin-1-yl)ethyl)-1 2,3,4-tetrahydroisoquinolin-6-yl)benzoate

Using essentially the same procedure described in Example 9, starting from 2-(2-(pyrrolidin-1-yl)ethyl)-1,2,3,4-tetrahydroisoquinolin-6-yl trifluoromethane-sulfonate (4.3 g, 0.91 mol) and 4-methoxycarbonyl phenyl boronic acid (8.1 g, 3.6 mol), 2.4 g (59%) of the title product was obtained as a colorless oil. The hydrochloric salt was prepared in ethanol and collected as a white solid, mp 266-267° C., HRMS (ES) m/z 365.2226 [M+H]⁺.

### Step 5: 4-(2-(2-(Pyrrolidin-1-yl)ethyl)-1,2,3,4-tetrahydroisoquinolin-6-yl)benzoic acid

Using essentially the same procedure described in Example 53 and employing methyl 4-(2-(2-(pyrrolidin-1-yl)ethyl)-1,2,3,4-tetrahydroisoquinolin-6-yl)benzoate (2.42 g, 6.6 mmol) as the starting material, the title compound 2.1 g (90%) was prepared as white solid, mp 269-271° C., HRMS (ES) m/z 351.2064 [M+H]⁺.

### Step 6: N,N-dimethyl-4-(2-(2-(pyrrolidin-1-yl)ethyl)-1,2,3,4-tetrahydroiso-quinolin-6-yl)benzamide

Using essentially the same procedure described in Example 55 and employing 4-(2-(2-(pyrrolidin-1-yl)ethyl)-1,2,3,4-tetrahydroisoquinolin-6-yl)benzoic acid and the desired amines as the starting materials, the compounds shown in Table XVII were obtained and identified by NMR and high resolution mass spectral analyses.

TABLE XVII

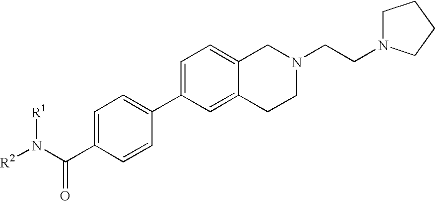

| EX. No. | NR¹R² | mp ° C. | [M + H] |
|---|---|---|---|
| 180 | NH2 | 262-264 | 350.2229 |
| 181 | NHMe | 280-281 | 364.2388 |
| 182 | NHEt | 266-268 | 378.254 |
| 183 | NMe2 | 248-250 | 378.2542 |
| 184 | pyrrolidne | 241-242 | 404.2692 |
| 185 | piperidine | 270-272 | 418.2856 |
| 186 | morpholine | 260-261 | 420.2649 |
| 187 | (R)-2-Me-pyrrolidine | foam | 418.2856 |

### Example 188-190

### Preparation of (R)-substituted-4-(2-(2-(2-methylpyrrolidin-1-yl)ethyl)-1,2,3,4-tetrahydroiso-quinolin-6-yl)benzamide hydrochloride compounds

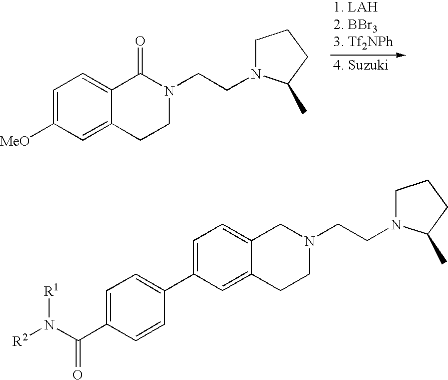

### Step 1: (R)-6-methoxy-2-(2-(2-methylpyrrolidin-1-yl)ethyl)-1,2,3,4-tetrahydroiso-quinoline

Using essentially the same procedure described in step 1 of Example 180 and employing (R)-6-methoxy-2-(2-(2-methylpyrrolidin-1-yl)ethyl)-3,4-dihydroisoquinolin-1(2H)-one (1.05 g, 3.6 mol), 0.75 g (75%) of the title product was obtained as a colorless oil. HRMS (ES) m/z 275.2120 [M+H]⁺.

### Step 2: (R)-2-(2-(2-methylpyrrolidin-1-yl)ethyl)-1,2,3,4-tetrahydroisoquinolin-6-ol

Using essentially the same procedure described in Example 43 and employing (R)-6-methoxy-2-(2-(2-methylpyrrolidin-1-yl)ethyl)-3,4-dihydroisoquinolin-1(2H)-one (0.75 g, 2.7 mol), 0.55 g (78%) of the title product was obtained as an off-white foam. [α]_{D}²⁵=11 (1% solution in methanol); HRMS (ES) m/z 261.1960 [M+H]⁺.

### Step 3: (R)-2-(2-(2-methylpyrrolidin-1-yl)ethyl)-1,2,3,4-tetrahydroisoquinolin-6-yl trifluoromethanesulfonate

Using essentially the same procedure described in step 3 of Example 180 and employing (R)-2-(2-(2-methylpyrrolidin-1-yl)ethyl)-1,2,3,4-tetrahydroisoquinolin-6-ol (0.55 g, 2.1 mol), 0. g (%) of the title product was obtained as an off-white foam. [α]_{D}²⁵=−68° (1% solution in methanol); HRMS (ES) m/z 393.1455 [M+H]⁺.

### Step 4: (R)-N-substituted-4-(2-(2-(2-methylpyrrolidin-1-yl)ethyl)-1,2,3,4-tetrahydroiso-quinolin-6-yl)benzamide

Using essentially the same procedure described in step 4 of Example 180 and employing (R)-2-(2-(2-methylpyrrolidin-1-yl)ethyl)-1,2,3,4-tetrahydroisoquinolin-6-yl trifluoromethanesulfonate and the desired phenyl boronic acids, the compounds shown in Table XVIII were obtained and identified by NMR and high resolution mass spectral analyses.

TABLE XVIII

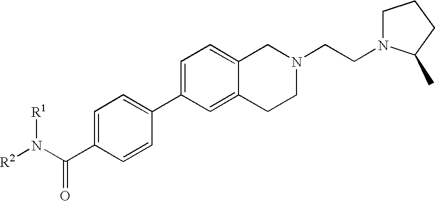

| EX. No. | NR¹R² | mp ° C. | [M + H] |
|---|---|---|---|
| 188 | HNMe | >180 | 378.2532 |
| 189 | HNEt | 178-180 | 392.2695 |
| 190 | morpholine | 138-140 | 434.2798 |

### Example 191-200

### Preparation of (R)-N-substituted-4-(2-(2-(2-methylpyrrolidin-1-yl)ethyl)-1-oxo-1,2,3,4-tetrahydroisoquinolin-6-yl)benzamide hydrochloride compounds

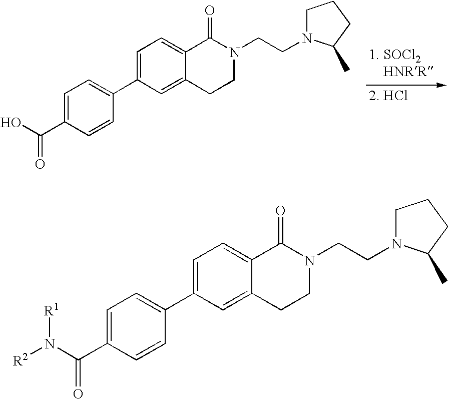

Using essentially the same procedure described in Example 55 and employing (R)-4-(2-(2-(2-methylpyrrolidin-1-yl)ethyl)-1-oxo-1,2,3,4-tetrahydroisoquinolin-6-yl)-benzoic acid and the desired amine, the compounds shown in Table XIX were obtained and identified by NMR and mass spectral analyses.

TABLE XIX

| Ex. | | Ex. | |
|---|---|---|---|
| No. | HNR¹R² | No. | HNR′R″ |
| 191 | HNMe2 | 192 | azetidine |
| 193 | 2-methoxyethanamine | 194 | 2-fluoroethanamine |
| 195 | morpholine | 196 | piperidine |
| 197 | (S)-1-methoxypropan-2- | 198 | (S)-2- |
| | amine | | (methoxymethyl)pyrrolidine |
| 199 | 2-isopropoxyethanamine | 200 | HNMeEt |

### Evaluation of Methyl Histamine Binding in Human Histamine H₃ Receptor Cell Line

The affinity of test compounds for the histamine 3 (H₃) receptor is evaluated in the following manner. Stably transfected HEK293T cells are grown in DMEM containing 10% heat inactivated FBS and G-418 (500 ug/ml). Cells are scraped from the plate, transferred to centrifuge tubes, washed one time in PBS by centrifugation in a Sorvall RT7 Plus centrifuge (2000 rpm 10 minutes, 4° C.). The resulting pellets are stored at −80° C. until ready for use. Cells are re-suspended in buffer (50 mM Tris pH=7.5) and placed in a Dounce homogenizer, douncing ten times to homogenize cells. The homogenate is spun down by centrifugation (Sorvall RT7 Plus, 1800 rpm 10 minutes, 4° C.). The supernatant is placed in a Corex tube and spun down by centrifugation (Sorvall RC 5c Plus, 17,000 rpm 20 minutes, 4° C.). The pellet is resuspended in buffer (50 mM Tris, pH 7.5). Protein concentration (ug/ul) is determined using the Micro-BCA Protein Determination. The binding assay is set up in a 96 well microtiter plate in a total volume of 250 uL. Non-specific binding is determined in the presence of 10 uM clobenpropit. The final radioligand concentration is 1 nM. The test compound is serially diluted using the Beckman Biomek2000 to a final approximate range of 100 uM to 100 pM. Membranes are suspended in buffer, homogenized in 2 bursts of ten seconds using a Vitris mechanical homogenizer set at power setting 5. Ten μg of membranes are added to each well. Following a one hour incubation at 30° C., the reaction is terminated by the addition of ice cold buffer and rapid filtration with a Packard Filtermate Harvester through a GF/B filter pre-soaked with 1% PEI for one hour. The plate is dried for one hour at 37° C. and 60 μL Microscint Scintillant is added to each well. The CPM per well is measured on a Packard Top Count NXT. Ki values are determined in nM. The Ki is calculated from the IC₅₀ (i.e. the concentration of competing ligand which displaces 50% of the specific binding of the radioligand). CPM values are expressed as % specific binding and plotted vs compound concentration. A curve is fitted using a four-parameter logistic fit and the IC₅₀ value is determined. The Ki is calculated from this using the Cheng-Prusoff equation: pKi=IC₅₀/1+(L/Kd) where L=concentration of free radioligand used in the assay, and Kd is the dissociation constant of the radioligand for the receptor. L is determined for each experiment by counting an aliquot of the diluted radioligand (corresponding to that added to each well) and the Kd has previously been determined under identical conditions for this cell line/radioligand.

### Cyclic AMP Assay for Histamine Receptor H₃ Antagonism Activity.

Stable H₃ cells are maintained in tissue culture flask in DMEM with high glucose, 10% FBS, 1× pen/strep, 500 ug/ml GY18, until experiment. Culture media is removed and cells are washed twice with PBS w/Ca++ and Mg++ plus 500 μM IBMX. Cells are then detached by tapping on the side of the flask and resuspend in the same buffer. Two thousand cells/well are incubated with 1 μM histamine plus 10 μM forskolin plus various concentrations of compounds in a total volume of 30 μL in 96 well plates for 30 min at 30° C. Final test compound concentrations range from 1 0-4M to 1 0-9.5M at full log dilutions. Cyclic AMP levels are measured using HitHunter cAMP kit from Discoverx, cat# 900041 according to manufacturer's instruction. Chemiluminescence signals are detected using Top Count (Packard).

Cyclic AMP levels in control cells receiving 10 pM forskolin plus 100 nM histamine are considered 0%, and in cells receiving 10 uM forskolin plus 100 nM histamine plus 1 μM clobenpropit are considered 100%. Data are expressed as % control and analyzed using Prizm soft ware. The Kb values are calculated using the following equation, KB=EC₅₀ or IC₅₀/[1+(ligand/Kd)]. The data are shown in Table XV, below.

TABLE XVI

| Ex | H₃ |
|---|---|
| No | Binding Ki (nM) |
| 9 | B |
| 10 | A |
| 11 | A |
| 12 | B |
| 13 | C |
| 14 | B |
| 15 | A |
| 16 | B |
| 17 | A |
| 18 | B |
| 19 | A |
| 20 | A |
| 21 | B |
| 22 | A |
| 23 | A |
| 25 | A |
| 26 | A |
| 27 | D |
| 28 | B |
| 32 | A |
| 33 | C |
| 34 | A |
| 35 | A |
| 36 | A |
| 37 | B |
| 38 | B |
| 39 | B |
| 40 | A |
| 41 | A |
| 42 | A |
| 43 | A |
| 44 | A |
| 45 | B |
| 47 | B |
| 48 | A |
| 49 | A |
| 50 | A |
| 51 | A |
| 52 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | D |
| 72 | B |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 91 | B |
| 92 | B |
| 93 | A |
| 94 | A |
| 95 | C |
| 96 | C |
| 97 | B |
| 98 | A |
| 99 | A |
| 101 | C |
| 102 | A |
| 103 | A |
| 108 | A |
| 112 | C |
| 117 | B |
| 118 | D |
| 119 | E |
| 120 | A |
| 121 | A |
| 122 | A |
| 123 | A |
| 124 | A |
| 125 | A |
| 126 | A |
| 127 | A |
| 128 | A |
| 128 | A |
| 130 | A |
| 131 | A |
| 132 | A |
| 133 | A |
| 134 | A |
| 135 | A |
| 137 | A |
| 138 | A |
| 139 | A |
| 140 | A |
| 141 | A |
| 142 | — |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | — |
| 156 | — |
| 157 | A |
| 158 | — |
| 159 | — |
| 160 | A |
| 161 | A |
| 162 | — |
| 163 | — |
| 164 | — |
| 165 | — |
| 166 | — |
| 167 | — |
| 168 | — |
| 169 | — |
| 170 | — |
| 171 | E |
| 172 | C |
| 173 | D |
| 174 | D |
| 175 | E |
| 176 | D |
| 177 | — |
| 178 | — |
| 179 | — |
| 180 | B |
| 181 | A |
| 182 | A |
| 183 | A |
| 184 | A |
| 185 | B |
| 186 | A |
| 187 | B |
| 188 | — |
| 189 | — |
| 190 | — |
| 191 | — |
| 192 | — |
| 193 | — |
| 194 | — |
| 195 | — |
| 196 | — |
| 197 | — |
| 198 | — |
| 199 | — |
| 200 | — |

For Table XVI
A = ≦10 nM
B = 10.1 nM-25.0 nM
C = 25.1 nM-50.0 nM
D = 50.1 nM-100 nM
E = >100 nM

What is claimed is:

1. A compound of formula I 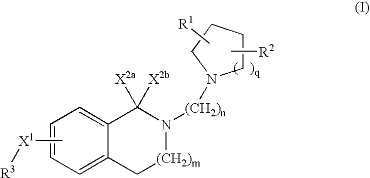
wherein
X¹ is (CR⁴R⁵), CO or O;
X^{2a} and X^{2b} are each H or are taken together to form ═O;
m is 0, 1 or 2;
n is 2, 3 or 4;
p is 0, 1 or 2;
q is 1, 2 or 3;
R¹ and R² are each independently H, halogen or an alkyl or haloalkyl group each group optionally substituted;
R³ is NR⁶R⁷ or an alkyl, cycloalkyl, cycloheteroalkyl, aryl or heteroaryl group each group optionally substituted with the proviso that when X¹ is O then R³ must be other than NR⁶R⁷; when X^{2a} and X^{2b} are taken together to form ═O and p is 0, then R³is not quinoxalinyl-2(1H)-one or an optionally substituted 1,3,4-oxadiazole; and when X^{2a} and X^{2b} are H and p is 0, then R³is not an optionally substituted 1,2,4-triazol-5(4H)-one;
R⁴ and R⁵ are each independently H or an optionally substituted alkyl or cycloalkyl group; and
R⁶ and R⁷ each independently H or an alkyl, alkenyl, alkoxy, cycloalkyl, cycloheteroalkyl, aryl or heteroaryl group each group optionally substituted or R⁶ and R⁷ may be taken together with the atom to which they are attached to form an optionally substituted 4- to 7-membered ring optionally containing one or two additional heteroatoms selected from N, O or S or an optionally substituted fused bicyclic or tricyclic 9- to 15-membered aromatic ring system optionally containing one to three additional heteroatoms selected from N, O or S; or
a stereoisomer, tautomer or pharmaceutically acceptable salt thereof.

2. The compound of claim 1 wherein X¹ is (CR⁴R⁵)ₚ or O; or a stereoisomer, tautomer or pharmaceutically acceptable salt thereof.

3. The compound of claim 1 wherein X¹ is (CR⁴R⁵)ₚ and p is 0; or a stereoisomer, tautomer or pharmaceutically acceptable salt thereof.

4. The compound of claim 1 wherein R¹ and R² are each independently H or methyl; or a stereoisomer, tautomer or pharmaceutically acceptable salt thereof.

5. The compound of claim 1 wherein X^{2a} and X^{2b} are each H; or a stereoisomer, tautomer or pharmaceutically acceptable salt thereof.

6. The compound of claim 1 wherein X^{2a} and X^{2b} are taken together to form ═O; or a stereoisomer, tautomer or pharmaceutically acceptable salt thereof.

7. The compound of claim 1 wherein R³ is an optionally substituted aminocarbonylphenyl or cycloheteroalkylcarbonylphenyl group; or a stereoisomer, tautomer or pharmaceutically acceptable salt thereof.

8. The compound of claim 1 wherein R³ is selected from the group consisting of phenyl, halophenyl, dihalophenyl, perhaloalkoxyphenyl, cyanophenyl, perhaloalkylphenyl, alkoxyphenyl, alkoxycarbonylphenyl, heteroaryl, cycloheteroalkylcarbonyl, cycloheteroalkylcarbonylphenyl, cyanoheteroaryl, carboxyphenyl, cycloalkylaminocarbonylphenyl, N,N-dialkylaminocarbonylphenyl, alkylaminocarbonylphenyl, alkycycloheteroalkylcarbonylphenyl, aminocarbonylphenyl, alkylaminocarbonylheteroaryl, cycloalkylcarbonylphenyl, cyanophenylalkoxy and dihydroisoquinolinone; or a stereoisomer, tautomer or pharmaceutically acceptable salt thereof.

9. The compound of claim 1 having the structure of formula Ia 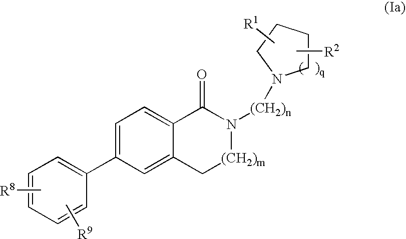
wherein
R⁸ and R⁹ are each independently H, halogen, CN, CONR¹⁰R¹¹, OR¹², CO₂R¹², COR¹², or an alkyl, haloalkyl or cycloalkyl group each group optionally substituted;
R¹⁰ and R¹¹ are each independently H or an alkyl, haloalkyl, cycloalkyl, aryl or heteroaryl group each group optionally substituted or R¹⁰ and R¹¹ may be taken together with the atom to which they are attached to form an optionally substituted 4- to 7-membered ring optionally containing one or two additional heteroatoms selected from N, O or S; and
R¹² is H or an alkyl, haloalkyl, cycloalkyl, alkenyl, alkynyl, aryl or heteroaryl group each group optionally substituted; or
a stereoisomer, tautomer or pharmaceutically acceptable salt thereof.

10. The compound of claim 9 wherein R⁸ is H or halogen, and R⁹ is CONR¹⁰R¹¹; or a stereoisomer, tautomer or pharmaceutically acceptable salt thereof.

11. The compound of claim 1 wherein m is 0 or 1; or a stereoisomer, tautomer or pharmaceutically acceptable salt thereof.

12. The compound of claim 1 wherein n is 2 or 3; or a stereoisomer, tautomer or pharmaceutically acceptable salt thereof.

13. The compound of claim 1 wherein q is 1 or 2; or a stereoisomer, tautomer or pharmaceutically acceptable salt thereof.

14. The compound of claim 1 selected from the group consisting of:
6-(4-fluorophenyl)-2-(2-pyrrolidin-1-ylethyl)-3,4-dihydroisoquinolin-1(2H)-one;
6-(3,5-difluorophenyl)-2-(2-pyrrolidin-1-ylethyl)-3,4-dihydroisoquinolin-1(2H)-one;
6-(2,4-difluorophenyl)-2-(2-pyrrolidin-1-ylethyl)-3,4-dihydroisoquinolin-1(2H)-one;
6-(2-fluorophenyl)-2-(2-pyrrolidin-1-ylethyl)-3,4-dihydroisoquinolin-1(2H)-one;
2-(2-pyrrolidin-1-ylethyl)-6-[3-(trifluoromethoxy)phenyl]-3,4-dihydroisoquinolin-1(2H)-one;
2-(2-pyrrolidin-1-ylethyl)-6-[4-(trifluoromethoxy)phenyl]-3,4-dihydroisoquinolin-1(2H)-one;
3-[1-oxo-2-(2-pyrrolidin-1-ylethyl)-1,2,3,4-tetrahydroisoquinolin-6-yl]benzonitrile;
6-phenyl-2-(2-pyrrolidin-1-ylethyl)-3,4-dihydroisoquinolin-1(2H)-one;
6-(3,4-difluorophenyl)-2-(2-pyrrolidin-1-ylethyl)-3,4-dihydroisoquinolin-1(2H)-one;
6-(3-fluorophenyl)-2-(2-pyrrolidin-1-ylethyl)-3,4-dihydroisoquinolin-1(2H)-one;
4-[1-oxo-2-(2-pyrrolidin-1-ylethyl)-1,2,3,4-tetrahydroisoquinolin-6-yl]benzonitrile;
2-(2-pyrrolidin-1-ylethyl)-6-[3-(trifluoromethyl)phenyl]-3,4-dihydroisoquinolin-1(2H)-one;
6-(1,3-benzodioxol-5-yl)-2-(2-pyrrolidin-1-ylethyl)-3,4-dihydroisoquinolin-1(2H)-one;
2-(2-pyrrolidin-1-ylethyl)-6-[4-(trifluoromethyl)phenyl]-3,4-dihydroisoquinolin-1(2H)-one;
6-(4-methoxyphenyl)-2-(2-pyrrolidin-1-ylethyl)-3,4-dihydroisoquinolin-1(2H)-one;
methyl 4-(1-oxo-2-(2-(pyrrolidin-1-yl)ethyl)-1,2,3,4-tetrahydroisoquinolin-6-yl)benzoate;
methyl 4-(2-{2-[(2R)-2-methylpyrrolidin-1-yl]ethyl}-1-oxo-1,2,3,4-tetrahydroiso-quinolin-6-yl)benzoate;
4-(2-{2-[(2R)-2-methylpyrrolidin-1-yl]ethyl}-1-oxo-1,2,3,4-tetrahydroisoquinolin-7-yl)benzonitrile;
3-(2-{2-[(2R)-2-methylpyrrolidin-1-yl]ethyl}-1-oxo-1,2,3,4-tetrahydroisoquinolin-5-yl)benzonitrile;
4-(2-{2-[(2R)-2-methylpyrrolidin-1-yl]ethyl}-1-oxo-1,2,3,4-tetrahydroisoquinolin-5-yl)benzonitrile;
4-[1-oxo-2-(3-pyrrolidin-1-ylpropyl)-1,2,3,4-tetrahydroisoquinolin-6-yl]benzonitrile;
3-[1-oxo-2-(3-pyrrolidin-1-ylpropyl)-1,2,3,4-tetrahydroisoquinolin-6-yl]benzonitrile;
6-pyridin-4-yl-2-(2-pyrrolidin-1-ylethyl)-3,4-dihydroisoquinolin-1(2H)-one;
1-[1-oxo-2-(2-pyrrolidin-1-ylethyl)-1,2,3,4-tetrahydroisoquinolin-6-yl]-1H-indole-5-carbonitrile;
6-(pyrrolidin-1-ylcarbonyl)-2-(2-pyrrolidin-1-ylethyl)-3,4-dihydroisoquinolin-1(2H)-one;
6-(4-fluorophenyl)-2-{2-[(2S)-2-methylpyrrolidin-1-yl]ethyl}-3,4-dihydroisoquinolin-1(2H)-one;
6-(4-fluorophenyl)-2-{2-[(2R)-2-methylpyrrolidin-1-yl]ethyl}-3,4-dihydroisoquinolin-1(2H)-one;
4-(2-{2-[(2S)-2-methylpyrrolidin-1-yl]ethyl}-1-oxo-1,2,3,4-tetrahydroisoquinolin-6-yl)benzonitrile;
4-(2-{2-[(2R)-2-methylpyrrolidin-1-yl]ethyl}-1-oxo-1,2,3,4-tetrahydroisoquinolin-6-yl)benzonitrile;
6-{[1-oxo-2-(2-pyrrolidin-1-ylethyl)-1,2,3,4-tetrahydroisoquinolin-6-yl]oxy}nicotinonitrile;
6-[(2-{2-[(2R)-2-methylpyrrolidin-1-yl]ethyl}-1-oxo-1,2,3,4-tetrahydroisoquinolin-6-yl)oxy]nicotinonitrile;
4-[(2-{2-[(2R)-2-methylpyrrolidin-1-yl]ethyl}-1-oxo-1,2,3,4-tetrahydroisoquinolin-6-yl)oxy]benzonitrile;
4-{[1-oxo-2-(2-pyrrolidin-1-ylethyl)-1,2,3,4-tetrahydroisoquinolin-6-yl]oxy}benzonitrile;
5-[(2-{2-[(2R)-2-methylpyrrolidin-1-yl]ethyl}-1-oxo-1,2,3,4-tetrahydroisoquinolin-6yl)oxy]pyridine-2-carbonitrile;
5-{[1-oxo-2-(2-pyrrolidin-1-ylethyl)-1,2,3,4-tetrahydroisoquinolin-6-yl]oxy}pyridine-2-carbonitrile;
6-[4-(pyrrolidin-1-ylcarbonyl)phenyl]-2-(2-pyrrolidin-1-ylethyl)-3,4-dihydroisoquinolin-1(2H)-one;
N-cyclopentyl-4-[1-oxo-2-(2-pyrrolidin-1-ylethyl)-1,2,3,4-tetrahydroisoquinolin-6-yl]benzamide;
N,N-dimethyl-4-[1-oxo-2-(2-pyrrolidin-1-ylethyl)-1,2,3,4-tetrahydroisoquinolin-6-yl]benzamide;
N-cyclopropyl-4-[1-oxo-2-(2-pyrrolidin-1-ylethyl)-1,2,3,4-tetrahydroisoquinolin-6-yl]benzamide;
N-ethyl-4-[1-oxo-2-(2-pyrrolidin-1-ylethyl)-1,2,3,4-tetrahydroisoquinolin-6-yl]benzamide;
N-methyl-4-[1-oxo-2-(2-pyrrolidin-1-ylethyl)-1,2,3,4-tetrahydroisoquinolin-6-yl]benzamide;
N-(cyclopropylmethyl)-4-[1-oxo-2-(2-pyrrolidin-1-ylethyl)-1,2,3,4-tetrahydroiso-quinolin-6-yl]benzamide;
N-isopropyl-4-[1-oxo-2-(2-pyrrolidin-1-ylethyl)-1,2,3,4-tetrahydroisoquinolin-6-yl]benzamide;
N,N-diethyl-4-[1-oxo-2-(2-pyrrolidin-1-ylethyl)-1,2,3,4-tetrahydroisoquinolin-6yl]benzamide;
N-cyclobutyl-4-[1-oxo-2-(2-pyrrolidin-1-ylethyl)-1,2,3,4-tetrahydroisoquinolin-6-yl]benzamide;
6-[4-(azetidin-1-ylcarbonyl)phenyl]-2-(2-pyrrolidin-1-ylethyl)-3,4-dihydroisoquinolin-1(2H)-one;
N,N-diethyl-4-(2-{2-[(2R)-2-methylpyrrolidin-1-yl]ethyl}-1-oxo-1,2,3,4-tetrahydroisoquinolin-6-yl)benzamide;
2-{2-[(2R)-2-methylpyrrolidin-1-yl]ethyl}-6-[4-(pyrrolidin-1-ylcarbonyl)phenyl]-3,4-dihydroisoquinolin-1(2H)-one;
4-[1-oxo-2-(2-pyrrolidin-1-ylethyl)-1,2,3,4-tetrahydroisoquinolin-6-yl]benzamide;
N-(2-fluoroethyl)-4-[1-oxo-2-(2-pyrrolidin-1-ylethyl)-1,2,3,4-tetrahydroisoquinolin-6-yl]benzamide;
N-(2-methoxyethyl)-4-[1-oxo-2-(2-pyrrolidin-1-ylethyl)-1,2,3,4-tetrahydroisoquinolin-6-yl]benzamide;
N-(2-isopropoxyethyl)-4-[1-oxo-2-(2-pyrrolidin-1-ylethyl)-1,2,3,4-tetrahydroisoquinolin-6-yl]benzamide;
4-[1-oxo-2-(2-pyrrolidin-1-ylethyl)-1,2,3,4-tetrahydroisoquinolin-6-yl]-N-(2-phenoxyethyl)benzamide;
N-(2-ethoxyethyl)-4-[1-oxo-2-(2-pyrrolidin-1-ylethyl)-1,2,3,4-tetrahydroisoquinolin-6-yl]benzamide;
N-(cyclopropylmethyl)-4-(2-{2-[(2R)-2-methylpyrrolidin-1-yl]ethyl}-1-oxo-1,2,3,4-tetrahydroisoquinolin-6-yl)benzamide;
N-cyclobutyl-4-(2-{2-[(2R)-2-methylpyrrolidin-1-yl]ethyl}-1-oxo-1,2,3,4-tetrahydroisoquinolin-6-yl)benzamide;
N-ethyl-4-(2-{2-[(2R)-2-methylpyrrolidin-1-yl]ethyl}-1-oxo-1,2,3,4-tetrahydroisoquinolin-6-yl)benzamide;
N-cyclopropyl-4-(2-{2-[(2R)-2-methylpyrrolidin-1-yl]ethyl}-1-oxo-1,2,3,4-tetrahydroisoquinolin-6-yl)benzamide;
N-isopropyl-4-(2-{2-[(2R)-2-methylpyrrolidin-1-yl]ethyl}-1-oxo-1,2,3,4-tetrahydroisoquinolin-6-yl)benzamide;
N-methyl-4-(2-{2-[(2R)-2-methylpyrrolidin-1-yl]ethyl}-1-oxo-1,2,3,4-tetrahydroisoquinolin-6-yl)benzamide;
6-[4-(piperidin-1-ylcarbonyl)phenyl]-2-(2-pyrrolidin-1-ylethyl)-3,4-dihydroisoquinolin-1(2H)-one;
N-cyclopentyl-4-(2-{2-[(2R)-2-methylpyrrolidin-1-yl]ethyl}-1-oxo-1,2,3,4-tetrahydroisoquinolin-6-yl)benzamide;
6-(4-{[(2S)-2-methylpyrrolidin-1-yl]carbonyl}phenyl)-2-(2-pyrrolidin-1-ylethyl)-3,4,-dihydroisoquinolin-1(2H)-one;
6-(4-{[(2R)-2-methylpyrrolidin-1-yl]carbonyl}phenyl)-2-(2-pyrrolidin-1-ylethyl)-3,4-dihydroisoquinolin-1(2H)-one;
N-methyl-4-(2-{2-[(2R)-2-methylpyrrolidin-1-yl]ethyl}-1-oxo-1,2,3,4-tetrahdyroiso-quinolin-7-yl)benzamide;
N-ethyl-4-(2-{2-[(2R)-2-methylpyrrolidin-1-yl]ethyl}-1-oxo-1,2,3,4-tetrahydroiso-quinolin-7-yl)benzamide;
N-isopropyl-4-(2-{2-[(2R)-2-methylpyrrolidin-1-yl]ethyl}-1-oxo-1,2,3,4-tetrahydroiso-quinolin-7-yl)benzamide;
2-{2-[(2R)-2-methylpyrrolidin-1-yl]ethyl}-7-[4-(pyrrolidin-1-ylcarbonyl)phenyl]-3,4-dihydroisoquinolin-1(2H)-one;
4-{[1-oxo-2-(2-pyrrolidin-1-ylethyl)-1,2,3,4-tetrahydroisoquinolin-6-yl]oxy}benzamide
N-methyl-4-{[1-oxo-2-(2-pyrrolidin-1-ylethyl)-1,2,3,4-tetrahydroisoquinolin-6-yl]oxy}benzamide
N-ethyl-4-{[1-oxo-2-(2-pyrrolidin-1-ylethyl)-1,2,3,4-tetrahydroisoquinolin-6-yl]oxy}benzamide;
N-isopropyl-4-{[1-oxo-2-(2-pyrrolidin-1-ylethyl)-1,2,3,4-tetrahydroisoquinolin-6-yl]oxy}benzamide;
N,N-dimethyl-4-{[1-oxo-2-(2-pyrrolidin-1-ylethyl)-1,2,3,4-tetrahydroisoquinolin-6-yl]oxy}benzamide;
N,N-diethyl-4-{[1-oxo-2-(2-pyrrolidin-1-ylethyl)-1,2,3,4-tetrahydroisoquinolin-6-yl]oxy}benzamide;
N-cyclobutyl-4-{[1-oxo-2-(2-pyrrolidin-1-ylethyl)-1,2,3,4-tetrahydroisoquinolin-6-yl]oxy}benzamide;
6-[4-(pyrrolidin-1-ylcarbonyl)phenoxy]-2-(2-pyrrolidin-1-ylethyl)-3,4-dihydroiso-quinolin-1(2H)-one;
N-cyclopropyl-4-{[1-oxo-2-(2-pyrrolidin-1-ylethyl)-1,2,3,4-tetrahydroisoquinolin-6-yl]oxy}benzamide;
N-methyl-6-{[1-oxo-2-(2-pyrrolidin-1-ylethyl)-1,2,3,4-tetrahydroisoquinolin-6-yl]oxy}nicotinamide;
N-methoxy-N-methyl-4-(1-oxo-2-(2-(pyrrolidin-1-yl)ethyl)-1,2,3,4-tetrahydroisoquinolin-6-yl)benzamide;
6-[4-(cyclopropylcarbonyl)phenyl]-2-(2-pyrrolidin-1-ylethyl)-3,4-dihydroisoquinolin-1(2H)-one;
6-(1H-benzimidazol-1-yl)-2-(2-pyrrolidin-1-ylethyl)-3,4-dihydroisoquinolin-1(2H)-one;
5-(1H-benzimidazol-1-ylmethyl)-2-(2-pyrrolidin-1-ylethyl)isoindolin-1-one;
6-(4-fluorophenyl)-2-(2-piperidin-1-ylethyl)-3,4-dihydroiso-quinolin-1(2H-one;
4-[1-oxo-2-(3-piperidin-1-ylpropyl)-1,2,3,4-tetrahydroisoquinolin-6-yl]benzonitrile;
2-(2-azepan-1-ylethyl)-6-(4-fluorophenyl)-3,4-dihydroisoquinolin-1(2H)-one;
4-{[1-oxo-2-(2-pyrrolidin-1-ylethyl)-1,2,3,4-tetrahydroisoquinolin-6-yl]oxy}benzamide;
4-[1-oxo-2-(2-pyrrolidin-1-ylethyl)-1,2,3,4-tetrahydroisoquinolin-6-yl]benzoic acid;
4-{[1-oxo-2-(2-pyrrolidin-1-ylethyl)-1,2,3,4-tetrahydroisoquinolin-6-yl]oxy}benzoic acid;
(R)-4-(2-(2-(2-methylpyrrolidin-1-yl)ethyl)-1-oxoisoindolin-5-yl)benzonitrile;
4-{[(2-{2-[(2R)-2-methylpyrrolidin-1-yl]ethyl}-1-oxo-1,2,3,4-tetrahydroisoquinolin-6-yl)oxy]methyl}benzonitrile;
4-[1-oxo-2-(2-pyrrolidin-1-ylethyl)-1,2,3,4-tetrahydroisoquinolin-6-yl]-N-(2-thienylmethyl)benzamide;
6-[4-(morpholin-4-ylcarbonyl)phenyl]-2-(2-pyrrolidin-1-ylethyl)-3,4-dihydroisoquinolin-1(2H)-one;
N-(2-chloroethyl)-4-[1-oxo-2-(2-pyrrolidin-1-ylethyl)-1,2,3,4-tetrahydroisoquinolin-6-yl]benzamide;
N-ethyl-N-methyl-4-[1-oxo-2-(2-pyrrolidin-1-ylethyl)-1,2,3,4-tetrahydroisoquinolin-6-yl]benzamide;
N-(2-furylmethyl)-4-[1-oxo-2-(2-pyrrolidin-1-ylethyl)-1,2,3,4-tetrahydroisoquinolin-6-yl]benzamide;
N-[(1 S)-2-methoxy-1-methylethyl]-4-[1-oxo-2-(2-pyrrolidin-1-ylethyl)-1,2,3,4-tetrahydroisoquinolin-6-yl]benzamide;
6-{4-[(3-methoxypyrrolidin-1-yl)carbonyl]phenyl}-2-(2-pyrrolidin-1-ylethyl)-3,4-dihydroisoquinolin-1(2H)-one;
6-(4-{[(2S)-2-(methoxymethyl)pyrrolidin-1-yl]carbonyl}phenyl)-2-(2-pyrrolidin-1-ylethyl)-3,4-dihydroisoquinolin-1(2H)-one;
4-[1-oxo-2-(2-pyrrolidin-1-ylethyl)-1,2,3,4-tetrahydroisoquinolin-6-yl]-N-propylbenzamide;
4-[1-oxo-2-(2-pyrrolidin-1-ylethyl)-1,2,3,4-tetrahydroisoquinolin-6-yl]-N-1,3-thiazol-2-ylbenzamide;
6-[4-fluoro-3-(pyrrolidin-1-ylcarbonyl)phenyl]-2-(2-pyrrolidin-1-ylethyl)-3,4-dihydroisoquinolin-1(2H)-one;
2-fluoro-N,N-dimethyl-5-[1-oxo-2-(2-pyrrolidin-1-ylethyl)-1,2,3,4-tetrahydroisoquinolin-6-yl]benzamide;
3-fluoro-N,N-dimethyl-4-[1-oxo-2-(2-pyrrolidin-1-ylethyl)-1,2,3,4-tetrahydroisoquinolin-6-yl]benzamide;
6-[2-fluoro-4-(pyrrolidin-1-ylcarbonyl)phenyl]-2-(2-pyrrolidin-1-ylethyl)-3,4-dihydroisoquinolin-1(2H)-one;
6-[3-fluoro-4-(pyrrolidin-1-ylcarbonyl)phenyl]-2-(2-pyrrolidin-1-ylethyl)-3,4-dihydroisoquinolin-1(2H)-one;
6-[3-chloro-4-(pyrrolidin-1-ylcarbonyl)phenyl]-2-(2-pyrrolidin-1-ylethyl)-3,4-dihydroisoquinolin-1(2H)-one;
2-{2-[(2R)-2-methylpyrrolidin-1-yl]ethyl}-6-[4-(pyrrolidin-1-ylcarbonyl)phenoxy]-3,4-dihydroisoquinolin-1(2H)-one;
N-ethyl-4-[2-(2-pyrrolidin-1-ylethyl)-1,2,3,4-tetrahydroisoquinolin-6-yl]benzamide;
N-methyl-4-[2-(2-pyrrolidin-1-ylethyl)-1,2,3,4-tetrahydroisoquinolin-6-yl]benzamide;
6-[4-(pyrrolidin-1-ylcarbonyl)phenyl]-2-(2-pyrrolidin-1-ylethyl)-1,2,3,4-tetrahydroisoquinoline;
N,N-dimethyl-4-[2-(2-pyrrolidin-1-ylethyl)-1,2,3,4-tetrahydroisoquinolin-6-yl]benzamide;
6-[4-(piperidin-1-ylcarbonyl)phenyl]-2-(2-pyrrolidin-1-ylethyl)-1,2,3,4-tetrahydroisoquinoline;
6-[4-(morpholin-4-ylcarbonyl)phenyl]-2-(2-pyrrolidin-1-ylethyl)-1,2,3,4-tetrahydroisoquinoline;
4-[2-(2-pyrrolidin-1-ylethyl)-1,2,3,4-tetrahydroisoquinolin-6-yl]benzamide;
N-methyl-4-[1-oxo-2-(3-pyrrolidin-1-ylpropyl)-1,2,3,4-tetrahydroisoquinolin-6-yl]benzamide;
6-(4-{[(2S)-2-methylpyrrolidin-1-yl]carbonyl}phenyl)-2-(2-pyrrolidin-1-ylethyl)-1,2,3,4-tetrahydroisoquinoline;
6-(1H-pyrazol-1-yl)-2-(2-pyrrolidin-1-ylethyl)-3,4-dihydroisoquinolin-1(2H)-one;
6-(1H-indazol-1-yl)-2-(2-pyrrolidin-1-ylethyl)-3,4-dihydroisoquinolin-1(2H)-one;
2-{2-[(2R)-2-methylpyrrolidin-1-yl]ethyl}-3,3′,4,4′-tetrahydro-6,6′-biisoquinoline-1,1′(2H,2′H)-dione;
6-(azepan-1-ylcarbonyl)-2-{2-[(2R)-2-methylpyrrolidin-1-yl]ethyl}-3,4-dihydroiso-quinolin-1(2H)-one;
N-cyclobutyl-2-{2-[(2R)-2-methylpyrrolidin-1-yl]ethyl}-1-oxo-1,2,3,4-tetrahydroiso-quinoline-6-carboxamide;
2-{2-[(2R)-2-methylpyrrolidin-1-yl]ethyl}-6-(piperidin-1-ylcarbonyl)-3,4-dihydro-isoquinolin-1(2H)-one;
N-cyclohexyl-2-{2-[(2R)-2-methylpyrrolidin-1-yl]ethyl}-1-oxo-1,2,3,4-tetrahydroiso-quinoline-6-carboxamide;
N-(2,3-dihydro-1H-inden-2-yl)-2-{2-[(2R)-2-methylpyrrolidin-1-yl]ethyl}-1-oxo-1,2,3,4-tetrahydroisoquinoline-6-carboxamide;
2-{2-[(2R)-2-methylpyrrolidin-1-yl]ethyl}-1-oxo-N-pyridin-4-yl-1,2,3,4-tetrahydroisoqui-noline-6-carboxamide;
N-cyclopentyl-2-{2-[(2R)-2-methylpyrrolidin-1-yl]ethyl}-1-oxo-1,2,3,4-tetrahydroiso-quinoline-6-carboxamide;
6-(3,4-dihydroisoquinolin-2(1H)-ylcarbonyl)-2-{2-[(2R)-2-methylpyrrolidin-1-yl]ethyl}-3,4-dihydroisoquinolin-1(2H)-one;
2-{2-[(2R)-2-methylpyrrolidin-1-yl]ethyl}-6-(pyrrolidin-1-ylcarbonyl)-3,4-dihydroiso-quinolin-1(2H)-one;
6-(1,3-dihydro-2H-isoindol-2-ylcarbonyl)-2-{2-[(2R)-2-methylpyrrolidin-1-yl]ethyl}-dihydroisoquinolin-1(2H)-one;
6-(4-fluorophenyl)-2-{2-[(2R)-2-methylpyrrolidin-1-yl]ethyl}-1,2,3,4-tetrahydroiso-quinoline;
2-(2-pyrrolidin-1-ylethyl)-6-[4-(trifluoromethoxy)phenyl]-1,2,3,4-tetrahydroisoquinoline;
6-(3-fluorophenyl)-2-(2-pyrrolidin-1-ylethyl)-1,2,3,4-tetrahydroisoquinoline;
6-(1,3-benzodioxol-5-yl)-2-(2-pyrrolidin-1-ylethyl)-1,2,3,4-tetrahydroisoquinoline;
6-(4-fluorophenyl)-2-(2-piperidin-1-ylethyl)-1,2,3,4-tetrahydroisoquinoline;
2-(2-azepan-1-ylethyl)-6-(4-fluorophenyl)-1,2,3,4-tetrahydroisoquinoline;
3-fluoro-N-methyl-4-[1-oxo-2-(2-pyrrolidin-1-ylethyl)-1,2,3,4-tetrahydroisoquinolin-6-yl]benzamide;
N-ethyl-3-fluoro-4-[1-oxo-2-(2-pyrrolidin-1-ylethyl)-1,2,3,4-tetrahydroisoquinolin-6-yl]benzamide;
6-(1H-benzimidazol-1-yl)-2-{2-[(2R)-2-methylpyrrolidin-1-yl]ethyl}-3,4-dihydroiso-quinolin-1(2H)-one;
2-{2-[(2R)-2-methylpyrrolidin-1-yl]ethyl}-5-[4-(pyrrolidin-1-ylcarbonyl)phenyl]-isoindolin-1-one;
N-methyl-4-(2-{2-[(2R)-2-methylpyrrolidin-1-yl]ethyl}-1-oxo-2,3-dihydro-1H-isoindol-5-yl)benzamide;
6-[4-(methylsulfonyl)phenyl]-2-(2-pyrrolidin-1-ylethyl)-3,4-dihydroisoquinolin-1(2H)-one;
2-{2-[(2R)-2-methylpyrrolidin-1-yl]ethyl}-6-piperidin-1-yl-3,4-dihydroisoquinolin-1(2H)-one;
6-(piperidin-1-yl)-2-(2-(pyrrolidin-1-yl)ethyl)-3,4-dihydroisoquinolin-1(2H)-one;
6-(piperidin-1-yl)-2-(2-(piperidin-1-yl)ethyl)-3,4-dihydroisoquinolin-1(2H)-one;
2-(2-(azepan-1-yl)ethyl)-6-(piperidin-1-yl)-3,4-dihydroisoquinolin-1(2H)-one;
(R)-2-(2-(2-methylpyrrolidin-1-yl)ethyl)-6-(pyrrolidin-1-yl)-3,4-dihydroisoquinolin-1(2H)-one;
(R)-6-(azepan-1-yl)-2-(2-(2-methylpyrrolidin-1-yl)ethyl)-3,4-dihydroisoquinolin-1(2H)-one;
(R)-2-methyl-2′-(2-(2-methylpyrrolidin-1-yl)ethyl)-3,3′,4,4′-tetrahydro-6,6′-biisoqui-noline-1,1′(2H,2′H)-dione;
2-methyl-2′-(2-(pyrrolidin-1-yl)ethyl)-3,3′,4,4′-tetrahydro-6,6′-biisoquinoline-1,1′(2H,2′H)-dione;
2-(3-(pyrrolidin-1-yl)propyl)-6-(4-(pyrrolidine-1-carbonyl)phenyl)-3,4-dihydroiso-quinolin-1(2H)-one;
6-(isoindoline-2-carbonyl)-2-(2-(pyrrolidin-1-yl)ethyl)-3,4-dihydroisoquinolin-1(2H)-one;
6-(piperidine-1-carbonyl)-2-(2-(pyrrolidin-1-yl)ethyl)-3,4-dihydroisoquinolin-1(2H)-one;
(R)-N,N-dimethyl-4-(2-(2-(2-methylpyrrolidin-1-yl)ethyl)-1-oxo-1,2,3,4-tetrahydroiso-quinolin-6-yl)benzamide;
(R)-6-(4-(azetidine-1-carbonyl)phenyl)-2-(2-(2-methylpyrrolidin-1-yl)ethyl)-3,4-dihydroisoquinolin-1(2H)-one;
(R)-2-(2-(2-methylpyrrolidin-1-yl)ethyl)-6-(4-(piperidine-1-carbonyl)phenyl)-3,4-dihydroisoquinolin-1(2H)-one;
(R)-2-(2-(2-methylpyrrolidin-1-yl)ethyl)-6-(4-(morpholine-4-carbonyl)phenyl)-3,4-dihydroisoquinolin-1(2H)-one;
(R)-N-(2-methoxyethyl)-4-(2-(2-(2-methylpyrrolidin-1-yl)ethyl)-1-oxo-1,2,3,4-tetrahydroisoquinolin-6-yl)benzamide;
(R)-N-(2-isopropoxyethyl)-4-(2-(2-(2-methylpyrrolidin-1-yl)ethyl)-1-oxo-1,2,3,4-tetrahydroisoquinolin-6-yl)benzamide;
N-((S)-1-methoxypropan-2-yl)-4-(2-(2-((R)-2-methylpyrrolidin-1-yl)ethyl)-1-oxo-1,2,3,4-tetrahydroisoquinolin-6-yl)benzamide;
(R)-N-(2-fluoroethyl)-4-(2-(2-(2-methylpyrrolidin-1-yl)ethyl)-1-oxo-1,2,3,4-tetrahydroisoquinolin-6-yl)benzamide;
6-(4-((S)-2-(methoxymethyl)pyrrolidine-1-carbonyl)phenyl)-2-(2-((R)-2-methylpyrrolidin-1-yl)ethyl)-3,4-dihydroisoquinolin-1(2H)-one; and
(R)-N-ethyl-N-methyl-4-(2-(2-(2-methylpyrrolidin-1-yl)ethyl)-1-oxo-1,2,3,4-tetrahydroisoquinolin-6-yl)benzamide; or
a tautomer or pharmaceutically acceptable salt thereof.

15. A method for the treatment of a patient suffering from a cognitive disorder related to or affected by the Histamine-3 (H₃) receptor comprising admistering to the patient a compound of formula I 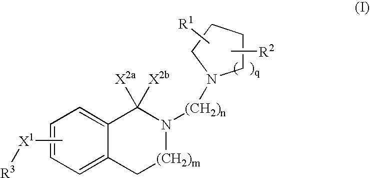
wherein
X¹ is (CR⁴R⁵)ₚ, CO or O;
X^{2a} and X^{2b} are each H or are taken together to form ═O;
m is 0, 1 or 2;
n is 2,3 or 4;
p is 0, 1 or 2;
q is 1, 2 or 3;
R¹ and R² are each independently H, halogen or an alkyl or haloalkyl group each group optionally substituted;
R³ is NR⁶R⁷ or an alkyl, cycloalkyl, cycloheteroalkyl, aryl or heteroaryl group each group optionally substituted with the proviso that when X¹ is O then R³ must be other than NR⁶R⁷;
R⁴ and R⁵ are each independently H or an optionally substituted alkyl or cycloalkyl group; and
R⁶ and R⁷ each independently H or an alkyl, alkenyl, alkoxy, cycloalkyl, cycloheteroalkyl, aryl or heteroaryl group each group optionally substituted or R⁶ and R⁷ may be taken together with the atom to which they are attached to form an optionally substituted 4- to 7-membered ring optionally containing one or two additional heteroatoms selected from N, O or S or an optionally substituted fused bicyclic or tricyclic 9- to 15-membered aromatic ring system optionally containing one to three additional heteroatoms selected from N, O or S; or
a stereoisomer, tautomer or pharmaceutically acceptable salt thereof.

16. The method of claim 15, wherein said disorder is a neurodegenerative disorder.

17. The method of claim 15, wherein said disorder is mild cognitive impairment (MCl), dementia, delirium, amnestic disorder, Alzheimer's disease (AD), Parkinson's disease (PD), Huntington's disease (HD), memory disorder, memory deficits associated with depression, schizophrenia, a psychotic disorder, paranoia, mano-depressive illness, attention deficit hyperactivity disorder (ADHD), dyslexia, developmental disorders, Down's syndrome, Fragile X syndrome, loss of executive function, loss of learned information, vascular dementia, cognitive decline, neurodegenerative disorder, HIV-induced dimentia, head trauma, Pick's disease, Creutzfeldt-Jakob disease, Body dementia, vascular dementia, surgical procedure-induced cognitive dysfunction, traumatic brain injury or stroke.

18. The method of claim 15, wherein said disorder is selected from the group consisting of: Alzheimer's disease, attention deficit disorder, schizophrenia, cognitive dysfunction in schizophrenia, Parkinsons' disease, frontal temporal dementia or depression.

19. A pharmaceutical composition which comprises a pharmaceutically acceptable carrier and an effective amount of a compound according to claim 1.

20. A process for the preparation of a compound of formula I 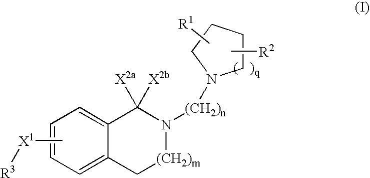
wherein
X¹ is (CR⁴R⁵)ₚ, CO or O;
X^{2a} and X^{2b} are each H or are taken together to form ═O;
m is 0, 1 or 2;
n is 2, 3 or 4;
p is 0, 1 or 2;
q is 1, 2 or 3;
R¹ and R² are each independently H, halogen or an alkyl or haloalkyl group each group optionally substituted;
R³ is NR⁶R⁷ or an alkyl, cycloalkyl, cycloheteroalkyl, aryl or heteroaryl group each group optionally substituted with the proviso that when X¹ is O then R³ must be other than NR⁶R⁷;
R⁴ and R⁵ are each independently H or an optionally substituted alkyl or cycloalkyl group; and
R⁶ and R⁷ each independently H or an alkyl, alkenyl, alkoxy, cycloalkyl, cycloheteroalkyl, aryl or heteroaryl group each group optionally substituted or R⁶ and R⁷ may be taken together with the atom to which they are attached to form an optionally substituted 4- to 7-membered ring optionally containing one or two additional heteroatoms selected from N, O or S or an optionally substituted fused bicyclic or tricyclic 9- to 15-membered aromatic ring system optionally containing one to three additional heteroatoms selected from N, O or S; or
a stereoisomer, tautomer or pharmaceutically acceptable salt thereof
which process comprises reacting a compound of formula II 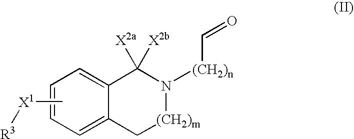
wherein X¹, X², R³, m and n are as described hereinabove for formula I with a pyrrolidine of formula III 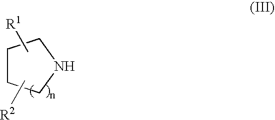
wherein R¹ and R² are as described hereinabove for formula I in the presence of a NaBH₃CN optionally in the presence of an acid optionally in the presence of a solvent.
